(19) United States
(12) Patent Application Publication
(10) Pub. No.: US 2025/0223344 A1
(43) Pub. Date: Jul. 10, 2025

(54) Compositions and Methods for Treatment of Liver Disease

(71) Applicant: The General Hospital Corporation, Boston, MA (US)

(72) Inventor: Michael Yoonsuk Choi, Boston, MA (US)

(21) Appl. No.: 18/824,691

(22) Filed: Sep. 4, 2024

Related U.S. Application Data
(63) Continuation of application No. 17/286,011, filed on Apr. 16, 2021, now abandoned.
(60) Provisional application No. 62/747,903, filed on Oct. 19, 2018.

(51) Int. Cl. C07K 16/22; A61K 31/7088; A61K 35/407; A61K 48/00; A61P 1/16; C12N 5/071; C12N 15/113

(52) U.S. Cl. CPC C07K 16/22; A61K 31/7088; A61K 35/407; A61P 1/16; C12N 5/067; C12N 15/1136; A61K 48/00; C12N 2310/141

(57) ABSTRACT

Described herein are methods and compositions for treating a liver disease. Aspects of the invention relate to administering to a subject an agent that inhibits WISP1. Another aspect of the invention relates to administering to a subject a HSC that expresses and agent that inhibits WISP1.

### CLAIM OF PRIORITY

This application is a continuation of U.S. patent application Ser. No. 17/286,011, filed Apr. 16, 2021, which is the U.S. National Phase Application under 35 U.S.C. § 371 of International Patent Application No. PCT/US2019/056910, filed on Oct. 18, 2019, which claims benefit under 35 U.S.C. § 119(e) U.S. Provisional Application No. 62/747,903, filed Oct. 19, 2018, the contents of which are incorporated herein by reference in its entirety.

### SEQUENCE LISTING

This application contains a Sequence Listing that has been submitted electronically as an XML file named “40978-0033002_SL_ST26.XML.” The XML file, created on Sep. 4, 2024, is 160,305 bytes in size. The material in the XML file is hereby incorporated by reference in its entirety.

### FIELD OF INVENTION

The field of the invention relates to a treatment for liver disease.

### BACKGROUND

The liver is a highly important organ that filters metabolites, synthesizes proteins, and can produce biochemicals needed for digestion. Specifically, the liver produces bile to breakdown fats and emulsify lipids. Chronic and progressive liver diseases can result in the progressive destruction of the bile ducts in the liver that can lead to a buildup of bile, severe inflammation, scarring, and fibrosis. When scar tissue replaces healthy liver tissue, the liver function becomes increasingly impaired. For some liver diseases, (e.g. primary biliary cholangitis (PBC)), there is only one drug, ursodeoxycholic acid (UDCA), that can improve survival. Unfortunately, approximately 40% of UDCA-treated patients show an inadequate response to the therapy. Therefore, more effective therapeutics are needed for the treatment of liver diseases such as PBC and others.

### SUMMARY OF THE INVENTION

The invention described herein relates to, in part, the finding that inhibition of WISP1 by miRNA-15a, miRNA-412, and anti-WISP1 antibodies induced quiescence in an activated hepatic stellate cells (HSCs), the cell type that plays a central role in fibrotic progression of the liver. It is further shown herein that miR-15a and WISP1 IgG can directly target WISP1 to repress the protein's pro-fibrotic function in activated HSCs.

Accordingly, one aspect described herein is a method for treating or preventing a liver disease, comprising administering to a subject in need thereof an antibody or antibody reagent that inhibits WISP1.

In one embodiment of any aspect, the liver disease is selected from the group consisting of: Alagille Syndrome; Alcohol-Related Liver Disease; Alpha-1 Antitrypsin Deficiency; Autoimmune Hepatitis; Benign Liver Tumors; Biliary Atresia; Cirrhosis; Crigler-Najjar Syndrome; Galactosemia; Gilbert Syndrome; Hemochromatosis; Hepatic Encephalopathy; Hepatitis A; Hepatitis B; Hepatitis C; Hepatorenal Syndrome; Intrahepatic Cholestasis of Pregnancy (ICP); Lysosomal Acid Lipase Deficiency (LAL-D); Liver Cysts; Liver Cancer; Newborn Jaundice; Non-Alcoholic Fatty Liver Disease; Non-Alcoholic Steatohepatitis; Primary Biliary Cholangitis (PBC); Primary Sclerosing Cholangitis (PSC); Progressive Familial Intrahepatic Cholestasis (PFIC); Reye Syndrome; Type I Glycogen Storage Disease; scleroderma; and Wilson Disease.

In one embodiment of any aspect, the WISP1 is a splice variant selected from the group consisting of: WISP1v, WISP1vx, and WISP1delta exon 3-4.

In one embodiment of any aspect, the antibody or antibody reagent that inhibits WISP1 is selected from the group consisting of: mab1680, AF1680, SAB2501114, ab60114, and ab65943.

In one embodiment of any aspect, the antibody or antibody reagent amino acid sequence comprises at least 70% homology to any one of SEQ ID NOs: 1-4, 6, or 12-120.

In one embodiment of any aspect, the subject is a mammal.

In one embodiment of any aspect, WISP1 is inhibited in a target cell. In one embodiment of any aspect, the target cell is a mammalian cell. In one embodiment of any aspect, the target cell is a hepatic stellate cell, a fibroblast, or a myofibroblast. In one embodiment of any aspect, the hepatic stellate cell is quiescent.

In one embodiment of any aspect, the antibody or antibody reagent is administered by direct injection, subcutaneous injection, muscular injection, oral, transdermal or nasal administration.

In one embodiment of any aspect, inhibiting WISP1 is inhibiting WISP1 activity or reducing WISP1 protein levels. In one embodiment of any aspect, the activity of WISP1 is inhibited by at least 50%, at least 60%, at least 70%, at least 80%, at least 90%, or more as compared to an appropriate control. In one embodiment of any aspect, the level of WISP1 is reduced by at least 50%, at least 60%, at least 70%, at least 80%, at least 90%, or more as compared to an appropriate control.

Another aspect described herein provides a composition comprising an antibody or antibody reagent that inhibits WISP1 and a pharmaceutically acceptable carrier.

In one embodiment of any aspect, the composition is formulated for treating or preventing a liver disease.

Another aspect described herein provides a method of treating a liver disease in a subject, the method comprising: (a) detecting the level of WISP1 and/or Yap, Colla1, Acta2 in a biological sample of a subject; (b) comparing the measurement of (a) to a reference level; (c) identifying a subject with increased WISP1 and/or Yap, Colla1, Acta2 in (a) as compared to a reference level as having a liver disease; and (d) administering to the subject having liver disease an antibody or antibody reagent that inhibits WISP1.

In one embodiment of any aspect, the method further comprises, prior to (a), obtaining a biological sample from the subject.

In one embodiment of any aspect, the biological sample is a blood sample, tissue, buffy coat, serum, or tissue.

Yet another aspect described herein provides a method for treating or preventing a liver disease comprising administering to a subject in need thereof an agent that inhibits WISP1.

In one embodiment of any aspect, the agent that inhibits WISP1 is selected from the group consisting of: a small molecule, an antibody or antibody reagent, a peptide, a genome editing system, a viral vector, a miRNA, a lncRNA, a mRNA and a siRNA. In one embodiment of any aspect, the microRNA is miRNA15a or miRNA412.

In one embodiment of any aspect, the agent is administered by direct injection, subcutaneous injection, muscular injection, oral, transdermal or nasal administration.

Another aspect described herein provides a composition comprising an agent that inhibits WISP1 and a pharmaceutically acceptable carrier.

Another aspect described herein provides a method of treating a liver disease in a subject, comprising (a) detecting the level of WISP1 and/or Acta2 in a biological sample of a subject; (b) comparing the measurement of (a) to a reference level; (c) identifying a subject with increased WISP1 and/or Acta2 in (a) as compared to a reference level as having a liver disease; and (d) administering to the subject having liver disease an agent that inhibits WISP1.

Yet another aspect described herein provides a method of generating an engineered hepatic stellate cell, or population thereof, that expresses an agent that inhibits WISP1 comprising contacting the cell with an agent that inhibits WISP1, and culturing the cell for a sufficient time to allow for expression of the agent.

In one embodiment of any aspect, wherein the cell is quiescent.

In one embodiment of any aspect, the contacting comprises contacting the cell with an agent or a vector that encodes the agent. In one embodiment of any aspect, the contacting comprises transduction, nucleofection, electroporation, direct injection, and/or transfection.

Another aspect described herein provides a cell line comprising hepatic stellate cells generated by any of the methods described herein.

Another aspect described herein provides a pharmaceutical composition comprising a hepatic stellate cell, or population thereof, generated by any of the methods described herein, and a pharmaceutically acceptable carrier.

Another aspect described herein provides a method of treating or preventing a liver disease comprising administering to a subject in need thereof the cells generated by any of the methods described herein, any of the generated cells described herein, or any of the pharmaceutical composition comprising a generated cell described herein.

Another aspect described herein provides a method of reducing fibrosis in a subject, the method comprising: administering to a subject in need thereof the cells generated by any of the methods described herein, any of the generated cells described herein, or any of the pharmaceutical composition comprising a generated cell described herein.

Another aspect provided herein is a method of treating a liver disease in a subject comprising (a) receiving the results of an assay that identifies a subject as having increased WISP1 and/or Acta2 levels (e.g., mRNA, miRNA, protein levels, etc.) as compared to a reference level as having a liver disease; and (b) administering to the subject having liver disease an antibody or antibody reagent that inhibits WISP1.

Another aspect provided herein is a method of treating a liver disease in a subject comprising (a) receiving the results of an assay that identifies a subject as having increased WISP1 and/or Acta2 levels as compared to a reference level as having a liver disease; and (b) administering to the subject having liver disease an agent reagent that inhibits WISP1.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in immunology and molecular biology can be found in The Merck Manual of Diagnosis and Therapy, 19th Edition, published by Merck Sharp & Dohme Corp., 2011 (ISBN 978-O-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with liver disease, e.g., hepatic fibrosis. The term “treating” includes reducing or alleviating at least one adverse effect or symptom of liver disease, for example, jaundice, variceal bleeding, reduction in fibrosis, scarring and ascites. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing or reversal of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein “preventing” or “prevention” refers to any methodology where the disease state does not occur due to the actions of the methodology (such as, for example, administration of a composition as described herein). In one aspect, it is understood that prevention can also mean that the disease is not established to the extent that occurs in untreated controls. Accordingly, prevention of a disease encompasses a reduction in the likelihood that a subject can develop the disease, relative to an untreated subject (e.g. a subject who is not treated with the methods or compositions described herein).

As used herein, the terms “administering,” and “injecting” are used interchangeably in the context of the placement of cells, e.g., a hepatic stellate cells or an agent described herein, into a subject, by a method or route which results in at least partial localization of the introduced cells or agent at a desired site, such as the liver or a region thereof, such that a desired effect(s) is produced (e.g., decreased WISP1 level or activity). The agent or cells described herein can be administered by any appropriate route which results in delivery to a desired location in the subject where at least a portion of the delivered agent, cells, or components of the cells remain viable. The period of viability of the cells after administration to a subject can be as short as a few hours, e.g., twenty-four hours, to a few days, to as long as several years, i.e., long-term. In some embodiments, the term “administering” refers to the administration of a pharmaceutical composition comprising one or more agents or cells. The administering can be done by direct injection (e.g., directly administered to a target cell), subcutaneous injection, muscular injection, oral, or nasal delivery to the subject in need thereof. Administering can be local or systemic.

The terms “patient”, “subject” and “individual” are used interchangeably herein, and refer to an animal, particularly a human, to whom treatment, including prophylactic treatment is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. In another embodiment, the subject is a domesticated animal including companion animals (e.g., dogs, cats, rats, guinea pigs, hamsters etc.). A subject can have previously received a treatment for a liver disease, or has never received treatment for a liver disease. A subject can have previously been diagnosed with having a liver disease, or has never been diagnosed with a liver disease.

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder. For example, inhibiting WISP1 is inhibiting WISP1 activity or reducing WISP1 protein levels.

The terms “increased,” “increase” or “enhance” or “activate” are all used herein to generally mean an increase of a property, level, or other parameter by a statistically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

As used herein, a “reference level” refers to a normal, otherwise unaffected cell population or tissue (e.g., a biological sample obtained from a healthy subject, or a biological sample obtained from the subject at a prior time point, e.g., a biological sample obtained from a patient prior to being diagnosed with a liver disease, or a biological sample that has not been contacted with an agent or composition disclosed herein).

As used herein, an “appropriate control” refers to an untreated, otherwise identical cell or population (e.g., a biological sample that was not contacted by an agent or composition described herein, or not contacted in the same manner, e.g., for a different duration, as compared to a non-control cell).

The term “pharmaceutically acceptable” can refer to compounds and compositions which can be administered to a subject (e.g., a mammal or a human) without undue toxicity.

As used herein, the term “pharmaceutically acceptable carrier” can include any material or substance that, when combined with an active ingredient, allows the ingredient to retain biological activity and is non-reactive with the subject's immune system. Examples include, but are not limited to, any of the standard pharmaceutical carriers such as a phosphate buffered saline solution, emulsions such as oil/water emulsion, and various types of wetting agents. The term “pharmaceutically acceptable carriers” excludes tissue culture media. Non limiting examples of pharmaceutical carriers include particle or polymer-based vehicles such as nanoparticles, microparticles, liposomes, polymer microspheres, or polymer-drug conjugates.

As used herein, the term “WNT1-inducible-signaling pathway protein 1” or “WISP1” or “CCN4” is a matricellular protein that is encoded by the WISP]gene that has many diverse cellular functions, including cell adhesion, migration, proliferation, differentiation, and survival. In the liver, WISP1 is secreted by hepatic stellate cells (HSCs) as they become activated toward myofibroblasts. Through the autocrine system, WISP1 also affects HSCs by accelerating activation and secretion of collagen to promote fibrosis. Sequences for WISP1, also known as CCN4, WISP1c, WISP1i, WISP1tc, WISP1-OT1, and WISP1-UT1, are known for a number of species, e.g., human WISP1 (NCBI Gene ID: 8840) polypeptide (e.g., NCBI Ref Sequence: NP_001191798.1) and mRNA (e.g., NCBI Ref Sequence: NM_001204869.1). WISP1 can refer to human WISP1, including naturally occurring variants, molecules, and alleles thereof. WISP1 refers to the mammalian WISP1 of, e.g., mouse, rat, rabbit, dog, cat, cow, horse, pig, and the like. The nucleic sequence of SEQ ID NO: 5 comprises the nucleic sequence which encodes WISP1.

As used herein, the term “WISP1 activity” refers to the cellular functions of WISP1, for example, WISP1 accelerates activation and secretion of collagen to promote fibrosis in HSCs, and attenuates p53-mediated apoptosis and WISP1 can inhibit TNF-induced cell death in other cell types. For example, an increase in WISP1 activity can refer to the increase in collagen deposition by a cell. WISP1 activity can refer to the induction of alpha smooth muscle actin expression or the expression of some pro-inflammatory cytokines, like IL-6.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The agent can be a molecule from one or more chemical classes, e.g., organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertion and other variants.

As used herein an “antibody” refers to IgG, IgM, IgA, IgD or IgE molecules or antigen-specific antibody fragments thereof (including, but not limited to, a Fab, F(ab′)₂, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria.

In another example, an antibody includes two heavy (H) chain variable regions and two light (L) chain variable regions. It should be noted that a V_{H} region (e.g. a portion of an immunglobin polypeptide is not the same as a V_{H} segment, which is described elsewhere herein). The V_{H} and V_{L} regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each V_{H} and V_{L} is typically composed of three CDRs and four FRs, arranged from amino-terminus to carboxy-terminus in the following order: FR1, CDR1, FR2, CDR2, FR3, CDR3, FR4. As used herein, the terms “protein” and “polypeptide” are used interchangeably herein to designate a series of amino acid residues, connected to each other by peptide bonds between the alpha-amino and carboxy groups of adjacent residues.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, nanobodies, humanized antibodies, chimeric antibodies, and the like. An antibody reagent can be an antibody fragment.

The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

As described herein, an “antigen” is a molecule that is bound by a binding site on an antibody. Typically, antigens are bound by antibody ligands and are capable of raising an antibody response in vivo. An antigen can be a polypeptide, protein, nucleic acid or other molecule or portion thereof. The term “antigenic determinant” refers to an epitope on the antigen recognized by an antigen-binding molecule, and more particularly, by the antigen-binding site of said molecule.

As used herein, the term “affinity” refers to the strength of an interaction, e.g. the binding of an antibody for an antigen and can be expressed quantitatively as a dissociation constant (K_{D}). Avidity is the measure of the strength of binding between an antigen-binding molecule (such as an antibody reagent described herein) and the pertinent antigen. Avidity is related to both the affinity between an antigenic determinant and its antigen binding site on the antigen-binding molecule, and the number of pertinent binding sites present on the antigen-binding molecule. Typically, antigen-binding proteins (such as an antibody reagent described herein) will bind to their cognate or specific antigen with a dissociation constant (K_{D} of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (KA) of 105 to 10¹² liter/moles or more, and preferably 10⁻⁷ to 10⁻¹² liter/moles or more and more preferably 10⁸ to 10¹² liter/moles). Any K_{D} value greater than 10⁻⁴ mol/liter (or any KA value lower than 10⁴ M⁻¹) is generally considered to indicate non-specific binding. The K_{D} for biological interactions which are considered meaningful (e.g. specific) are typically in the range of 10⁻¹⁰ M (0.1 nM) to 10⁻⁵ M (10000 nM). The stronger an interaction is, the lower is its K_{D}. Preferably, a binding site on an antibody reagent described herein will bind to the desired antigen with an affinity less than 500 nM, preferably less than 200 nM, more preferably less than 10 nM, such as less than 500 pM. Specific binding of an antibody reagent to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as other techniques as mentioned herein.

As used herein, the term “specific binding” or “specificity” refers to a chemical interaction between two molecules, compounds, cells and/or particles wherein the first entity binds to the second, target entity with greater specificity and affinity than it binds to a third entity which is a non-target. In some embodiments of any of the aspects, specific binding can refer to an affinity of the first entity for the second target entity which is at least 10 times, at least 50 times, at least 100 times, at least 500 times, at least 1000 times or greater than the affinity for the third nontarget entity. Accordingly, as used herein, “selectively binds” or “specifically binds” refers to the ability of an agent (e.g. an antibody reagent) described herein to bind to a target, such a peptide comprising, e.g. the amino acid sequence of a given antigen, with a K_{D} 10⁻⁵ M (10000 nM) or less, e.g., 10⁻⁶ M or less, 10⁻⁷ M or less, 10⁻⁸ M or less, 10⁻⁹ M or less, 10⁻¹⁰M or less, 10¹¹ M or less, or 10⁻¹² M or less. For example, if an agent described herein binds to a first peptide comprising the antigen with a K_{D} of 10⁻⁵ M or lower, but not to another randomly selected peptide, then the agent is said to specifically bind the first peptide. Specific binding can be influenced by, for example, the affinity and avidity of the agent and the concentration of the agent. The person of ordinary skill in the art can determine appropriate conditions under which an agent selectively bind the targets using any suitable methods, such as titration of an agent in a suitable cell and/or a peptide binding assay.

The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “contacting” when used in reference to a cell or organ, encompasses both introducing an agent, surface, hormone, etc. to the cell in a manner that permits physical contact of the cell with the agent, surface, hormone etc., and introducing an element, such as a genetic construct or vector, that permits the expression of an agent, such as a miRNA, polypeptide, or other expression product in the cell. It should be understood that a cell genetically modified to express an agent, is “contacted” with the agent, as are the cell's progeny that express the agent.

The term “statistically significant” or “significantly” refers to statistical significance and generally means a two standard deviation (2SD) or greater difference.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Further, unless otherwise required by context, singular terms shall include pluralities and plural terms shall include the singular.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a composition of this invention, dose, time, temperature, and the like, is meant to encompass variations of ±20%, ±10%, ±5%, ±1%, ±0.5%, or even ±0.1% of the specified amount. Other than in the operating examples, or where otherwise indicated, all numbers expressing quantities of ingredients or reaction conditions used herein should be understood as modified in all instances by the term “about.” The term “about” when used in connection with percentages can mean±1%.

Unless otherwise explained, all technical and scientific terms used herein have the same meaning as commonly understood by one of ordinary skill in the art to which this disclosure belongs.

It should be understood that this disclosure is not limited to the particular methodology, protocols, and reagents, etc., described herein and as such may vary. The terminology used herein is for the purpose of describing particular embodiments only, and is not intended to limit the scope of the present disclosure, which is defined solely by the claims.

All patents and other publications identified are expressly incorporated herein by reference for the purpose of describing and disclosing, for example, the methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

### BRIEF DESCRIPTION OF THE DRAWINGS

FIG. 1 shows schematic of research plan: Aims 1 probes miR-15a's and miR-412's mechanism and function, Aim 2 tests their treatment potential, and Aim 3 elucidates the function of miR-15a's known target WISP1. HSC is a term for Hepatic stellate cell.

FIG. 2 shows schematic of flow of the experiment that screened a full genome microRNA mimic library to identify candidates that revert activated hepatic stellate cells toward quiescence.

FIG. 3 shows activated mouse hepatic stellate cells (HSCs) reverted toward quiescence, demonstrated by re-formation of Nile Red stain-positive lipid droplets, when miR-15a or miR-412 were overexpressed (top row). FIG. 3 also shows activated human HSCs reverted toward quiescence, demonstrated by re-formation of lipid droplets, when human orthologues of miR-15a or miR-412 were overexpressed (bottom row).

FIGS. 4A and 4B show miR-15a or miR-412 delivery to activated hepatic stellate cells (HSCs) caused changes in morphology toward that of quiescence. FIG. 4A shows the size of the HSCs decreased by 10-100 folds as all of the photos were taken with the same magnification (same scale bar for all panels). FIG. 4B. shows that forced expression of miR-15a or miR-412 in initially activated HSCs downregulated alpha smooth muscle actin (Acta2) and alpha-1 type I collagen (Colla1) measured with qPCR. Data are presented as mean+/−SD.

FIG. 5 shows that miR-15a or miR-412-transfected hepatic stellate cells (HSCs) have a functional phenotype. Activated HSCs treated with miR-15a or miR-412 did not cause steatosis in cocultured hepatocytes. Activated HSCs treated with negative control microRNA induced steatosis in cocultured hepatocytes. qHSC, quiescent hepatic stellate cell; Ac-HSC, activated hepatic stellate cell; miR-Neg, microRNA negative control.

FIG. 6 shows that HepG2 cells cocultured with activated human HSCs that received miR-15a or miR-412 have decreased expression of pro-inflammatory cytokines (left panel). Huh7 cells cocultured with activated human HSCs that received miR-15a or miR-412 have decreased expression of some pro-inflammatory cytokines (right panel). Data are presented as mean±1− SD.

FIG. 7 shows endogenous miR-15a and miR-412 had decreased expression levels in activated primary HSCs compared to quiescent HSCs, although not significantly for miR-15a. Data are presented as mean +1− SD.

FIG. 8 shows schematic of schedule for CCl₄ challenge and cell therapy injection (diagram). Top row: Quiescent-like HSCs reprogrammed with miR-15a or miR-412 injected into the spleen grafted on to the liver, evidenced by the liver producing the GFP signal built into the vector driving the miRNA expression. Middle row: CCl₄ challenged mice that received quiescent-like HSCs had decreased hepatic apoptosis and inflammation demonstrated with H&E stain. Bottom row: CCl₄ challenged mice that received quiescent-like HSCs had decreased hepatic fibrosis stained with Sirius Red. The relative levels of fibrosis were quantified. CCl₄, CCl₄ gavage without injecting HSCs; HSC Control, CCl₄ gavage and injecting HSCs with empty GFP-vector; HSC miR-15a, CCl₄ gavage and injecting HSCs with miR-15a-GFP-vector; HSC miR-412, CCl₄ gavage and injecting HSCs with miR-412-GFP-vector.

FIG. 9 shows cell therapy with reprogrammed quiescent-like HSCs caused mice challenged with CCl₄ to have decreased expression of alpha-1 type I collagen (Colla1) in the whole liver measured by qPCR. Data are presented as mean +1− SD.

FIG. 10 shows hepatic stellate cells in human primary biliary cholangitis expressed WISP1 which co-localized with alpha smooth muscle actin (Acta2).

FIG. 11 shows that miR-15a mimic co-transfected with a reporter containing either one of the two predicted WISP1 target sequences decreased luciferase expression while it did not affect the reporters containing mutated sequences, indicating that miR-15a binds to both WISP1 target sequences. Data are presented as mean +1− SD (**P<0.01; ***P<0.001).

FIG. 12 shows that quiescent HSCs in low and high magnification (left column). The high magnification view clearly shows several lipid droplets that fluoresce green with BODIPY stain (inset). Right column: Activated HSCs in low and high magnification. Activated cells are much larger, lack lipid droplets, and do not stain with BODIPY (inset). HSC, hepatic stellate cell.

FIG. 13 shows that comparison of mice fed with standard diet (left column) and with CDAHFD (right column). Mice fed with CDAHFD developed early NASH in three weeks, showing increased body size, steatotic liver grossly and microscopically, and trace fibrosis by Sirius Red stain. CDAHFD, choline-deficient L-amino acid defined high fat diet.

FIG. 14 shows the schematic of the experiment showing coculture of healthy hepatocytes (Hep) with either control hepatic stellate cells (HSC) from unchallenged mice or HSCs from CDAHFD challenged NASH mice. Top row: Hepatocytes cocultured with HSCs from control mice showed very few lipid droplets stained with BODIPY. Bottom row: Hepatocytes cocultured with HSCs from NASH mice showed significantly more lipid droplets. CDAHFD, choline-deficient L-amino acid defined high fat diet.

FIG. 15 shows hepatocytes cocultured with hepatic stellate cells (HSC) from NASH mice expressed higher levels of several inflammatory cytokines and chemoattractants than when they were cocultured with control HSCs, measured with qPCR. Data are presented as mean+/−SD.

FIG. 16 shows induction of fatty accumulation in hepatocytes (Hep) can be achieved when the conditioned media from NASH-hepatic stellate cells (HSC) is applied to normal hepatocytes. Top row: When quiescent HSC (qHSC) media was applied to healthy hepatocytes, steatosis was not induced. Bottom row: NASH-HSC media induced steatosis in initially healthy hepatocytes. Lipid droplets stained with BODIPY.

FIG. 17 shows that delivering miR-15a or miR-412 into activated hepatic stellate cells (AcHSC) induced re-formation of lipid droplets that are retinoid positive evidenced by fluorescence under ultraviolet light, consistent with those in quiescent hepatic stellate cells (qHSC).

FIG. 18 shows RNA sequencing data analyzed with multi-dimensional scaling demonstrated that the quiescent-like HSCs that received miR-15a or miR-412 had a global transcriptional profile 4D-50% closer to quiescent HSCs than activated cells. HSC, hepatic stellate cell.

FIG. 19 shows that miR-15a or miR-412-transfected hepatic stellate cells (HSC) have a functional phenotype. Top row: Activated HSCs treated with miR-15a or miR-412 did not cause steatosis in cocultured hepatocytes. Activated HSCs treated with negative control microRNA induced steatosis in cocultured hepatocytes. qHSC, quiescent hepatic stellate cell: Ac-HSC, activated hepatic stellate cell; miR-Neg, microRNA negative control. Bottom row: HSCs harvested from CDAHFD model of NASH induced steatosis in cocultured hepatocytes. These same HSCs infected with lentivirus expressing miR-15a or miR-412 bet their ability to induce steatosis in adjacent hepatocytes.

FIG. 20 shows overlaying the set of genes with decreased mRNA level in hepatic stellate cells after receiving either miR-15a or miR-412 with the set of potential direct targets based on a prediction algorithm produced the target candidate sets that are more likely to include true miRNA targets. The genes in the target candidate sets were further filtered by choosing those that are part of the Tgf-beta or Pdgf signaling pathway.

FIG. 21 shows CRISPR technology can be used in primary hepatic stellate cells. The viability of using this technology on primary cells was verified by delivering long noncoding RNA Digit deletion vectors. Homozygous knock-in is confirmed with the appearance of new PCR bands, one allele with puromycin construct and another with neomycin construct. Ctl, control construct; KI, knock-in construct.

FIG. 22 shows a protein blot of >100 cytokines, chemokines, and extracellular matrix proteins showed that HSCs from mice with CDAHFD induced NASH up-secreted WISP1 compared to those from healthy mice. CM, conditioned media.

FIG. 23 shows activated hepatic stellate cells (AcHSC) expressed WISP1 nearly 30 times higher than quiescent hepatic stellate cells (qHSC). Hepatocytes (Hep) also expressed WISP1, but significantly less than activated hepatic stellate cells. Lv, whole liver. Data are presented as mean+/−SD.

FIG. 24 shows conditioned media from hepatic stellate cells overexpressing WISP1 induced steatosis in hepatocytes harvested from healthy mice. CM, conditioned media.

FIG. 25 is a schematic showing WISP1 is involved in human disease.

FIG. 26 is a schematic showing WISP 1 is a member of the CCN family of secreted matricellular proteins.

FIG. 27 is a schematic showing that hepatic stellate cells are key drivers of liver fibrosis.

FIG. 28 is a schematic showing hepatic stellate cell (HSC) activation.

FIGS. 29A and 29B show WISP1 is highly upregulated in activated HSCs and is a direct target of miR-15a. (FIG. 29A) WISP1 secreted by activated hepatic stellate cells in NASH mice. (FIG. 29B) mrR-15a mimic co-transfected with a reported containing predicted WISP1 target sequences decreased luciferase expression.

FIGS. 30A-30C show WISP1 and Yap 1 mutually activate each other. (FIG. 30A) WISP1 and YAP mRNA increased by rc-WISP1 treatment in qHSC. (FIG. 30B) YAP1 activation induced by rc-WISP1 treatment in qHSC. (FIG. 30C) WISP1 expression increased by YAP1 overexpression in reprogrammed HSC by miRNA.

FIGS. 31A and 31B show existing literature supporting role of WISP inhibition in liver and lung fibrosis. (FIG. 31A) Anti-WISP1 mAb attenuated CCl₄-induced liver fibrosis. (FIG. 31B) Anti-WISP1 mAb attenuated bleomycin-induced lung fibrosis

FIGS. 32A and 32B show WISP1 neutralizing antibodies attenuate biliary fibrosis. (FIG. 32A) In vivo treatment with neutralizing antibody to WISP1 decreased bile duct ligation (BDL) induced liver fibrosis. (FIG. 32B) Collagen expression in indicated conditions.

FIGS. 33A and 33B shows WISP1 is secreted in common and rare human fibrotic liver diseases. (FIG. 33A) WISP1 upregulated in HSCs of NASH. (FIG. 33B) WISP1 upregulated in HSCs of several rare fibrotic liver diseases.

FIGS. 34A and 34B show WISP1 is a novel secreted fibrotic driver for liver inflammation and fibrosis. (FIG. 34A) Activated HSCs from NAFLD mice secrete WISP1. (FIG. 34B) WISP1 overexpressing-HSCs induce steatosis in healthy co-cultured primary hepatocytes (BODIPY stain).

FIG. 35 shows Rc WISP1 treatment accelerates HSC proliferation measured by ki67.

FIG. 36 shows sequence alignment of human and mouse miR-15a and miR-412.

FIGS. 37A and 37B shows WISP1 expressed by HSCs have self-activating autocrine and pro-steatotic paracrine effects. FIG. 37A shows that HSC proliferation and activation induced by recombinant WISP1 treatment in mice. FIG. 37B shows immunohistochemistry of Control and RcWisp1.

FIGS. 38A-38F show that Rc WISP1 and Wisp IgG treat NASH-HSCs from choline deficient, L-amino acid defined, high fat diet model-induced steatosis (CDAHFD) and prevent fibrosis. 1 μg/ml WISP1 antibody was incubated with conditioned media for 1 hour at 37° C. before applying to HSCs. FIG. 38A shows the timeline of treatment and sampling. FIG. 38B shows cell number of control, Rc WISP1 treated, and Ab WISP1 treated cells. FIG. 38C shows Acta2 mRNA expression of qHSCs, control, Rc WISP1 treated, and Ab WISP1 treated cells. FIG. 38D shows Colla1 mRNA expression of qHSCs, control, Rc WISP1 treated, and Ab WISP1 treated cells. FIG. 38E shows immunohistochemistry of control, Rc WISP1 treated, and Ab WISP1 treated cells. FIG. 38F shows the quantification of BODIPY stained area fraction of control, Rc WISP1 treated, and Ab WISP1 treated cells.

FIGS. 39A and 39B demonstrate that WISP1 modulates HSC migration. FIG. 39A shows images of HSC migration in the presence and absence of Re-WISP1 and WISP1 IgG. FIG. 39B shows relative wound area of HSCs treated with Re-WISP1 and WISP1 IgG compared to control HSCs. HSCs treated with WISP1 IgG exhibited a relative wound area that was similar to control HSCs within 24 hours, confirming that WISP1 IgG can prevent HSC activation and migration.

### DETAILED DESCRIPTION

Liver disease (e.g., primary biliary cholangitis (PBC)), can lead to progressive destruction of intrahepatic bile ducts, cholestasis, periportal inflammation, and eventually biliary fibrosis that ends as cirrhotic end-stage liver disease. Liver diseases, such as PBC, can manifest as an autoimmune disorder with unclear pathogenesis although both genetic and environmental factors likely cause the liver disease.

WISP1 is a protein that is secreted by hepatic stellate cells (HSCs) of the liver as they become activated toward myofibroblasts. Through the autocrine system, WISP1 also affects HSCs by accelerating activation and secretion of collagen to promote fibrosis. Among fibroblasts in liver disease (e.g. primary biliary cholangitis, autoimmune hepatitis, alpha1-antitrypsin deficiency, non-human diseases, myoalcholic steatohepatitis, and scleroderma) highly express WISP1. Therefore, indicating that inhibiting WISP1 is a strategy to treat diseases of the liver.

Hepatic stellate cells are fat-storing pericytes of the liver that are found in the perisinusoidal space (also known as the space of Disse). Quiescent HSCs are distinguished by the presence of lipid droplets within the cytoplasm. When the liver is damaged, stellate cells can become activated. Activated HSCs proliferate and have reduced lipid droplets within the cytoplasm. Activated HSCs become pro-fibrotic myofibroblasts that secrete collagen and mediators that promote scar formation.

The methods described herein show that WISP1 is upregulated in HSCs. When WISP1 is inhibited by microRNAs, miR-15a and miR-412, this inhibition independently induces quiescence in activated hepatic stellate cells (HSCs), the cell type that plays a central role in fibrotic progression of the liver. Furthermore, miR-15a directly targets WISP1 to repress the protein's pro-fibrotic function in activated HSCs. By promoting HSC quiescence using microRNAs and their target, the WISP1 inhibitor is a useful therapy to control progressive hepatic fibrosis in liver diseases such as PBC.

Treating and/or Preventing Liver Disease

The methods and compositions described herein are used to treat and/or prevent a liver disease in a subject. Exemplary liver diseases include, but are not limited to, Alagille Syndrome; Alcohol-Related Liver Disease; Alpha-1 Antitrypsin Deficiency; Autoimmune Hepatitis; Benign Liver Tumors; Biliary Atresia; Cirrhosis; Crigler-Najjar Syndrome; Galactosemia; Gilbert Syndrome; Hemochromatosis; Hepatic Encephalopathy; Hepatitis A; Hepatitis B; Hepatitis C; Hepatorenal Syndrome; Intrahepatic Cholestasis of Pregnancy (ICP); Lysosomal Acid Lipase Deficiency (LAL-D); Liver Cysts; Liver Cancer; Newborn Jaundice; Non-Alcoholic Fatty Liver Disease; Non-Alcoholic Steatohepatitis; Primary Biliary Cholangitis (PBC); Primary Sclerosing Cholangitis (PSC); Progressive Familial Intrahepatic Cholestasis (PFIC); Reye Syndrome; Type I Glycogen Storage Disease; scleroderma; and Wilson Disease. In one embodiment, the liver disease is primary biliary cholangitis, autoimmune hepatitis, alpha 1 antitrypsin deficiency, non-alcoholic steatohepatitis, and scleroderma.

In one embodiment, the methods described herein are used to treat a subject having liver failure, e.g., failure of hepatic synthetic and metabolic function. In one embodiment, the methods described herein are used to treat a subject having fulminant or severe acute hepatic failure (e.g., liver failure with encephalopathy developing over <8 weeks in previously healthy individuals); Hyperacute liver failure (e.g., liver failure with encephalopathy developing over <14 days in previously healthy individuals); Acute liver failure (e.g., liver failure with encephalopathy developing over <26 weeks in previously healthy individuals); Chronic liver failure (e.g., liver failure without encephalopathy); or Acute on chronic liver failure (e.g., chronic liver failure with the development of encephalopathy). A skilled practitioner can assess the severity of the liver disease (e.g., liver failure) using standard methods, for example, Child-Pugh Score (which is a composite of Total bilirubin, albumin, INR, ascites and hepatic encephalopathy), MELD Score (which uses the serum bilirubin, creatinine and INR), or PELD Score (similar to MELD but for pediatric patients), or METAVIR Score (which assesses the level of fibrosis is a sample. METAVIR

In one embodiment, the methods described herein are used to treat a subject having liver fibrosis. Liver fibrosis can be diagnosed by a skilled clinician using, e.g., METAVIR Score. The METAVIR Score provides two scores, a fibrosis score and an activity score. The fibrosis score is used to describe the amount of inflammation (the intensity of inflammation/breakdown of tissue) in the liver, e.g., F0: No fibrosis; F1: Portal fibrosis without septa; F2: Portal fibrosis with few septa; F3: Numerous septa without cirrhosis; F4: Cirrhosis. The activity score is a prediction about how rapidly the degree of fibrosis is progressing, e.g., A0: No activity; A1: Mild activity; A2: Moderate activity; and A3: Severe activity. In one embodiment, a subject treated with methods described herein has a METAVIR Score of F1, F2, F3, or F4 and/or A1, A2, A3.

In another aspect, described herein is a method for treating or preventing a liver disease, the method comprises: administering to a subject in need thereof an antibody or antibody reagent that inhibits WISP1.

In one aspect, described herein is a method for treating or preventing a liver disease comprising administering to a subject in need thereof an agent that inhibits WISP1.

In various embodiments, WISP1 is inhibited in a target cell. In one embodiment, the target cell is a liver cell that highly expresses WISP1 and results in a disease state, e.g., a liver disease. For example, a liver cell that expresses increased levels of WISP1 as compared to an appropriate control, e.g., a healthy, non-diseased liver cell. One skilled in the art can assess the mRNA or protein levels of WISP1 in a cell using PCR-based assays or western-blotting, respectively, using standard techniques.

In another embodiment, a target cell is a hepatic stellate cell (HSC). HSCs are pericytes found in the perisinusoidal space of the liver. One skilled in the art can determine if a cell is an HSC using, e.g., selective staining for gold chloride or visualization of lipid droplets in the cytoplasm. In one embodiment, the HSC is quiescent. A quiescent HSC can be identified by one skilled in the art by the presence of lipid droplets in the cytoplasm in a non-proliferating cell. An active HSC can be identified by one skilled in the art by assessing proliferation of the cell, reduced lipid droplets within the cytoplasm, and/or secretion of collagen and mediators that promote scar formation.

In one embodiment, a target cell is a fibroblast, e.g., a liver fibroblast. In one embodiment, a target cell is a myofibroblast. A myofibroblast cell has characteristics of both a fibroblast cell and a smooth muscle cell. Fibroblasts and myofibroblasts can readily be identified by one skilled in the art, e.g., by selecting for fibroblast or fibroblast and smooth muscle cell markers, respectively, e.g., via microscopy.

In one embodiment, a target cell is a mammalian cell, preferably a human cell.

In another aspect, described herein is a method of treating a liver disease comprising (a) measuring the level of WISP1 and/or Yap, Colla1, Acta2 in a biological sample of a subject; (b) comparing the measurement of (a) to a reference level; (c) identifying a subject with increased WISP1 and/or Yap, Colla1, Acta2 in (a) as compared to a reference level as having a liver disease; and (d) administering to the subject having liver disease an antibody or antibody reagent that inhibits WISP1.

In yet another aspect, described herein is a method of treating a liver disease comprising (a) measuring the level of WISP1 and/or Yap, Colla1, Acta2 in a biological sample of a subject; (b) comparing the measurement of (a) to a reference level; (c) identifying a subject with increased WISP1 and/or Yap, Colla1, Acta2 levels in (a) as compared to a reference level as having a liver disease; and (d) administering to the subject having liver disease an agent that inhibits WISP1.

Assays for measuring WISP1 and/or Yap, Colla1, Acta2 levels include, but are not limited to, PCR-based assays to assess WISP1 and/or Yap, Colla1, Acta2 mRNA levels, or western-blotting to assess WISP1 and/or Yap, Colla1, Acta2 protein levels. In one embodiment, WISP1 and/or Yap, Colla1, Acta2 levels are increased by at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more, or at least one 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold or more as compared to a reference level. In one embodiment, WISP1 and/or Yap, Colla1, Acta2 levels are WISP1 and/or Yap, Colla1, Acta2 mRNA levels. In another embodiment, WISP1 and/or Yap, Colla1, Acta2 levels are WISP1 and/or Yap, Colla1, Acta2 protein levels.

As used herein, a “reference level” refers to an otherwise identical biological sample of a healthy subject, e.g., a subject that does not have a liver disease.

In one embodiment, the method described herein further comprises, prior to (a) obtaining a biological sample from the subject. As used herein, biological sample refers to a blood sample, a tissue sample, a buffy coat sample (e.g., the fraction of an anticoagulated blood sample that contains a high level of white blood cells and platelets following centrifugation, a serum sample, or a liver biopsy sample. A biological sample can be obtained using common, appropriate techniques known in the art. For example, tissue samples can be obtained via biopsy, and blood samples can be obtained from finger prick or intravenous blood draw.

In various embodiments, biological samples are taken from a subject that has previously or not previously been diagnosed with a liver disease. In another embodiment, the biological sample is taken from a subject that is suspected of having a liver disease, e.g., a subject how has at least one risk factor for liver disease, e.g., increased alcohol intake as compared to normal intake.

Another aspect provided herein is a method of treating a liver disease in a subject comprising (a) receiving the results of an assay that identifies a subject as having increased WISP1 and/or Acta2 levels as compared to a reference level as having a liver disease; and (b) administering to the subject having liver disease an antibody or antibody reagent that inhibits WISP1.

Another aspect provided herein is a method of treating a liver disease in a subject comprising (a) receiving the results of an assay that identifies a subject as having increased WISP1, Yap, Colla1 and/or Acta2 levels as compared to a reference level as having a liver disease; and (b) administering to the subject having liver disease an agent reagent that inhibits WISP1.

Assays for measuring WISP1, Yap (NCBI Gene ID 10413), Colla1 (NCBI Gene ID 1277) and/or Acta2 (NCBI Gene ID 59) levels include, but are not limited to, PCR-based assays to assess WISP1 and/or Yap, Colla1, Acta2 mRNA levels, or western-blotting to assess WISP1 and/or Yap, Colla1, Acta2 protein levels. An assay (e.g., a PCR-based assay to assess WISP1 and/or Yap, Colla1, Acta2 mRNA levels, or western-blotting to assess WISP1 and/or Yap, Colla1, Acta2 protein levels) can be performed by the skilled practitioner administering the agent (e.g., an antibody or antibody reagent) that inhibits WISP1. Alternatively, the assay can be performed by another individual (i.e., not by the practitioner administering the agent (e.g., an antibody or antibody reagent) that inhibits WISP1). Results of an assay can be received by any means, e.g., via mail courier, telephonic transmission (e.g., facsimile), electronic transmission (e.g., electronic medical records, electronic mail (email), or the like. In one embodiment, treatment is administered to the subject at any time (e.g., at least 1 second, 1 min, 1 hour, 1 day, 1 week, 1 month, 1 year, or longer) after receiving the results of the assay.

In one embodiment, WISP1 and/or Yap, Colla1, Acta2 levels are increased by at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more, or at least one 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold or more as compared to a reference level. In one embodiment, WISP1 and/or Yap, Colla1, Acta2 levels are WISP1 and/or Yap, Colla1, Acta2 mRNA levels. In another embodiment, WISP1 and/or Yap, Colla1, Acta2 levels are WISP1 and/or Yap, Colla1, Acta2 protein levels.

In another aspect, described herein is a method for treating or preventing a liver disease comprising administering to a subject in need thereof any of the cells generated using methods described herein, or any of the pharmaceutical composition comprising cells generated using methods described herein.

In another aspect provided herein, any of the antibodies or antibody reagents, agents, cells generated using methods described herein, compositions, of pharmaceutical compositions described herein can be used to treat or reduce fibrosis of the liver in a subject. In one aspect described herein, is a method for reducing fibrosis, e.g., liver fibrosis, comprising administering to a subject in need thereof any of the cells generated using methods described herein, or any of the pharmaceutical composition comprising cells generated using methods described herein.

In one embodiment, the subject has previously been diagnosed with having a liver disease. In another embodiment, the subject is diagnosed with a liver disease prior to the administering of the agent. One skilled in the art can diagnose a subject of having a liver disease, e.g., using standard techniques in the art. For example, blood tests referred to as liver function tests; non-invasive imaging such as CT-scan, ultrasound, or MRI scan; or tissue biopsy. Liver function tests refer to blood tests that assess levels of liver-specific enzymes, e.g., alanine transaminase (ALT), aspartate aminotransferase (AST), alkaline phosphatase (ALP), albumin, or bilirubin. Even mildly elevated levels of ALT, AST, ALP, albumin, or bilirubin can indicate a liver disease. Further tests for assessing liver disease are described herein (e.g., Child-Pugh Score. METAVIR Score, and PELD and MELD Score.

In another embodiment, the agents described herein inhibit a WISP1 splice variant in a target cell. Exemplary WISP1 splice variants include, but are not limited to, WISP1v, WISP1vx, and WISP1delta exon 3-4.

In another embodiment, inhibiting WISP1 is inhibiting WISP1 activity. The WISP1 activity can be any currently known, or yet to be discovered activity of function of the WISP1 gene or gene product. For example, WISP1 accelerates activation and secretion of collagen to promote fibrosis in HSCs, and attenuates p53-mediated apoptosis and inhibit TNF-induced cell death in other cell types. In another embodiment, the WISP1 activity is inhibited by at least 5%, 10%, 15%, 20%, 25%, 30%, 35%, 40%, 45%, 50%, 55%, 60%, 65%, 70%, 75%, 80%, 85%, 90%, 95%, 99%, or more as compared to an appropriate control.

As used herein, an “appropriate control” refers to the level of WISP1 activity is an otherwise identical sample that is not contacted by an agent or composition described herein, or is the level of WISP1 activity in a subject prior to administration of an agent or composition. Furthermore, an appropriate control can be the level of WISP1 activity in a healthy subject, e.g., an individual that does not have a liver disease. One skilled in the art can determine the activity of WISP1 using functional readouts of WISP1's activity, for example, by measuring/assessing/quantifying the activation and secretion of collagen in HSCs.

In another embodiment, inhibiting WISP1 is inhibiting WISP1 levels in the cell, e.g, gene expression levels or gene product levels. In another embodiment, WISP1 levels are inhibited by at least 5%, 10%, 15%, 20%, 25%, 30%, 35%, 40%, 45%, 50%, 55%, 60%, 65%, 70%, 75%, 80%, 85%, 90%, 95%, 99%, or more as compared to an appropriate control. As used herein, an “appropriate control” would be the level of WISP1 is an otherwise identical sample that is not contacted by an agent or composition described herein, or is the level of WISP1 in a subject prior to administration of an agent or composition. Further, an appropriate control can be the level of WISP1 in a healthy subject, e.g., an individual that does not have a liver disease. One skilled in the art can determine the activity of WISP1 using functional readouts of WISP1's activity, for example, by measuring/assessing the activation and secretion of collagen in HSCs. One skilled in the art can assess/measure the protein and mRNA levels of WISP1, e.g., using western blotting or PCR-based assays, respectively.

### Agents

In one aspect, an agent that inhibits WISP1 is administered to a subject having or at risk of having a liver disease. In one embodiment, the agent is a small molecule, an antibody, a peptide, a genome editing system, a viral vector, a miRNA, and a siRNA.

An agent described herein is considered effective for inhibiting WISP1 if, for example, upon administration, it inhibits the presence, amount, activity and/or level of WISP1 in the cell.

An agent can inhibit e.g., the transcription, or the translation of WISP1 in the cell. An agent can inhibit the activity or alter the activity (e.g., such that the activity no longer occurs, no longer occurs properly (e.g., as compared to wild-type WISP1 activity), or occurs at a reduced rate) of WISP1 in the cell (e.g., WISP1's expression).

In one embodiment, the agent excludes miRNA 412 and miRNA 15a. In another embodiment, the agent excludes any miRNA 412 or miRNA 15a mimics.

The agent may function directly in the form in which it is administered. Alternatively, the agent can be modified or utilized intracellularly to produce something which inhibits WISP1, such as introduction of a nucleic acid sequence into the cell and its transcription resulting in the production of the nucleic acid and/or protein inhibitor of WISP1 within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be identified from a library of diverse compounds.

In various embodiments, the agent is a small molecule that inhibits WISP1. As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Methods for screening small molecules are known in the art and can be used to identify a small molecule that is efficient at, for example, inhibiting WISP1 activity or levels, given the desired target (e.g., WISP1 polypeptide).

One aspect provided herein is a composition comprising any of agents that inhibit WISP1 described herein. In one embodiment, the composition further comprises a pharmaceutically acceptable carrier. In one embodiment, the composition is a pharmaceutical composition.

Polypeptides that Inhibit WISP1

In one embodiments, described herein is a method treating or preventing a liver disease comprising administering to a subject in need thereof a polypeptide or a nucleic acid encoding such polypeptide that inhibits WISP1 in a target cell.

The term “WISP1-binding polypeptide” refers to a polypeptide that specifically binds to a desired antigen of interest (e.g., WISP1 polypeptide) and that is an Ig-like protein comprising one or more of the antigen binding domains described herein linked to a linker or an immunoglobulin constant domain. A binding protein can be, in some embodiments, a dual variable domain (DVD-Ig) binding protein. A “linker polypeptide” comprises two or more amino acid residues joined by peptide bonds and are used to link one or more antigen binding portions. Such linker polypeptides are well known in the art (see e.g., Holliger et al. (1993) Proc. Natl. Acad. Sci. USA 90: 6444-6448; Poljak (1994) Structure 2: 1121-1123). An immunoglobulin constant domain refers to a heavy or light chain constant domain. Human IgG heavy chain and light chain constant domain amino acid sequences are known in the art, (e.g., see SEQ ID NO: 197, 198, 199 and 200 of US Application 2016/0200813, which is incorporated herein in its entirety by reference for representative examples).

In some embodiments, the polypeptide that inhibits WISP1 is heterologous. As used herein, “heterologous” refers to a polypeptide which is not ordinarily produced by a host cell, e.g., the cell expressing the heterologous polypeptide, but rather, is derived from an organism different from the host cell. For example, the WISP1 inhibitor used herein is derived, for example, from a bacterial cell and expressed in, for example, a mammalian cell.

In some embodiments, the polypeptide that inhibits WISP1 is at least 50%, at least 60%, at least 70%, at least 80%, at least 90%, or 100% homologous to SEQ ID NOs 1-4 or 6. As used herein, the term “homology” or “homologous” as used herein is defined as the percentage of nucleotide or amino acid residues that are identical to the nucleotide or amino acid residues in the corresponding sequence on the target chromosome or polypeptide, after aligning the sequences and introducing gaps, if necessary, to achieve the maximum percent sequence identity. Alignment for purposes of determining percent nucleotide or amino acid sequence homology can be achieved in various ways that are within the skill in the art, for instance, using publicly available computer software such as BLAST, BLAST-2, ALIGN, ClustalW2 or Megalign (DNASTAR) software. Those skilled in the art can determine appropriate parameters for aligning sequences, including any algorithms needed to achieve maximal alignment over the full length of the sequences being compared. In some embodiments, a nucleic acid or amino acid sequence (e.g., DNA, RNA, or amino acid sequence), for example of, a WISP1-binding fragment or polypeptide, is considered “homologous” when the sequence is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to the corresponding native or unedited nucleic acid sequence (e.g., genomic sequence) or amino acid sequence of WISP1.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of ordinary skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. ligand-mediated receptor activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, a polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to an assay known in the art or described below herein. A functional fragment can comprise conservative substitutions of the sequences disclosed herein.

In some embodiments, a polypeptide described herein can be a variant of a polypeptide or molecule as described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity of the non-variant polypeptide. A wide variety of PCR-based site-specific mutagenesis approaches are known in the art and can be applied by the ordinarily skilled artisan.

A variant amino acid or DNA sequence can be at least 80%, at least 90%, at least 91%, at least 92%, at least 93%, at least 94%, at least 95%, at least 96%, at least 97%, at least 98%, at least 99%, identical to a native or reference sequence. The degree of homology (percent identity) between a native and a mutant sequence can be determined, for example, by comparing the two sequences using freely available computer programs commonly employed for this purpose on the world wide web (e.g., BLASTp or BLASTn with default settings).

Alterations of the native amino acid sequence can be accomplished by any of a number of techniques known in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of a polypeptide also can be substituted, generally with serine, to improve the oxidative stability of the molecule and prevent aberrant crosslinking. Conversely, cysteine bond(s) can be added to a polypeptide to improve its stability or facilitate oligomerization.

Antibodies that Inhibit WISP 1

In one aspect of any of the embodiments, described herein is a method treating or preventing a liver disease, the method comprising administering to a subject in need thereof an antibody or antibody reagent that inhibits WISP1 in a target cell.

In various embodiments, the agent described herein is an antibody or antigen-binding fragment thereof, or an antibody reagent that is specific for WISP1.

In another embodiment, the antibody or antibody reagent that inhibits WISP1 specifically binds to WISP1 polypeptide. In another embodiment, the antibody or antibody reagent, specifically binds to amino acid sequences SEQ ID NO: 1-4, or SEQ ID NO: 6.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as V_{H}), and a light (L) chain variable region (abbreviated herein as V_{L}). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies.

An antibody can have the structural features of IgA, IgG, IgE, IgD, or IgM (as well as subtypes and combinations thereof). Antibodies can be from any source, including mouse, rabbit, pig, sheep, goat, rat, and primate (human and non-human primate) and primatized antibodies. Antibodies also include midibodies, nanobodies, intrabodies, humanized antibodies, chimeric antibodies, and the like.

In one embodiment of any of the aspects, the antibody described herein is a humanized, monoclonal antibody or antigen-binding fragment thereof, or an antibody reagent. In another embodiment, the humanized antibody is a humanized monoclonal antibody. In another embodiment, the humanized antibody is a humanized polyclonal antibody. In yet another embodiment, the humanized antibody is for therapeutic use.

The anti-WISP1 antibody described herein can be a monospecific antibody or a monoclonal antibody. The term “monospecific antibody” refers to an antibody that displays a single binding specificity and affinity for a particular target, e.g., epitope. This term includes a “monoclonal antibody” or “monoclonal antibody composition,” which as used herein refer to a preparation of antibodies or fragments thereof of single molecular composition, irrespective of how the antibody was generated.

As used herein, the term “humanized antibody” refers to antibodies that comprise heavy and light chain variable domain sequences from a non-human species (e.g., a mouse, rat, sheep, or goat) but in which at least a portion of the V_{H} and/or V_{L} sequence has been altered to be more “human-like”, i.e., more similar to human germline variable sequences. Accordingly, “humanized” antibodies are a form of a chimeric antibody, that are engineered or designed to comprise minimal sequence derived from non-human immunoglobulin. For the most part, humanized antibodies are human immunoglobulins (recipient or acceptor antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fe), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). As used herein, a “composite human antibody” or “deimmunized antibody” are specific types of engineered or humanized antibodies designed to reduce or eliminate T cell epitopes from the variable domains.

A humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such humanized antibodies are chimeric antibodies (U.S. Pat. No. 4,816,567) where substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

In certain embodiments, the anti-WISP1 antibody is an intrabody. An intrabody is an intracellular antibody that functionally binds a target within a cell (See, generally, Hood et al., Immunology, Benjamin, N.Y., 2ND ed. (1984), Harlow and Lane, Antibodies. A Laboratory Manual, Cold Spring Harbor Laboratory (1988); Hunkapiller and Hood, Nature, 323, 15-16 (1986); and Rondon and Marasco, Annu Rev Microbiol, 51:257-83 (1997); U.S. Pat. Nos. 6,004,940; and 5,581,829; which are incorporated herein by reference in their entireties). Methods for intrabody production are well known to those of skill in the art, e.g. as described in WO 2002/086096. Antibodies will usually bind with at least a K_{D} of about 1 mM, more usually at least about 300 μM, typically at least about 10 μM, more typically at least about 30 μM, preferably at least about 10 μM, and more preferably at least about 3 μM or better.

In one embodiment, the anti-WISP1 antibody is a neutralizing antibody. In one embodiment, the anti-WISP1 antibody is a non-neutralizing antibody.

In some embodiments, the anti-WISP1 antibody is chimeric. As used herein, the term “chimeric”, as used in the context of an antibody, or sequence encoding an antibody refers to immunoglobin molecules characterized by two or more segments or portions derived from different animal species. For example, the variable region of the chimeric antibody is derived from a non-human mammalian antibody, such as murine monoclonal antibody, and the immunoglobin constant region is derived from a human immunoglobin molecule. The variable segments of chimeric antibodies are typically linked to at least a portion of an immunoglobulin constant region (Fe), typically that of a human immunoglobulin. Human constant region DNA sequences can be isolated in accordance with well-known procedures from a variety of human cells, such as immortalized B-cells (WO 87/02671; which is incorporated by reference herein in its entirety). The antibody can contain both light chain and heavy chain constant regions. The heavy chain constant region can include CH1, hinge, CH2, CH3, and, sometimes, CH4 regions. For therapeutic purposes, the CH2 domain can be deleted or omitted. Techniques developed for the production of “chimeric antibodies” are known in the art (see Morrison et al., Proc. Natl. Acad. Sci. 81:851-855 (1984); Neuberger et al., Nature 312:604-608 (1984); Takeda et al., Nature 314:452-454 (1985); which are incorporated by reference herein in their entireties), e.g., by splicing genes from a mouse, or other species, antibody molecule of appropriate antigen specificity together with genes from a human antibody molecule of appropriate biological activity.

In some embodiments of the compositions and methods described herein, the WISP1-binding domain comprises a variable light chain sequence, a variable heavy chain sequence, or both.

As understood by those of skill in the art, in a full-length antibody, each heavy chain is comprised of a heavy chain variable domain (abbreviated herein as HCVR or V_{H}) and a heavy chain constant region. The heavy chain constant region is comprised of three domains: C_{H}1, C_{H}2, and C_{H}3. Each light chain is comprised of a light chain variable domain (abbreviated herein LCVR as V_{L}) and a light chain constant region. The light chain constant region is comprised of one domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FRI, CDR1, FR2, CDR2, FR3, CDR3, FR4. This structure is well-known to those skilled in the art. The chains are usually linked to one another via disulfide bonds.

As used herein, the term “Complementarity Determining Regions” (“CDRs”), i.e., CDR1, CDR2, and CDR3) refers to the amino acid residues of a heavy or light chain variable domain the presence of which are necessary for specific antigen binding. Each variable domain typically has three CDR regions identified as CDR1, CDR2 and CDR3. Each complementarity determining region can comprise amino acid residues from a “complementarity determining region” as defined by Kabat (i.e., about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop. The term “CDR set” as used herein refers to a group of three CDRs that occur in a single heavy or light chain variable region capable of binding the antigen. The exact boundaries of these CDRs have been defined differently according to different systems. The system described by Kabat (Kabat et al, Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk, J. Mol. Biol, 196:901-917 (1987) and Chothia et al., Nature 342:877-883 (1989)) found that certain sub-portions within Kabat CDRs adopt nearly identical peptide backbone conformations, in spite of great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB). 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. CDRs can also be described as comprising amino acid residues from a “complementarity determining region” as defined by the IMGT, in some embodiments. The compositions and methods used herein may utilize CDRs defined according to any of these systems, although preferred embodiments use IMGT or Abysis defined CDRs. Nonetheless. The boundaries of the CDRs are clear in reference to either of these numbering conventions.

An immunoglobulin constant (C) domain refers to a heavy (C_{H}) or light (C_{L}) chain constant domain. Murine and human IgG heavy chain and light chain constant domain amino acid sequences are known in the art. With respect to the heavy chain, in some embodiments of the aspects described herein, the heavy chain of an antibody described herein can be an alpha (a), delta (A), epsilon (s), gamma (γ) or mu (p) heavy chain. In some embodiments of the aspects described herein, the heavy chain of an antibody described can comprise a human alpha (a), delta (A), epsilon (s), gamma (γ) or mu (p) heavy chain. Non-limiting examples of human constant region sequences have been described in the art, e.g., see U.S. Pat. No. 5,693,780 and Kabat E A et al., (1991) supra.

Accordingly, in embodiments of the compositions and methods described herein, the anti-WISP1 is comprised of a non-IgG framework.

As used herein, the terms “donor” and “donor antibody” refer to an antibody providing one or more CDRs. In an exemplary embodiment, the donor antibody is an antibody from a species different from the antibody from which the framework regions are obtained or derived. In some embodiments, the donor antibody is of a different isotype than the acceptor antibody. In the context of a humanized antibody, the term “donor antibody” refers to a non-human antibody providing one or more CDRs.

As used herein, the terms “acceptor” and “acceptor antibody” refer to the antibody providing or nucleic acid sequence encoding at least 80%, at least 85%, at least 90%, at least 95%, at least 98%, or 100% of the amino acid sequences of one or more of the framework regions. In some embodiments, the term “acceptor” refers to the antibody amino acid providing or nucleic acid sequence encoding the constant region(s). In yet another embodiment, the term “acceptor” refers to the antibody amino acid providing or nucleic acid sequence encoding one or more of the framework regions and the constant region(s). In a specific embodiment, the term “acceptor” refers to a human antibody amino acid or nucleic acid sequence that provides or encodes at least 80%, preferably, at least 85%, at least 90%, at least 95%, at least 98%, or 100% of the amino acid sequences of one or more of the framework regions. In accordance with this embodiment, an acceptor may contain at least 1, at least 2, at least 3, least 4, at least 5, or at least 10 amino acid residues that does (do) not occur at one or more specific positions of a human antibody. An acceptor framework region and/or acceptor constant region(s) may be, e.g., derived or obtained from a germline antibody gene, a mature antibody gene, a functional antibody (e.g., antibodies well known in the art, antibodies in development, or antibodies commercially available).

Human heavy chain and light chain acceptor sequences are known in the art. In some embodiments, the human heavy chain and light chain acceptor sequences are selected from the sequences listed from V-base (found on the worldwide web at vbase.mrc-cpe.cam.ac.uk/) or from IMGT™ the international IMMUNOGENETICS INFORMATION SYSTEM™ (found on the worldwide web at imgt.cines.fr/textes/IMGTrepertoire/LocusGenes/). In another embodiment of the technology disclosed herein, the human heavy chain and light chain acceptor sequences are selected from the sequences described in Table 3 and Table 4 of U.S. Patent Publication No. 2011/0280800, incorporated by reference herein in their entireties.

The compositions and methods described herein can, in some embodiments, comprise “antigen-binding fragments” or “antigen-binding portions” of an antibody. The term “antigen-binding fragment” or “WISP1-binding fragment” of an antibody refers to one or more fragments of an antibody that retain the ability to specifically bind to an antigen (e.g., WISP1).

Antigen-binding functions of an antibody can be performed by fragments of a full-length antibody. Such antibody fragment embodiments may also be incorporated in bispecific, dual specific, or multi-specific formats such as a dual variable domain (DVD-Ig) format; specifically binding to two or more different antigens. Non-limiting examples of antigen-binding fragments encompassed within the term “antigen-binding portion” of an antibody include (i) a Fab fragment, a monovalent fragment consisting of the V_{L}, V_{H}, C_{L}, and CHI domains; (ii) a F(ab′)2 fragment, a bivalent fragment comprising two Fab fragments linked by a disulfide bridge at the hinge region; (iii) a Fd fragment consisting of the V_{H} and CHI domains; (iv) a Fv fragment consisting of the V_{L} and V_{H} domains of a single arm of an antibody, (v) a dAb fragment (Ward et al. (1989) Nature, 341: 544-546; PCT Publication No. WO 90/05144), which comprises a single variable domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, for example, Bird et al. (1988) Science 242: 423-426; and Huston et al. (1988) Proc. Natl. Acad. Sci. USA 85: 5879-5883). Such single chain antibodies are also intended to be encompassed within the term “antigen-binding portion” of an antibody. Other forms of single chain antibodies, such as diabodies are also encompassed.

In some embodiments, the antibody reagent is a bispecific monoclonal antibody.

Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see, for example, Holliger et al. (1993) Proc. Natl. Acad. Sci. USA 90: 6444-6448; Poljak (1994) Structure 2: 1121-1123); Kontermann and Dubel eds., Antibody Engineering, Springer-Verlag, N.Y. (2001), p. 790 (ISBN 3-540-41354-5). In addition, single chain antibodies also include “linear antibodies” comprising a pair of tandem Fv segments (V_{H}-C_{H}1-V_{H}-C_{H}1) which, together with complementary light chain polypeptides, form a pair of antigen binding regions (Zapata et al. (1995) Protein Eng. 8(10): 1057-1062; and U.S. Pat. No. 5,641,870).

The term “Fe region” is used to define the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a C_{H}2 domain, and a C_{H}3 domain, and optionally comprises a C_{H}4 domain. Replacements of amino acid residues in the Fc portion to alter antibody effector function are known in the art (U.S. Pat. Nos. 5,648,260 and 5,624,821). The Fc portion of an antibody mediates several important effector functions, for example, cytokine induction, antibody-dependent cell cytotoxicity (ADCC), phagocytosis, complement dependent cytotoxicity (CDC), and half-life/clearance rate of antibody and antigen-antibody complexes. In some cases, these effector functions are desirable for therapeutic antibody but in other cases might be unnecessary or even deleterious, depending on the therapeutic objectives. Certain human IgG isotypes, particularly IgG1 and IgG3, mediate ADCC and CDC via binding to Fey receptors and complement C1q, respectively. Neonatal Fc receptors (FcRn) are the critical components determining the circulating half-life of antibodies. In still another embodiment, at least one amino acid residue is replaced in the constant region of the antibody, for example the Fc region of the antibody, such that effector functions of the antibody are altered.

The DNA sequences encoding the antibodies or antigen-binding fragments that specifically bind WISP1 as described herein. The nucleic acid sequences can also be modified, for example, by substituting the coding sequence for human heavy- and light-chain constant domains or framework regions in place of the homologous mammalian (e.g., murine) sequences (U.S. Pat. No. 4,816,567; Morrison, et al., Proc. Natl. Acad. Sci. USA, 81:6851 (1984)), or by covalently joining to the immunoglobulin coding sequence all or part of the coding sequence for a non-immunoglobulin polypeptide, as also described elsewhere herein.

Such non-immunoglobulin polypeptides can be substituted for the constant domains of an antibody, or they can be substituted for the variable domains of one antigen-binding site of an antibody to create a chimeric bivalent antibody comprising one antigen-binding site having specificity for one antigen of interest and another antigen-binding site having specificity for a different antigen of interest.

In some embodiments of the aspects described herein, the antibody or WISP1-binding fragment thereof comprises one, two, three, or four of the framework regions of a heavy chain variable region sequence which is at least 75%, 80%, 85%, 90%, 95% or 100% identical to one, two, three or four of the framework regions of the heavy chain variable region sequence from which it is derived. In some embodiments of the aspects described herein, the heavy chain variable framework region that is derived from said amino acid sequence consists of said amino acid sequence but for the presence of up to 10 amino acid substitutions, deletions, and/or insertions, preferably up to 10 amino acid substitutions. In some embodiments of the aspects described herein, the heavy chain variable framework region that is derived from said amino acid sequence consists of said amino acid sequence with 1, 2, 3, 4, 5, 6, 7, 8, 9 or 10 amino acid residues being substituted for an amino acid found in an analogous position in a corresponding non-human, primate, or human heavy chain variable framework region. In some embodiments of the aspects described herein, the antibody or antigen-binding fragment further comprises one, two, three or all four V_{H} framework regions derived from the V_{H} of a human or primate antibody. The primate or human heavy chain framework region of the antibody selected for use with the heavy chain CDR sequences described herein, can have, for example, at least 70% identity with a heavy chain framework region of the non-human parent antibody.

In some embodiments of the aspects described herein, the primate or human heavy chain framework region amino acid residues are from a natural primate or human antibody heavy chain framework region having at least 75% identity, at least 80% identity, at least 85% identity (or more) with the heavy chain framework regions of any of the antibodies described herein. In specific embodiments, the antibody or antigen-binding fragment further comprises one, two, three or all four V_{H} framework regions derived from a human heavy chain variable subfamily (e.g., one of subfamilies 1 to 7).

In some such embodiments of the aspects described herein, the antibody or WISP1-binding fragment thereof comprises one, two, three or four of the framework regions of a light chain variable region sequence which is at least 75%, 80%, 85%, 90%, 95%, or 100% identical to one, two, three or four of the framework regions of the light chain variable region sequence from which it is derived. In some embodiments of the aspects described herein, the light chain variable framework region that is derived from said amino acid sequence consists of said amino acid sequence but for the presence of up to 10 amino acid substitutions, deletions, and/or insertions, preferably up to 10 amino acid substitutions. In some embodiments of the aspects described herein, the light chain variable framework region that is derived from said amino acid sequence consists of said amino acid sequence with 1, 2, 3, 4, 5, 6, 7, 8, 9 or 10 amino acid residues being substituted for an amino acid found in an analogous position in a corresponding non-human, primate, or human light chain variable framework region. In some embodiments of the aspects described herein, the antibody or antigen-binding fragment further comprises one, two, three or all four V_{L} framework regions derived from the V_{L} of a human or primate antibody. The primate or human light chain framework region of the antibody selected for use with the light chain CDR sequences described herein, can have, for example, at least 70% identity with a light chain framework region of the non-human parent antibody.

In some embodiments of the aspects described herein, the primate or human light chain framework region amino acid residues are from a natural primate or human antibody light chain framework region having at least 75% identity, at least 80% identity, at least 85% identity (or more) with the light chain framework regions of any of the antibodies described herein. In some embodiments, the antibody or antigen-binding fragment further comprises one, two, three or all four V_{L} framework regions derived from a human light chain variable kappa subfamily. In some embodiments, the antibody or antigen-binding fragment further comprises one, two, three or all four V_{L} framework regions derived from a human light chain variable lambda subfamily.

In some embodiments of the aspects described herein, the position of one or more CDRs along the V_{H} (e.g., CDR1, CDR2, or CDR3) and/or V_{L} (e.g., CDR1, CDR2, or CDR3) region of an antibody described herein can vary, i.e., be shorter or longer, by one, two, three, four, five, or six amino acid positions so long as immunospecific binding to the antigen of interest is maintained (e.g., substantially maintained, for example, at least 50%, at least 60%, at least 70%, at least 80%, at least 90%, at least 95% of the binding of the original antibody from which it is derived). For example, in some embodiments, the position defining a CDR can vary, i.e., be shorter or longer, by shifting the N-terminal and/or C-terminal boundary of the CDR by one, two, three, four, five, or six amino acids, relative to the CDR position of any one of the antibodies described herein, so long as immunospecific binding to the antigen of interest is maintained (e.g., substantially maintained, for example, at least 50%, at least 60%, at least 70%, at least 80%, at least 90%, at least 95% of the binding of the original antibody from which it is derived). In other embodiments, the length of one or more CDRs along the V_{H} (e.g., CDR1, CDR2, or CDR3) and/or V_{L} (e.g., CDR1, CDR2, or CDR3) region of an antibody described herein can vary (e.g., be shorter or longer) by one, two, three, four, five, or more amino acids, so long as immunospecific binding to the antigen of interest is maintained (e.g., substantially maintained, for example, at least 50%, at least 60%, at least 70%, at least 80%, at least 90%, at least 95% of the binding of the original antibody from which it is derived).

In some embodiments of the aspects described herein, one, two or more mutations (e.g., amino acid substitutions) are introduced into the Fc region of any of the antibodies described herein or a fragment thereof (e.g., C_{H}2 domain (residues 231-340 of human IgG1) and/or C_{H}3 domain (residues 341-447 of human IgG1) and/or the hinge region, with numbering according to the Kabat numbering system (e.g., the EU index in Kabat)) to alter one or more functional properties of the antibody, such as serum half-life, complement fixation, Fc receptor binding and/or antigen-dependent cellular cytotoxicity.

In some embodiments of the aspects described herein, one, two or more mutations (e.g., amino acid substitutions) are introduced into the hinge region of the Fc region (CHI domain) such that the number of cysteine residues in the hinge region are altered (e.g., increased or decreased) as described in, e.g., U.S. Pat. No. 5,677,425. The number of cysteine residues in the hinge region of the C_{H}I domain can be altered to, e.g., facilitate assembly of the light and heavy chains, or to alter (e.g., increase or decrease) the stability of the antibody.

In some embodiments of the aspects described herein, one, two or more mutations (e.g., amino acid substitutions) are introduced into the Fc region of an antibody described herein or an antigen-binding fragment thereof (e.g., C_{H}2 domain (residues 231-340 of human IgG1) and/or C_{H}3 domain (residues 341-447 of human IgG1) and/or the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody or fragment thereof that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

The term “CDR-grafted antibody” refers to antibodies which comprise heavy and light chain variable region sequences from one species, but in which the sequences of one or more of the CDR regions of V_{H} and/or V_{L} are replaced with CDR sequences of another species, such as antibodies having human heavy and light chain variable regions in which one or more of the human CDRs (e.g., CDR3) has been replaced with mouse CDR sequences. CDR-grafted antibodies described herein comprise heavy and light chain variable region sequences from a human antibody wherein one or more of the CDR regions of V_{H} and/or V_{L} are replaced with CDR sequences of the non-human antibodies described herein.

The anti-WISP1 antibody described herein can be engineered for improved binding specificity or pharmacokinetic properties for therapeutic use. Specificity of binding can be assayed, for example, by competition assays using the antigen (e.g., WISP1 or a polypeptide fragment thereof), in comparison to competition with one or more unrelated or different antigens. A variety of immunoassay formats are appropriate for selecting agents, antibodies, or other ligands that specifically bind WISP1. Specific binding can be influenced by, for example, the affinity and avidity of the agent described herein (e.g., a polypeptide or anti-WISP1 antibody) and the concentration of agent. The person of ordinary skill in the art can determine appropriate conditions under which the agents described herein selectively bind the WISP1 using any suitable methods, such as titration of an agent in a suitable binding assay.

As used herein, the term “key” residues refers to certain residues within the variable domain that have more impact on the binding specificity and/or affinity of an antibody, in particular a humanized antibody, than others. A key residue includes, but is not limited to, one or more of the following: a residue that is adjacent to a CDR, a potential glycosylation site (can be either N- or O-glycosylation site), a rare residue, a residue capable of interacting with the antigen, a residue capable of interacting with a CDR, a canonical residue, a contact residue between heavy chain variable domain and light chain variable domain, a residue within the Vernier zone, and a residue in the region that overlaps between the Chothia definition of a variable heavy chain CDR/and the Kabat definition of the first heavy chain framework.

The anti-WISP1 antibodies described herein can be engineered antibodies. As used herein, the term “engineered” refers to the aspect of having been manipulated by the hand of man. For example, a locus is considered to be “engineered” when two or more sequences, that are not linked together in that order in nature in that locus, are manipulated by the hand of man to be directly linked to one another in the engineered locus. For example, in some embodiments of the present invention, an engineered locus comprises various Ig sequences with a non-native V segment, all of which are found in nature, but are not found in the same locus or are not found in that order in the locus in nature. As is common practice and is understood by those in the art, progeny and copies of an engineered polynucleotide (and/or cells or animals comprising such polynucleotides) are typically still referred to as “engineered” even though the actual manipulation was performed on a prior entity.

A WISP1-binding polypeptide, antibody, antibody reagent, or antigen-binding portion thereof, can be part of a larger immunoadhesion molecule or composition of molecules, formed by covalent or noncovalent association of the antibody antigen-binding portion with one or more other proteins or peptides. Examples of such immunoadhesion molecules include use of the streptavidin core region to make a tetrameric scFv molecule (Kipriyanov et al. (1995) Human Antibod. Hybridomas 6:93-101) and use of a cysteine residue, a marker peptide and a C-terminal polyhistidine tag to make bivalent and biotinylated scFv molecules (Kipriyanov et al. (1994) Mol. Immunol. 31:1047-1058). Antibody portions, such as Fab and F(ab′).sub.2 fragments, can be prepared from whole antibodies using conventional techniques, such as papain or pepsin digestion, respectively, of whole antibodies. Moreover, antibodies, antigen-binding portions thereof, and immunoadhesion molecules can be obtained using standard recombinant DNA techniques. A target binding protein, such as an antigen-binding portion of an antibody may also be part of a dual variable domain (DVD-Ig).

Antibodies and antibody reagents that are therapeutic and/or specific for any particular target antigen (e.g., WISP1) are readily selected by one of skill in the art from known antibodies or antibody reagents, e.g. from FDA-approved therapeutic antibody reagents and/or commercially available antibody reagents which are listed in catalogs according to their target specificity.

In another embodiment, the anti-WISP1 antibody is any known anti-WISP1 antibodies in the art, or any anti-WISP1 antibodies that are yet to be discovered. Exemplary anti-WISP1 antibodies known in the art include, but are not limited to, anti-WISP1 antibodies sold by Abeam (e.g., ab60114; ab65943), RND Systems (e.g., mab1680), and Sigma-Aldrich (e.g., SAB2501114).

In another embodiment, the anti-WISP1 antibody is selected from the Table 1 (Table of Antibodies) below. In another embodiment, the anti-WISP1 antibody comprises any one of the amino acid sequences of SEQ ID NOs: 12-120.

TABLE 1
Table of Antibodies (Anti-WISP1)

| | Manufacturer | | CDR | |
|---|---|---|---|---|
| Antibody Name | (Catalog #) | Source | Sequences | References |
| Mouse WISP- | R & D Systems | Monoclonal | SEQ ID NOs: | 1. Pennica, D. et al. (1998) Proc. Natl. |
| 1/CCN4 | (MAB1680) | Rat IgG2B | 12-51 | Acad. Sci. USA 95: 14717. |
| Antibody | | Clone # 214203 | | 2. Tanada, S. et al. (2001) Oncogene |
| mab1680 | | | | 20: 5525. |
| | | | | 3. Brigstock, D. R. et al. (2003) J. Clin. |
| | | | | Pathol. Mol. Pathol. 56: 127. |
| | | | | 4. Desnoyers, L. (2004) Curr. Pharm. |
| | | | | Des. 10: 3913. |
| | | | | 5. Brigstock, D. R. (2003) J. Endocrinol. |
| | | | | 178: 169. |
| | | | | 6. Hashimoto, Y. et al. (1998) J. Exp. |
| | | | | Med. 187: 289. |
| | | | | 7. SwissProt Accession # O54775. |
| | | | | 8. SwissProt Accession # Q99PP0. |
| | | | | 9. SwissProt Accession # O95388 |
| | | | | 10. French, D. M. et al. (2004) Am. J. |
| | | | | Pathol. 165: 855. |
| | | | | 11. Parisi, M. S. et al. (2006) Bone |
| | | | | 38: 671. |
| Human WISP- | R & D Systems | Human Mouse | | 1. Pennica, D. et al. (1998) Proc. Natl. |
| 1/CCN4 | (MAB1627) | IgG2A Clone # | | Acad. Sci. USA 95: 14717. |
| Antibody | | 213611 | | 2. Tanada, S. et al. (2001) Oncogene |
| mab1680 | | | | 20: 5525. |
| | | | | 3. Brigstock, D. R. et al. (2003) J. Clin. |
| | | | | Pathol. Mol. Pathol. 56: 127. |
| | | | | 4. Desnoyers, L. (2004) Curr. Pharm. |
| | | | | Des. 10: 3913. |
| | | | | 5. Brigstock, D. R. (2003) J. Endocrinol. |
| | | | | 178: 169. |
| | | | | 6. Li, Z. (2005) GenBank Accession # |
| | | | | AAP43925. |
| | | | | 7. Li, Z. (2005) GenBank Accession # |
| | | | | AAP43926. |
| | | | | 8. Li, Z. (2005) GenBank Accession # |
| | | | | AAP43924. |
| | | | | 9. Cervello, M. et al. (2004) Ann. N.Y. |
| | | | | Acad. Sci. 1028: 432. |
| | | | | 10. French, D. M. et al. (2004) Am. J. |
| | | | | Pathol. 165: 855. |
| | | | | 11. Parisi, M. S. et al. (2006) Bone |
| | | | | 38: 671. |
| WISP-1/CCN4 | Novus Biologicals | Sheep | SEQ ID NOs: | 1. Quiros et al. J Clin Inv. (2017)) |
| Antibody | (AF1680) | | 52-90 | 2. Colston et al. Am. J. Physiol. Heart |
| | | | | Circ. (2007). |
| Anti-WISP1 | Sigma Aldrich | Goat | SEQ ID NOs: | |
| antibody | (SAB2501114) | | 91-110 | |
| produced in | | | | |
| goat | | | | |
| Anti-WISP1 | Abcam | Rabbit | SEQ ID NOs: | |
| antibody | (ab60114) | | 111-120 | |
| Anti-WISP1 | Abcam | Goat | SEQ ID NOs: | |
| antibody | (ab65943) | | 91-110 | |

*Sequences were obtained from AbYsis available on the world-wide web at <abysis.org>.

In another embodiment, the anti-WISP1 antibody is a humanized anti-WISP1 antibody derived from any known, or yet to be discovered, non-human anti-WISP1 antibody.

In one embodiment, the antibody or antibody reagent binds to an amino acid sequence that corresponds to the amino acid sequence encoding WISP1 (SEQ ID NO: 1).

In another embodiment, the anti-WISP1 antibody or antibody reagent binds to an amino acid sequence that comprises the sequence of SEQ ID NO: 1; or binds to an amino acid sequence that comprises a sequence with at least 70%, at least 80%, at least 85%, at least 90%, at least 95%, at least 96%, at least 97%, at least 98%, at least 99% or greater sequence identity to the sequence of SEQ ID NO: 1. In one embodiment, the anti-WISP1 antibody or antibody reagent binds to an amino acid sequence that comprises the entire sequence of SEQ ID NO: 1. In another embodiment, the antibody or antibody reagent binds to an amino acid sequence that comprises a fragment of the sequence of SEQ ID NO: 1, wherein the fragment is sufficient to bind its target, e.g., WISP1, and result in the inhibition of WISP1 level and/or activity.

In certain embodiments, the antibody or antibody reagent binds to an amino acid sequence that corresponds to the amino acid sequence encoding various human WISP1 isoforms (SEQ ID NOs: 2, 3, or 4).

In another embodiment, the anti-WISP1 antibody or antibody reagent binds to an amino acid sequence that comprises the sequence of SEQ ID NOs: 2, 3, or 4; or binds to an amino acid sequence that comprises a sequence with at least 70%, at least 80%, at least 85%, at least 90%, at least 95%, at least 96%, at least 97%, at least 98%, at least 99% or greater sequence identity to the sequence of SEQ ID NOs: 2, 3, or 4. In one embodiment, the anti-WISP1 antibody or antibody reagent binds to an amino acid sequence that comprises the entire sequence of SEQ ID NOs: 2, 3, or 4. In another embodiment, the antibody or antibody reagent binds to an amino acid sequence that comprises a fragment of the sequence of SEQ ID NOs: 2, 3, or 4, wherein the fragment is sufficient to bind its target, e.g., WISP1, and result in the inhibition of WISP1 level and/or activity.

In another embodiment, the antibody or antibody reagent binds to an amino acid sequence that corresponds to the amino acid sequence encoding mouse WISP1 (SEQ ID NO: 6).

In another embodiment, the antibody or antibody reagent binds to an amino acid sequence that comprises the sequence of SEQ ID NO: 6; or binds to an amino acid sequence that comprises a sequence with at least 70%, at least 80%, at least 85%, at least 90%, at least 95%, at least 96%, at least 97%, at least 98%, at least 99% or greater sequence identity to the sequence of SEQ ID NO: 6. In one embodiment, the anti-WISP1 antibody or antibody reagent binds to an amino acid sequence that comprises the entire sequence of SEQ ID NO: 6. In another embodiment, the antibody or antibody reagent binds to an amino acid sequence that comprises a fragment of the sequence of SEQ ID NO: 6, wherein the fragment is sufficient to bind its target, e.g., WISP1, and result in the inhibition of WISP1 level and/or activity.

Accordingly, in some embodiments, described herein are humanized antibodies comprising one or more variable domains comprising one or more CDRs encoded by the variable heavy chain and light chain sequences of SEQ ID NOs: 12-120.

In another embodiment, the antibody or antibody reagent comprises an amino acid sequence with at least 70% homology to any one of SEQ ID NOs: 12-120. In another embodiment, the antibody or antibody reagent CDR comprises an amino acid sequence with at least 70% homology to any one of SEQ ID NOs: 12-120. In another embodiment, the antibody or antibody reagent comprises an amino acid sequence with at least 90% homology to any one of SEQ ID NOs: 12-120.

Stated another way, in some embodiments, the antibody or antibody reagent that inhibits WISP1 is at least 50%, at least 60%, at least 70%, at least 80%, at least 90%, or 100% homologous to SEQ ID NOs 1-4, 6, or SEQ ID NOs: 12-120.

In other embodiments, a nucleic acid or amino acid sequence (e.g., DNA, RNA, or amino acid sequence), for example of, a WISP1-binding fragment or anti-WISP1 antibody, is considered “homologous” when the sequence is at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to the corresponding native or unedited nucleic acid sequence (e.g., genomic sequence) or amino acid sequence of an antibody that specifically binds to WISP1.

One aspect provided herein is a composition comprising any of the anti-WISP1 antibodies or antibody reagents described herein. In one embodiment, the composition further comprises a pharmaceutically acceptable carrier. In one embodiment, the composition is a pharmaceutical composition.

Nucleic Acids that Inhibit WISP1

In one embodiment, the agent that inhibits WISP1 is an antisense oligonucleotide. As used herein, an “antisense oligonucleotide” refers to a synthesized nucleic acid sequence that is complementary to a DNA or mRNA sequence, such as that of a microRNA. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under cellular conditions to a gene, e.g., WISP1. Thus, oligonucleotides are chosen that are sufficiently complementary to the target, i.e., that hybridize sufficiently well and with sufficient specificity in the context of the cellular environment, to give the desired effect. For example, an antisense oligonucleotide that inhibits WISP1 may comprise at least 5, at least 10, at least 15, at least 20, at least 25, at least 30, or more bases complementary to a portion of the coding sequence of the human WISP1 gene (e.g., SEQ ID NO: 5) or the mouse WISP1 gene (e.g., SEQ ID NO: 7).

In one embodiment, WISP1 is depleted from the cell's genome using any genome editing system including, but not limited to, zinc finger nucleases, TALENS, meganucleases, and CRISPR/Cas systems. In one embodiment, the genomic editing system used to incorporate the nucleic acid encoding one or more guide RNAs into the cell's genome is not a CRISPR/Cas system; this can prevent undesirable cell death in cells that retain a small amount of Cas enzyme/protein. It is also contemplated herein that either the Cas enzyme or the sgRNAs are each expressed under the control of a different inducible promoter, thereby allowing temporal expression of each to prevent such interference.

When a nucleic acid encoding one or more sgRNAs and a nucleic acid encoding an RNA-guided endonuclease each need to be administered in vivo, the use of an adenovirus associated vector (AAV) is specifically contemplated. Other vectors for simultaneously delivering nucleic acids to both components of the genome editing/fragmentation system (e.g., sgRNAs, RNA-guided endonuclease) include lentiviral vectors, such as Epstein Barr, Human immunodeficiency virus (HIV), and hepatitis B virus (HBV). Each of the components of the RNA-guided genome editing system (e.g., sgRNA and endonuclease) can be delivered in a separate vector as known in the art or as described herein.

In one embodiment, the agent inhibits WISP1 by RNA inhibition. Inhibitors of the expression of a given gene can be an inhibitory nucleic acid. In some embodiments of any of the aspects, the inhibitory nucleic acid is an inhibitory RNA (iRNA). The RNAi can be single stranded or double stranded.

The iRNA can be siRNA, shRNA, endogenous microRNA (miRNA), or artificial miRNA. In one embodiment, an iRNA as described herein effects inhibition of the expression and/or activity of a target, e.g. WISP1. In some embodiments of any of the aspects, the agent is siRNA that inhibits WISP1. In some embodiments of any of the aspects, the agent is shRNA that inhibits WISP1.

One skilled in the art would be able to design siRNA, shRNA, or miRNA to target WISP1, e.g., using publically available design tools. siRNA, shRNA, or miRNA is commonly made using companies such as Dharmacon (Layfayette, CO) or Sigma Aldrich (St. Louis, MO).

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions

The RNA of an iRNA can be chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference.

In one embodiment, the agent is miRNA that inhibits WISP1. microRNAs are small non-coding RNAs with an average length of 22 nucleotides. These molecules act by binding to complementary sequences within mRNA molecules, usually in the 3′ untranslated (3′UTR) region, thereby promoting target mRNA degradation or inhibited mRNA translation. The interaction between microRNA and mRNAs is mediated by what is known as the “seed sequence”, a 6-8-nucleotide region of the microRNA that directs sequence-specific binding to the mRNA through imperfect Watson-Crick base pairing. More than 900 microRNAs are known to be expressed in mammals. Many of these can be grouped into families on the basis of their seed sequence, thereby identifying a “cluster” of similar microRNAs. A miRNA can be expressed in a cell, e.g., as naked DNA. A miRNA can be encoded by a nucleic acid that is expressed in the cell, e.g., as naked DNA or can be encoded by a nucleic acid that is contained within a vector.

In one embodiment, the agent that downmodulates WISP1 is miRNA-15a. miRNA-15a is a non-coding RNA that regulates gene expression in a number of organs including the liver. miRNA-15a sequences are known for a number of species, e.g., human miRNA-15a, e.g., miRBase Accession number M10000069, and mouse miRNA-15a, e.g., miRBase Accession number M10000564. Human miRNA-15a comprises the sequence of SEQ ID NO: 8. miRNA-15a can refer to human miRNA-15a, including naturally occurring variants, molecules, and alleles thereof. For example, miRNA-15a can be mouse miRNA-15a, having the sequence of SEQ ID NO: 10.

In one embodiment, the agent, e.g., the miRNA-15A has a sequence corresponding to the sequence of SEQ ID NO: 8; or comprises the sequence of SEQ ID NO: 8; or comprises a sequence with at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 8.

In one embodiment, the agent, e.g., the miRNA-15a has a sequence corresponding to the sequence of SEQ ID NO: 10; or comprises the sequence of SEQ ID NO: 10; or comprises a sequence with at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 10.

In one embodiment, the agent that downmodulates WISP1 is miRNA-412. miRNA-412 sequences are known for a number of species, e.g., human miRNA-412, e.g., miRBase Accession number MI0001464, and mouse miRNA-412, e.g., miRBase Accession number MI0001164. Human miRNA-412 comprises the sequence of SEQ ID NO: 9. miRNA-412 can refer to human miRNA-15a, including naturally occurring variants, molecules, and alleles thereof. For example, miRNA-15a can be mouse miRNA-412, having the sequence of SEQ ID NO: 11.

In one embodiment, the agent, e.g., the miRNA-412 has a sequence corresponding to the sequence of SEQ ID NO: 9; or comprises the sequence of SEQ ID NO: 9; or comprises a sequence with at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 9.

In one embodiment, the agent, e.g., the miRNA-412, has a sequence corresponding to the sequence of SEQ ID NO: 11; or comprises the sequence of SEQ ID NO: 11; or comprises a sequence with at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 11.

The agent may result in gene silencing of the target gene (e.g., WISP1), such as with an RNAi molecule (e.g. siRNA or miRNA). This entails a decrease in the mRNA level in a cell for a target by at least about 5%, about 10%, about 20%, about 30%, about 40%, about 50%, about 60%, about 70%, about 80%, about 90%, about 95%, about 99%, about 100% of the mRNA level found in the cell without the presence of the agent. In one preferred embodiment, the mRNA levels are decreased by at least about 70%, about 80%, about 90%, about 95%, about 99%, about 100%. One skilled in the art will be able to readily assess whether the siRNA, shRNA, or miRNA effective target e.g., WISP1, for its downregulation, for example by transfecting the siRNA, shRNA, or miRNA into cells and detecting the levels of a gene (e.g., WISP1) found within the cell via western-blotting or PCR-based assays.

The agent may be contained in and thus further include a vector. Many such vectors useful for transferring exogenous genes into target mammalian cells are available. The vectors may be episomal, e.g. plasmids, virus-derived vectors such cytomegalovirus, adenovirus, etc., or may be integrated into the target cell genome, through homologous recombination or random integration, e.g. retrovirus-derived vectors such as MMLV, HIV-1, ALV, etc. In some embodiments, combinations of retroviruses and an appropriate packaging cell line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide (e.g., a WISP1 inhibitor) from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector.

One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector.

Another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages).

Another example of a non-integrative vector is a minicircle vector. Minicircle vectors are circularized vectors in which the plasmid backbone has been released leaving only the eukaryotic promoter and cDNA(s) that are to be expressed.

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

### Engineered Hepatic Stellate Cells (HSCs)

In one aspect, described herein is a method of generating a hepatic stellate cell, or population thereof, that expresses an agent that inhibits WISP1, comprising contacting the cell with any of the agents that inhibit WISP1 described herein, and culturing the cell for a sufficient time to allow for expression of the agent.

In one embodiment, the cell is a quiescent cell. Methods for identifying a quiescent cell are described herein above.

In one embodiment, the contacting comprises contacting the cell with an agent or a vector that encodes the agent. For example, contacting can comprise, but is not limited to, transduction, nucleofection, electroporation, direct injection (e.g., into the HSC), and/or transfection. One skilled in the art can contact the cell with an agent described herein using techniques known in the art.

In one embodiment, the agent is a miRNA. In one embodiment, the miRNA is miRNA-15a or miRNA412.

In one embodiment, the cell is cultured in Dulbecco's Modified Eagle Medium (DMEM) supplemented with 20% (weight/volume) fetal bovine serum (FBS) and 1% (weight/volume) penicillin/streptomycin under standard culturing conditions.

In one embodiment, the cell is cultured for at least 1 hour to allow for expression of the agent. In another embodiment, the cell is cultured for at least 2, 3, 4, 5, 6, 7, 8, 9, 10, 11, 12, 13, 14, 15, 16, 17, 18, 19, 20, 21, 22, 23, 24, 36, 48, 60, 72, 96, 120, 144, or more hours to all for expression of the agent. One skilled in the art can determine if an agent is expressed in the cell following culturing using standard techniques in the art, for example, PCR-based assay to detect miRNA expression.

In one embodiment, the cell transiently expresses the agent. In another embodiment, the expression of the agent in integrated into the genome of the cell, e.g., such that the cell's progeny expresses the agent.

One aspect provides a cell line comprising HSCs expressing an agent that inhibits WISP1 generated using methods described herein. HSCs that express an agent that inhibits WISP1 can be in a pharmaceutically acceptable carrier, e.g., for administration to a subject in need of treatment, e.g., for liver disease.

Another aspect provides a pharmaceutical composition comprising a population of HSCs expressing an agent that inhibits WISP1 generated using methods described herein and a pharmaceutically acceptable carrier.

### Compositions and Pharmaceutical Compositions

In one aspect, described herein is a composition comprising any of any of the agents described herein. In one aspect, described herein is a pharmaceutical composition comprising any of any of the agents described herein.

In another aspect, described herein is a composition comprising an antibody or antibody reagent that inhibits WISP1. In another aspect, described herein is a pharmaceutical composition comprising an antibody or antibody reagent that inhibits WISP1.

In one embodiment, a composition or pharmaceutical composition described herein can comprise at least 2, 3, 4, 5, or more agents described herein. For example, a composition can comprise an siRNA that inhibits WISP1 and an anti-WISP1 antibody reagent. Alternatively, the composition can comprise two anti-WISP1 antibody-reagents.

In one embodiment of any of the aspects, the composition is formulated for the treatment or prevention of liver disease. For the clinical use of the methods described herein, administration of agents that inhibit WISP1 (e.g., antibodies, antibody reagents, or WISP1-binding fragments thereof) described herein can include formulation into pharmaceutical compositions or pharmaceutical formulations for parenteral administration, e.g., intravenous; mucosal, e.g., intranasal; ocular, or other mode of administration. In some embodiments, the agents described herein can be administered along with any pharmaceutically acceptable carrier compound, material, or composition which results in an effective treatment in the subject. Thus, a pharmaceutical formulation for use in the methods described herein can contain an antibody or antigen-binding fragment thereof as described herein in combination with one or more pharmaceutically acceptable ingredients.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, tale magnesium, calcium or zinc stearate, or steric acid), or solvent encapsulating material, involved in maintaining the stability, solubility, or activity of, an antibody or antigen-binding fragment thereof. Each carrier must be “acceptable” in the sense of being compatible with the other ingredients of the formulation and not injurious to the patient. The terms “excipient”, “carrier”, “pharmaceutically acceptable carrier” or the like are used interchangeably herein.

Therapeutic formulations of the agents or inhibitors of WISP1 described herein can be prepared for storage by mixing the antibodies or antigen-binding fragments having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Exemplary lyophilized antibody formulations are described in WO 97/04801, expressly incorporated herein by reference.

Optionally, but preferably, the formulations comprising the compositions described herein contain a pharmaceutically acceptable salt, typically, e.g., sodium chloride, and preferably at about physiological concentrations. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are examples of preservatives. Optionally, the formulations of the invention can include a pharmaceutically acceptable surfactant at a concentration of 0.005 to 0.02%.

The therapeutic formulations of the compositions comprising agents (e.g., antibodies, antibody reagents, and WISP1-binding fragments thereof) described herein can also contain more than one active compound as necessary for the particular indication being treated, preferably those with complementary activities that do not adversely affect each other. Alternatively, the composition can comprise a cytotoxic agent, cytokine, or growth inhibitory agent, for example. Such molecules are suitably present in combination in amounts that are effective for the purpose intended.

The active ingredients of the therapeutic formulations of the compositions comprising agents described herein can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

In some embodiments, the pharmaceutical composition further comprises a lipid vehicle. Exemplary lipid vehicles include, but are not limited to, liposomes, micelles, exosomes, lipid emulsions, and lipid-drug complex.

In some embodiments, the pharmaceutical composition further comprises a particle or polymer-based vehicle. Exemplary particle or polymer-based vehicles include, but are not limited to, nanoparticles, microparticles, polymer microspheres, or polymer-drug conjugates.

In some embodiments, sustained-release preparations can be used. Suitable examples of sustained-release preparations include semipermeable matrices of solid hydrophobic polymers containing the antibodies or antigen-binding fragments in which the matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they can denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization can be achieved by modifying sulfhydryl residues, lyophilizing from acidic solutions, controlling moisture content, using appropriate additives, and developing specific polymer matrix compositions.

The therapeutic formulations to be used for in vivo administration, such as parenteral administration, in the methods described herein can be sterile, which is readily accomplished by filtration through sterile filtration membranes, or other methods known to those of skill in the art.

### Administration

In some embodiments, the methods described herein relate to treating a subject having or diagnosed as having a liver disease comprising administering an agent that inhibits WISP1 as described herein. Subjects having a liver disease can be identified by a physician using current methods of diagnosing a condition. Symptoms and/or complications of a liver disease, which characterize this disease and aid in diagnosis are well known in the art and include but are not limited to, fatigue, weight loss, pain, yellowing of skin and/or eyes, and dark urine. Tests that may aid in a diagnosis of, e.g. a liver disease, include but are not limited example blood tests, non-invasive imaging, and/or tissue biopsy. A family history of a liver disease will also aid in determining if a subject is likely to have the condition or in making a diagnosis of a liver disease.

The agents and compositions described herein (e.g., that inhibit WISP1) can be administered to a subject having or diagnosed as having a liver disease. In some embodiments, the methods described herein comprise administering an effective amount of an agent to a subject in order to alleviate at least one symptom of the liver disease. As used herein, “alleviating at least one symptom of the liver disease” is ameliorating any condition or symptom associated with the liver disease (e.g., fatigue, weight loss, pain, yellowing of skin and/or eyes, or dark urine). As compared with an equivalent untreated control, such reduction is by at least 5%, 10%, 20%, 40%, 50%, 60%, 80%, 90%, 95%, 99% or more as measured by any standard technique. A variety of means for administering the agents and compositions described herein to subjects are known to those of skill in the art. In one embodiment, the agent is administered systemically or locally (e.g., to the liver). In one embodiment, the agent is administered intravenously. In one embodiment, the agent is administered continuously, in intervals, or sporadically. The route of administration of the agent will be optimized for the type of agent being delivered (e.g., a miRNA, a cell, or an RNAi), and can be determined by a skilled practitioner.

The agents and pharmaceutical compositions described herein can be administered to a subject in need thereof by any appropriate route which results in an effective treatment in the subject. As used herein, the terms “administering,” and “introducing” are used interchangeably and refer to the placement of an agent or pharmaceutical composition described herein (e.g., an antibody, antibody reagent, or WISP1-binding fragment thereof) into a subject by a method or route which results in at least partial localization of such agents at a desired site, such as a site of infection or cancer, such that a desired effect(s) is produced. An agent or pharmaceutical composition can be administered to a subject by any mode of administration that delivers the agent systemically or to a desired surface or target, and can include, but is not limited to, injection, infusion, instillation, and inhalation administration. To the extent that various agents can be protected from inactivation in the gut, oral administration forms are also contemplated. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

The agents (e.g., antibodies, antibody reagents, and WISP1-binding fragments thereof), are formulated, dosed, and administered in a fashion consistent with good medical practice. Factors for consideration in this context include the particular disorder being treated, the particular subject being treated, the clinical condition of the individual subject, the cause of the disorder, the site of delivery of the agent, the method of administration, the scheduling of administration, and other factors known to medical practitioners. The “therapeutically effective amount” of the agents to be administered are governed by such considerations, and refers to the minimum amount necessary to ameliorate, treat, or stabilize, the cancer; to increase the time until progression (duration of progression free survival) or to treat or prevent the occurrence or recurrence of a liver disease. The agents are optionally formulated, in some embodiments, with one or more additional therapeutic agents currently used to prevent or treat the infection, for example. The effective amount of such other agents depends on the amount of agent (e.g., antibodies and WISP1-binding fragments thereof) present in the formulation, the type of disorder or treatment, and other factors discussed above. These are generally used in the same dosages and with administration routes as used herein before or about from 1 to 99% of the heretofore employed dosages.

### Dosage

For the treatment of liver diseases, as described herein, the appropriate dosage of an agent (e.g., an antibody, antibody reagent, or WISP1-binding fragment thereof) will depend on the type of liver disease to be treated, as defined above, the severity and course of the disease, whether the agent is administered for preventive or therapeutic purposes, previous therapeutic indications, the subject's clinical history and response to the agent, and the discretion of the attending physician. The agent is suitably administered to the subject at one time or over a series of treatments. In a combination therapy regimen, the agent and the one or more additional therapeutic agents described herein are administered in a therapeutically effective or synergistic amount.

As used herein, a “unit dosage form” refers to a dosage for suitable one administration. By way of example a unit dosage form can be an amount of therapeutic disposed in a delivery device, e.g., a syringe or intravenous drip bag. In one embodiment, a unit dosage form is administered in a single administration. In another, embodiment more than one unit dosage form can be administered simultaneously.

The dosage of the agent as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

The dosage ranges for the therapeutic agents depend upon the potency, and encompass amounts large enough to produce the desired effect. The dosage should not be so large as to cause unacceptable adverse side effects. Generally, the dosage will vary with the age, condition, and sex of the patient and can be determined by one of skill in the art. The dosage can also be adjusted by the individual physician in the event of any complication. In some embodiments, the dosage ranges from 0.001 mg/kg body weight to 100 mg/kg body weight. In some embodiments, the dose range is from 5 g/kg body weight to 100 g/kg body weight. Alternatively, the dose range can be titrated to maintain serum levels between 1 g/mL and 1000 g/mL. For systemic administration, subjects can be administered a therapeutic amount, such as, e.g., 0.1 mg/kg, 0.5 mg/kg, 1.0 mg/kg, 2.0 mg/kg, 2.5 mg/kg, 5 mg/kg, 7.5 mg/kg, 10 mg/kg, 15 mg/kg, 20 mg/kg, 25 mg/kg, 30 mg/kg, 40 mg/kg, 50 mg/kg, or more. These doses can be administered by one or more separate administrations, or by continuous infusion. For repeated administrations over several days or longer, depending on the condition, the treatment is sustained until, for example, the liver disease is treated, as measured by the methods described above or known in the art. However, other dosage regimens can be useful.

### Combinational Therapy

In one embodiment, the agent or compositions described herein is used as a monotherapy. In one embodiment, the agents described herein can be used in combination with other known agents and therapies for a liver disease. Administered “in combination,” as used herein, means that two (or more) different treatments are delivered to the subject during the course of the subject's affliction with the disorder, e.g., the two or more treatments are delivered after the subject has been diagnosed with the disorder (a liver disease) and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The agents described herein and the at least one additional therapy can be administered simultaneously, in the same or in separate compositions, or sequentially. For sequential administration, the agent described herein can be administered first, and the additional agent can be administered second, or the order of administration can be reversed. The agent and/or other therapeutic agents, procedures or modalities can be administered during periods of active disorder, or during a period of remission or less active disease. The agent can be administered before another treatment, concurrently with the treatment, post-treatment, or during remission of the disorder.

Therapeutics currently used to treat a liver disease include, but are not limited to, ursodeoxycholic acid (UDCA, also known as ursodiol, INN, NAN, AAN, or USAN), cholestyramine, stanozolol, naltrexone, rifampicin, pioglitazone, metformin, rosiglitazone, lobeglitazone, retinol ester, vitamin A, liver dialysis, or liver transplant or any other treatment for liver disease known in the art. In one embodiment, the agent or compositions described herein described herein are not administered with another therapy. Specifically, the agent or compositions described herein described herein are not administered with ursodeoxycholic acid (UDCA, also known as ursodiol, INN, NAN, AAN, or USAN), cholestyramine, stanozolol, naltrexone, rifampicin, pioglitazone, metformin, rosiglitazone, lobeglitazone, retinol ester, vitamin A, liver dialysis, or liver transplant or any other treatment for liver disease known in the art.

When administered in combination, the agent or composition and the additional agent (e.g., second or third agent), or all, can be administered in an amount or dose that is higher, lower or the same as the amount or dosage of each agent used individually, e.g., as a monotherapy. In certain embodiments, the administered amount or dosage of the agent, the additional agent (e.g., second or third agent), or all, is lower (e.g., at least 20%, at least 30%, at least 40%, or at least 50%) than the amount or dosage of each agent used individually. In other embodiments, the amount or dosage of agent, the additional agent (e.g., second or third agent), or all, that results in a desired effect (e.g., treatment of a liver disease) is lower (e.g., at least 20%, at least 30%, at least 40%, or at least 50% lower) than the amount or dosage of each agent individually required to achieve the same therapeutic effect.

### Parenteral Dosage Forms

Parenteral dosage forms of an agents described herein can be administered to a subject by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of a therapeutic agent other than directly into a target site, tissue, or organ, such as a tumor site, such that it enters the subject's circulatory system and, thus, is subject to metabolism and other like processes. In other embodiments, the agent is administered locally, e.g., by direct injections, when the disorder permits, and the injections can be repeated periodically.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

The duration of a therapy using the methods described herein will continue for as long as medically indicated or until a desired therapeutic effect (e.g., those described herein) is achieved. In certain embodiments, the administration of antibody or antigen-binding fragment described herein is continued for 1 month, 2 months, 4 months, 6 months, 8 months, 10 months, 1 year, 2 years, 3 years, 4 years, 5 years, 10 years, 20 years, or for a period of years up to the lifetime of the subject.

### Controlled and Delayed Release Dosage Forms

In some embodiments of the aspects described herein, an agent or composition is administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound of formula (I)'s onset of action, duration of action, plasma levels within the therapeutic window, and peak blood levels. In particular, controlled- or extended-release dosage forms or formulations can be used to ensure that the maximum effectiveness of an agent is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with any agent described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm&Haas, Spring House, Pa. USA).

### Efficacy

The efficacy of an agents described herein, e.g., for the treatment of a liver disease, can be determined by the skilled practitioner. However, a treatment is considered “effective treatment,” as the term is used herein, if one or more of the signs or symptoms of the liver disease are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g., fatigue, pain, weight loss, or dark urine. Efficacy can also be measured by a failure of an individual to worsen as assessed by hospitalization, or need for medical interventions (i.e., progression of the symptoms). Methods of measuring these indicators are known to those of skill in the art and/or are described herein.

Efficacy can be assessed in animal models of a condition described herein, for example, a mouse model or an appropriate animal model of liver disease, as the case may be. When using an experimental animal model, efficacy of treatment is evidenced when a statistically significant change in a marker is observed, e.g., jaundice, fatigue, nausea, vomiting, urine color, abdominal pain.

The term “effective amount” as used herein refers to the amount of an agent or composition described herein can be administered to a subject having or diagnosed as having a liver disease needed to alleviate at least one or more symptom of the disease. The term “therapeutically effective amount” therefore refers to an amount of an agent or composition that is sufficient to provide a particular anti-liver disease effect when administered to a typical subject. An effective amount as used herein, in various contexts, would also include an amount of an agent sufficient to delay the development of a symptom of the disease, alter the course of a symptom of the disease (e.g., slowing the progression of the liver disease), or reverse a symptom of the disease (e.g., correcting or halting symptoms of the liver disease). Thus, it is not generally practicable to specify an exact “effective amount”. However, for any given case, an appropriate “effective amount” can be determined by one of ordinary skill in the art using only routine experimentation.

In one embodiment, the agent or composition is administered continuously (e.g., at constant levels over a period of time). Continuous administration of an agent can be achieved, e.g., by epidermal patches, continuous release formulations, or on-body injectors.

In one embodiment, the agent or composition is administered in intervals (e.g., at various levels over a given period of time).

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the agent, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., measuring liver function, or blood work, among others. The dosage can be determined by a physician and adjusted, as necessary, to suit observed effects of the treatment.

The invention provided herein can further be described in the following numbered paragraphs:
1. A method for treating or preventing a liver disease, the method comprising: administering to a subject in need thereof an antibody or antibody reagent that inhibits WISP1.
2. The method of paragraph 1, wherein the liver disease is selected from the group consisting of: primary biliary cholangitis, autoimmune hepatitis, alpha 1 antitrypsin deficiency, non-alcoholic steatohepatitis, and scleroderma.
3. The method of any preceding paragraph, wherein the WISP1 is a splice variant selected from the group consisting of: WISP1v, WISP1vx, and WISP1delta exon 3-4.
4. The method of any preceding paragraph, wherein the antibody or antibody reagent that inhibits WISP1 is selected from the group consisting of: mab1680, AF1680, SAB2501114, ab60114, and ab65943.
5. The method of any preceding paragraph, wherein the antibody or antibody reagent amino acid sequence comprises at least 70% homology to any one of SEQ ID NOs: 1-4, 6, or 12-120.
6. The method of any preceding paragraph, wherein WISP1 is inhibited in a target cell.
7. The method of any preceding paragraph, wherein the target cell is a mammalian cell.
8. The method of any preceding paragraph, wherein the target cell is a hepatic stellate cell, a fibroblast, or a myofibroblast.
9. The method of any preceding paragraph, wherein the hepatic stellate cell is quiescent.
10. The method of any preceding paragraph, wherein the antibody or antibody reagent is administered by direct injection, subcutaneous injection, muscular injection, or nasal administration.
11. The method of any preceding paragraph, wherein inhibiting WISP1 is inhibiting WISP1 activity or reducing WISP1 protein levels.
12. The method of any preceding paragraph, wherein the activity of WISP1 is inhibited by at least 50%, at least 60%, at least 70%, at least 80%, at least 90%, or more as compared to an appropriate control.
13. The method of any preceding paragraph, wherein the level of WISP1 is reduced by at least 50%, at least 60%, at least 70%, at least 80%, at least 90%, or more as compared to an appropriate control.
14. A composition comprising an antibody or antibody reagent that inhibits WISP1 and a pharmaceutically acceptable carrier.
15. The composition of any preceding paragraph, wherein the antibody or antibody reagent that inhibits WISP1 is selected from the group consisting of: mab1680, AF1680, SAB2501114, ab60114, and ab65943.
16. The composition of any preceding paragraph, wherein the antibody or antibody reagent amino acid sequence comprises at least 70% homology to any one of SEQ ID NOs: 1-4, 6, or 12-120.
17. The composition of any preceding paragraph, wherein the composition is formulated for treating or preventing a liver disease.
18. A method of treating a liver disease in a subject, the method comprising:
a. detecting the level of WISP1 and/or Yap, Colla1, Acta2 in a biological sample of a subject;
b. comparing the measurement of (a) to a reference level;
c. identifying a subject with increased WISP1 and/or Yap, Colla1, Acta2 in (a) as compared to a reference level as having a liver disease; and
d. administering to the subject having liver disease an antibody or antibody reagent that inhibits WISP1.
19. The method of any preceding paragraph, further comprising, prior to (a), obtaining a biological sample from the subject.
20. The method of any preceding paragraph, wherein the liver disease is primary biliary cholangitis, autoimmune hepatitis, alpha 1 antitrypsin deficiency, non-alcoholic steatohepatitis, or scleroderma.
21. The method of any preceding paragraph, wherein the biological sample is a blood sample, tissue, buffy coat, serum, or tissue.
22. The method of any preceding paragraph, wherein the antibody or antibody reagent that inhibits WISP1 is selected from the group consisting of: mab1680, AF1680, SAB2501114, ab60114, and ab65943.
23. The method of any preceding paragraph, wherein the antibody or antibody reagent amino acid sequence comprises at least 70% homology to any one of SEQ ID NOs: 1-4, 6, or 12-120.
24. A method for treating or preventing a liver disease, the method comprising: administering to a subject in need thereof an agent that inhibits WISP1.
25. The method of any preceding paragraph, wherein the liver disease is selected from the group consisting of: primary biliary cholangitis, autoimmune hepatitis, alpha 1 antitrypsin deficiency, non-alcoholic steatohepatitis, and scleroderma.
26. The method of any preceding paragraph, wherein the WISP1 is a splice variant selected from the group consisting of: WISP1v, WISP1vx, and WISP1delta exon 3-4.
27. The method of any preceding paragraph, wherein WISP1 is inhibited in a target cell.
28. The method of any preceding paragraph, wherein the agent that inhibits WISP1 is selected from the group consisting of: a small molecule, an antibody or antibody reagent, a peptide, a genome editing system, a viral vector, a miRNA, and a siRNA.
29. The method of any preceding paragraph, wherein the microRNA is microRNA15a or miRNA412.
30. The method of any preceding paragraph, wherein the antibody or antibody reagent that inhibits WISP1 is selected from the group consisting of: mab1680, AF1680, SAB2501114, ab60114, and ab65943.
31. The method of any preceding paragraph, wherein the antibody or antibody reagent amino acid sequence comprises at least 70% homology to any one of SEQ ID NOs: 1-4, 6, or 12-120.
32. The method of any preceding paragraph, wherein the agent is administered by direct injection, subcutaneous injection, muscular injection, or nasal administration.
33. The method of any preceding paragraph, wherein inhibiting WISP1 is inhibiting WISP1 activity or reducing WISP1 protein levels.
34. The method of any preceding paragraph, wherein the activity of WISP1 is inhibited by at least 50%, at least 60%, at least 70%, at least 80%, at least 90%, or more as compared to an appropriate control.
35. The method of any preceding paragraph, wherein the level of WISP1 is reduced by at least 50%, at least 60%, at least 70%, at least 80%, at least 90%, or more as compared to an appropriate control.
36. A composition comprising an agent that inhibits WISP1 and a pharmaceutically acceptable carrier.
37. The composition of any preceding paragraph, wherein the agent that inhibits WISP1 is selected from the group consisting of: a small molecule, an antibody or antibody reagent, a peptide, a genome editing system, a viral vector, a miRNA, and a siRNA.
38. The composition of any preceding paragraph, wherein the microRNA is microRNA15a or miRNA412.
39. A method of treating a liver disease in a subject, the method comprising:
a. detecting the level of WISP1 and/or Yap, Colla1, Acta2 in a biological sample of a subject;
b. comparing the measurement of (a) to a reference level;
c. identifying a subject with increased WISP1 and/or Yap, Colla1, Acta2 in (a) as compared to a reference level as having a liver disease; and
d. administering to the subject having liver disease an agent that inhibits WISP1.
40. The method of any preceding paragraph, further comprising, prior to (a), obtaining a biological sample from the subject.
41. The method of any preceding paragraph, wherein the liver disease is primary biliary cholangitis, autoimmune hepatitis, alpha 1 antitrypsin deficiency, non-alcoholic steatohepatitis, or scleroderma.
42. The method of any preceding paragraph, wherein the biological sample is a blood sample, tissue, buffy coat, serum, or tissue.
43. The method of any preceding paragraph, wherein the agent that inhibits WISP1 is selected from the group consisting of: a small molecule, an antibody or antibody reagent, a peptide, a genome editing system, a viral vector, a miRNA, and a siRNA.
44. The method of any preceding paragraph, wherein the microRNA is microRNA15a or miRNA412.
45. The method of any preceding paragraph, wherein the antibody or antibody reagent that inhibits WISP1 is selected from the group consisting of: mab1680, AF1680, SAB2501114, ab60114, and ab65943.
46. The method of any preceding paragraph, wherein the antibody or antibody reagent amino acid sequence comprises at least 70% homology to any one of SEQ ID NOs: 1-4, 6, or 12-120.
47. A method of generating an engineered hepatic stellate cell, or population thereof, that expresses an agent that inhibits WISP1, the method comprising; contacting the cell with an agent that inhibits WISP1, and culturing the cell for a sufficient time to allow for expression of the agent.
48. The method of any preceding paragraph, wherein the cell is quiescent.
49. The method of any preceding paragraph, wherein the contacting comprises contacting the cell with an agent or a vector that encodes the agent.
50. The method of any preceding paragraph, wherein the contacting comprises transduction, nucleofection, electroporation, direct injection, and/or transfection.
51. The method of any preceding paragraph, wherein the agent that inhibits WISP1 is selected from the group consisting of: a small molecule, an antibody or antibody reagent, a peptide, a genome editing system, a viral vector, a miRNA, and a siRNA.
52. The method of any preceding paragraph, wherein the microRNA is microRNA15a or miRNA412.
53. The method of any preceding paragraph, wherein the antibody or antibody reagent that inhibits WISP1 is selected from the group consisting of: mab1680, AF1680, SAB2501114, ab60114, and ab65943.
54. The method of any preceding paragraph, wherein the antibody or antibody reagent amino acid sequence comprises at least 70% homology to any one of SEQ ID NOs: 1-4, 6, or 12-120.
55. A cell line comprising hepatic stellate cells generated by the method of any of paragraphs 46-53.
56. A pharmaceutical composition comprising a hepatic stellate cell, or population thereof, generated by the method of any preceding paragraph, and a pharmaceutically acceptable carrier.
57. A method of treating or preventing a liver disease, the method comprising: administering to a subject in need thereof the cells generated by the method of any preceding paragraph, a cell of any preceding paragraph, or the pharmaceutical composition of any preceding paragraph.
58. A method of reducing fibrosis in a subject, the method comprising: administering to a subject in need thereof the cells generated by the method any preceding paragraph, a cell of any preceding paragraph, or the pharmaceutical composition of any preceding paragraph.
59. A method of treating a liver disease in a subject, the method comprising:
a. receiving the results of an assay that identifies a subject as having increased WISP1 and/or Yap, Colla1, Acta2 as compared to a reference level as having a liver disease; and b. administering to the subject having liver disease an antibody or antibody reagent that inhibits WISP1.
60. A method of treating a liver disease in a subject, the method comprising:
a. receiving the results of an assay that identifies a subject as having increased WISP1 and/or Yap, Colla1, Acta2 as compared to a reference level as having a liver disease; and
b. administering to the subject having liver disease an agent reagent that inhibits WISP1.

### EXAMPLES

### Example 1: Treatments for Progressive Liver Diseases

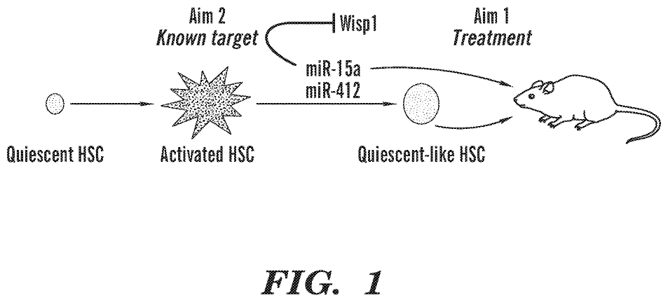

In response to an unmet need for liver disease therapeutics, described herein are microRNAs miR-15a and miR-412 that independently induce quiescence in activated hepatic stellate cells (HSCs), the cell type that plays a central role in fibrotic progression of the liver. Furthermore, miR-15a directly targets WISP1 to repress its pro-fibrotic function in activated HSCs. By promoting HSC quiescence using microRNAs and their target, the microRNAs can be a useful therapy to control progressive hepatic fibrosis in PBC (FIG. 1). Without wishing to be bound by a particular theory, it is contemplated that miR-15a and miR-412 induce quiescence in HSCs which can subsequently elucidate the function of miR-15a's known target WISP1.

Work described herein show quiescent-like HSCs, induced by miR-15a or miR-412, caused improvement of liver damage and fibrosis in CCl4 challenged mice. Given the beneficial effects of miR-15a or miR-412 in CCl4 model, the mouse treatment studies can be expanded using cholestatic fibrosis models, bile duct ligation (BDL) and 3,5-dietoxycarbonyl-1,4-dihydrochollidine (DDC). Three different miRNA delivery systems can be tested: 1) cell therapy using quiescent-like HSCs constitutively expressing miR-15a or miR-412, 2) injecting a lentivirus expressing miR-15a or miR-412 into tail vein, and 3) injecting chemically modified mimics of miR-15a or miR-412 packaged in a lipid based carrier into tail vein. Controls and treatment mice can be used to analyze the outcome.

Work described herein show MiR-15a can directly target WISP1 to repress its pro-fibrotic function in HSCs. The liver phenotype of the WISP1-null mouse in the setting of BDL and DDC challenge can be defined. The severity of liver damage and HSC dysfunction after the challenges can be assessed and compared to that of wildtype mice. Mouse models of cholestatic fibrosis can be treated with WISP1 blocking antibodies and then used to analyze the outcome.

Primary biliary cholangitis (PBC), once named primary biliary cirrhosis, is an insidious liver disease that leads to progressive destruction of intrahepatic bile ducts, cholestasis, periportal inflammation, and eventually biliary fibrosis that ends as cirrhotic end-stage liver disease. It is an autoimmune disorder with unclear pathogenesis although both genetic and environmental factors likely interact to manifest the disease. Interestingly, PBC affects women far more than men with 9:1 female to male disease ratio. The age-adjusted incidence of PBC in the United States per 1 million person-years for women is 45, and 7 for men, while the prevalence per 1 million persons is 654 for women and 121 for men [1].

A hydrophilic bile salt ursodeoxycholic acid (UDCA) is still the only drug that has demonstrated survival benefit for PBC. It has been shown to delay disease progression evidenced by improved histology and biochemical parameters. Although the exact mechanism of its action is unclear, it seems to protect cholangiocytes against toxic bile salts. Unfortunately, 30%-40% of UDCA treated individuals still experience disease progression in the setting of suboptimal response [2]. To fill this treatment gap, many different agents are under clinical trials, but some of the prominently tested drugs such as obeticholic acid still has significant side effects and lacks evidence for survival benefit [3].

Like most chronic liver diseases, PBC progresses from inflammation to fibrosis. During this pathologic cascade, hepatic stellate cells (HSCs) are considered to play a central role in hepatic fibrogenesis [4-6]. HSCs exist in two forms. In healthy individuals, they are quiescent, characterized by multiple retinoid-rich lipid droplets. However, once activated, they lose their lipid droplets and become pro-fibrotic myofibroblasts, secreting collagen and mediators that promote scar formation [7, 8]. Although the importance of HSCs in hepatic fibrosis is well established, there is still much that is unknown about this cell type.

Among multiple gaps in knowledge regarding HSCs, is the current understanding of the role of microRNAs (miRNAs) in determining HSC activation status. MiRNAs are short, non-coding genes that are usually 22 nucleotides in length and are involved in all biologic and pathologic processes. They downregulate specific coding genes by imperfectly base-pairing with complementary sequences within their mRNA targets to induce degradation or translational inhibition. Each miRNA can regulate many different coding genes while each target gene can be regulated by many different miRNAs, constituting a complex layer of gene regulatory network [9]. The experiments described herein have shown that miRNAs are essential for cellular proliferation and reprogramming [10-12]. Nonetheless, miRNAs' role in HSC activation or reversion to quiescence has not been explored thoroughly.

Although groups have profiled global miRNA expression pattern in HSCs the functional significance of their expression status is largely unclear [13-15]. In response, instead of relying on large scale expression profiling, a functional screen has been developed to systematically identify miRNAs that force activated HSCs back toward quiescence, and through this effort, identified miR-15a and miR-412. miR-15a and miR-412 have not been studied in the context of hepatic stellate cell previously. However, miR-15 family was found to inhibit Tgf-beta pathway in the heart to potentially attenuate fibrosis [16]. Promoting HSC quiescence through these miRNAs or their downstream targets can prevent fibrotic progression in PBC. The objective of this study is to assess the therapeutic effect of miR-15a, miR-412, and their direct targets in treating PBC.

### Results

The unbiased functional screen described herein capitalized on the natural tendency of HSCs to become activated when they are grown on plastic surface. As HSCs become activated, they lose lipid droplets present abundantly in quiescent HSCs. Utilizing this useful phenotypic dichotomy, miRNAs that force activated HSCs to become more quiescent-like were searched by tracking the level of lipid droplets that re-form in the cytoplasm. Since miRNAs are known to simultaneously target multiple coding genes to influence an entire cellular program such as organ development, carcinogenesis, or cellular reprogramming [17], it was contemplated that even a single miRNA can induce transdifferentiation.

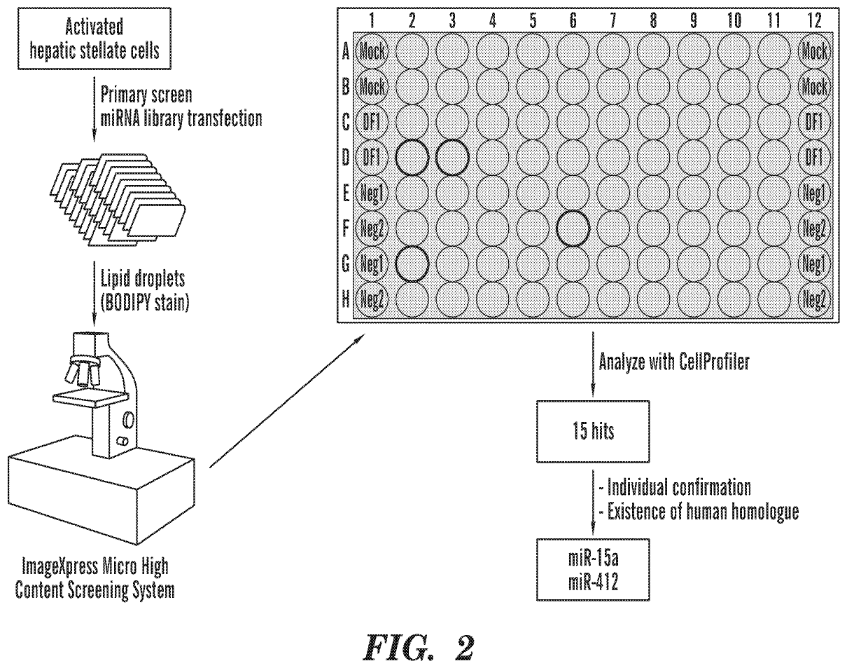

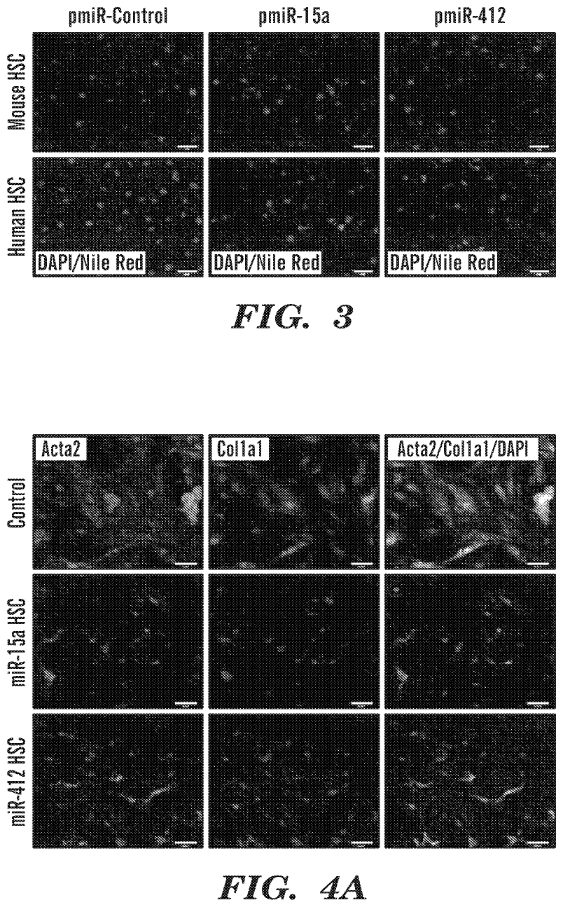

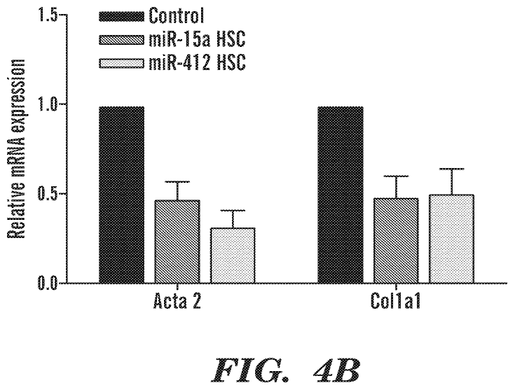

To identify those miRNAs that promote reversion of activated HSCs toward quiescence, reappearance of intracellular lipid droplets with retinoids were scanned for as a marker of quiescence, after activated HSCs received individual miRNA mimics from a full genome miRNA library (FIG. 2). Indeed, HSCs in some of the wells showed Bodipy stain positive lipid droplets re-formed within three days. This initial screen produced 15 primary hits. MiRNAs, miR-15a and miR-412, were chosen for further investigation based on the existence of human orthologues and the ability to re-form lipid droplets in both mouse and human HSCs (FIG. 3). The newly formed lipid droplets were retinoid positive evidenced by fluorescence under ultraviolet light, consistent with those in quiescent HSCs (data not shown). The overall size of the transfected HSCs decreased by 10-100 fold, becoming more quiescent-like (FIG. 4). Furthermore, forced expression of miR-15a or miR-412 downregulated two of the most important gene markers of activation, alpha smooth muscle actin (Acta2) and alpha-1 type I collagen (Col1 a1) (FIG. 4).

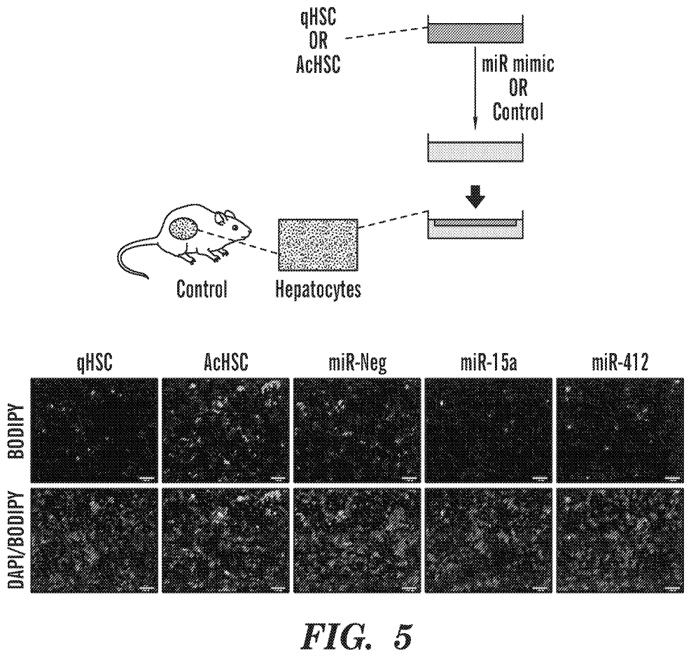

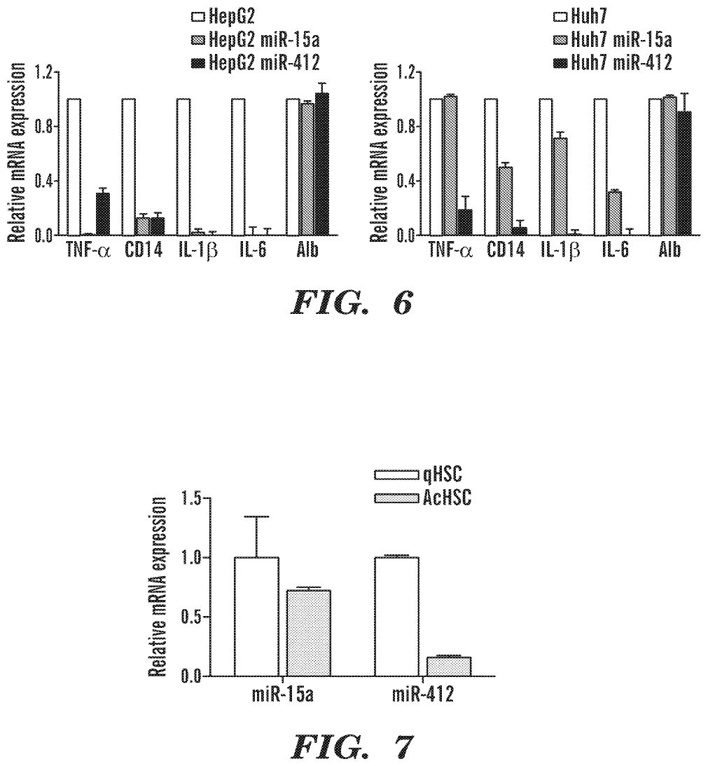

For more comprehensive expression analysis, deep-RNA sequencing demonstrated that the quiescent-like HSCs that received miR-15a or miR-412 had a global transcriptional profile 40-50% closer to quiescent HSCs than activated cells (data not shown). Most importantly, HSCs that became quiescent-like by miR-15a or miR-412 had a functional phenotype similar to truly quiescent HSCs. Ex vivo experiments demonstrated that the quiescent-like HSCs did not induce steatosis in healthy hepatocytes when the two cell types were co-cultured. In contrast, activated HSCs untreated with candidate miRNAs induced hepatocyte steatosis when they were cocultured (FIG. 5). Furthermore, these reverted HSCs were able to decrease pro-inflammatory cytokine expression from cocultured human hepatocellular carcinoma cell lines HepG2 and Huh7 (FIG. 6). Finally, as expected, endogenous miR-15a and miR-412 had decreased expression levels in activated HSCs compared to quiescent HSCs, although not significantly for miR-15a (FIG. 7) and miR-41 seems to force reversion activated HSCs toward quienscence in culture conditions that promote activation. This and other key ex vivo observations allowed for testing whether these miRNAs can be delivered in vivo to attenuate the level of hepatic pathology in the traditional CCl₄ mouse model (oral gavage 100 μl of 40% CCl₄ twice a week), especially since HSCs expressing either miR-15a or miR-412 can maintain a quiescent-like state even in the diseased liver with activation promoting signals. Through cell contact and soluble mediators, quiescent-like HSCs can induce other cell types within the liver to dampen their signals that promote inflammation or fibrosis.

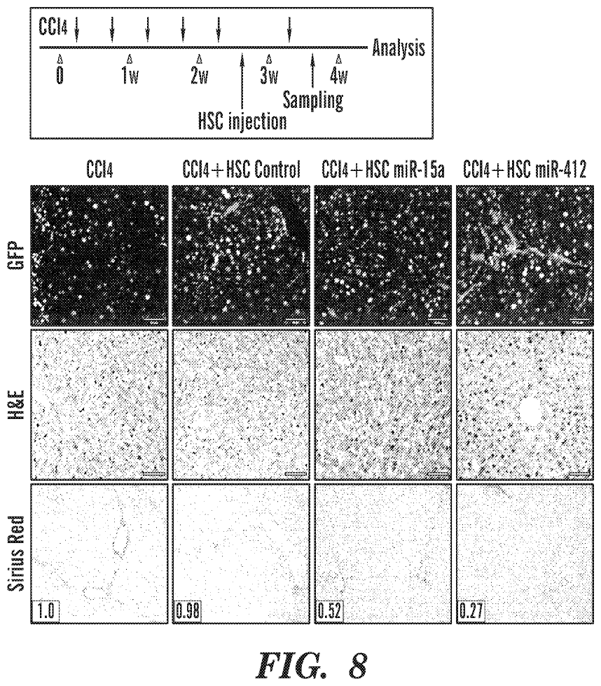

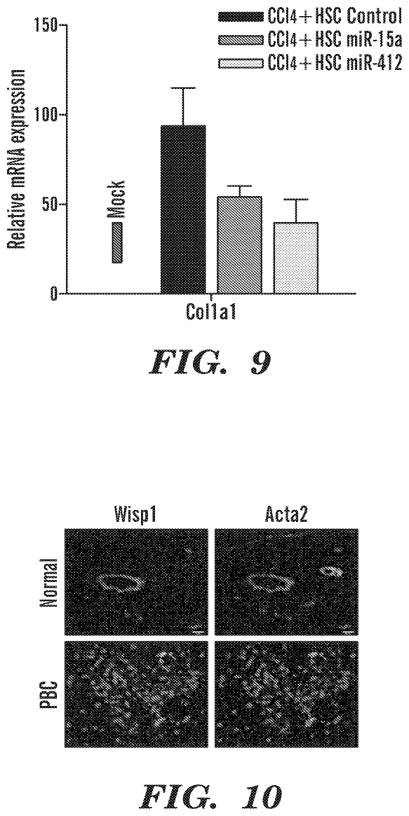

Although there are different methods to deliver miRNAs to live mice, HSCs that were chosen to be injected have become quiescent-like ex vivo by constitutively expressing miR-15a or miR-412. Reprogrammed HSCs were injected once during the third week of four-week CCl₄challenge. Injected HSCs grafted on to the liver, confirmed by visualizing within the liver cells emitting the GFP signal, a piggyBac vector expressing miRNA was inserted [18] (FIG. 8). More importantly, mice treated with quiescent-like HSCs had histology showing decreased ballooning, apoptosis, inflammation, and fibrosis in the liver (FIG. 8) and lower hepatic collagen expression (FIG. 9).

These two miRNA candidates likely downregulate HSC genes that promote hepatic inflammation and fibrosis. Although there are probably many genes repressed by miR-15a and miR-412, WISP1 has experimentally validated direct target of miR-15a. Activated HSCs expressed WISP1 almost 30 times the level of quiescent HSCs (data not shown), and HSCs in human PBC expressed WISP1 much more highly than in normal liver (FIG. 10). Finally, miR-15a is predicted to target WISP1 which contains two potential miR-15a binding sequences within its 3′-UTR. Thus, the putative target sequences or their mutated variants was cloned behind a constitutively active luciferase reporter gene. When one of these reporters were co-transfected with miR-15a mimic, the reporters with wildtype sequences had decreased luciferase expression while the reporters with mutated sequences did not, indicating that miR-15a binds to both WISP1 target sequences (FIG. 11). WISP1 is a key target gene for accomplishing some of the miR-15a actions in HSCs. Testing miR-15a and miR-412 in mouse models of cholangitis.

Delivering miR-15a or miR-412 is a therapy for PBC: Given the surprising results described herein, in vivo experiments were expanded to further assess the potential of miR-15a and miR-412 in treating PBC. To assess whether these miRNAs exert their function solely through HSCs or more broadly through other cell types, miRNAs can be delivered systemically using a viral vector or mimics instead of cell therapy. For all in vivo treatment experiments, the efficacy can be assessed by performing PCR of profibrotic genes in the liver, histology including H&E and Sirius Red stain, hepatic hydroxyproline assay, and measuring plasma ALT and alkaline phosphatase.

Each model of PBC has strengths and weaknesses in representing human PBC. For the purpose of this study, models are needed that consistently progress to biliary fibrosis relatively quickly in order to test the miRNA candidates and inhibition of WISP1 that can lead to decreases in fibrosis and inflammation. Most of the genetic models of PBC do not lead to hepatic fibrosis or do so very slowly [19]. Therefore, two cholangitis models can be utilized that reliably reach fibrosis, surgically induced by bile duct ligation (BDL) and chemically induced by 3,5-dietoxycarbonyl-1,4-dihydrochollidine (DDC). All experiments can be terminated after 3 weeks for BDL model and after 4 weeks for DDC model since these are required times for developing a significant level of cholestatic fibrosis [20-22].

Using mesenchymal stem cells and macrophages, multiple groups have already attempted cell therapy for liver fibrosis with some success [23]. However, the experiments described herein utilize for the first time purposefully engineered HSCs to prevent hepatic fibrosis. Although initial in vivo treatment results are compelling, testing the effect of this cell therapy in mouse models of cholangitis are useful considering only the CCl₄ model has been tested so far. For all cell therapy experiments, injections can be made into the spleen and 500,000 quiescent-like HSCs reprogrammed with miR-15a or miR-412. All appropriate controls, for example, control groups with no treatment and treatment with HSCs that have not been reprogrammed can also be included.

Mice challenged with BDL for 21 days normally develop significant fibrosis. Reprogrammed HSCs can be injected on days 7 and 14 during the 21-day course of BDL challenge. All mice are sacrificed on day 21. Mice challenged with DDC for 28 days normally develop significant fibrosis. Reprogrammed HSCs can be injected on days 14 and 21 during the 28 day course of DDC challenge. All mice are sacrificed on day 28.

Initial experiments demonstrated that injecting HSCs reprogrammed with miR-15a or miR-412 can decrease collagen expression and the overall level of liver damage in CC14 induced hepatic fibrosis. Although delivering miRNAs through cell therapy has shown promise, other delivery methods should be explored. One way is to inject a lentivirus expressing miRNA. The lentivirus can express either miR-15a or miR-412 into the tail vein on days 7 and 14 during the 21-day course of BDL challenge. All mice are sacrificed on day 21. The lentivirus can express either miR-15a or miR-412 on days 14 and 21 during the 28 day course of DDC challenge. All mice are sacrificed on day 28.

As another delivery method, the viability of injecting more stable, chemically modified miRNA mimics (Exiqon and Invitrogen) packaged in a lipid based carrier (MaxSuppressor™ by B100 Scientific) can be tested [24, 25]. By avoiding lentivirus, this method or its variation has the potential of being used in humans. Carriers can be injected containing either miR-15a or miR-412 into the tail vein on days 7 and 14 during the 21 day course of BDL challenge. All mice are sacrificed on day 21. Carriers can be injected containing either miR-15a or miR-412 on days 14 and 21 during the 28 day course of DDC challenge. All mice are sacrificed on day 28.

Since it is difficult to induce hepatic fibrosis in female mice, male mice are more useful for the in vivo treatment studies described herein. A power calculation shows that 8 mice can be used for each group to detect 50% decrease in alpha-1 type I collagen level with 20% standard deviation in the measurement at a p value of 0.01 and 90% power. One of the biggest challenges of treating HSCs with miRNAs is developing a delivery system that specifically targets this cell type to minimize off-target effects. The delivery system can be optimized for improved specificity. For example, two promising HSC delivery systems that were recently developed are a p75 neurotrophin receptor peptide (p75NTRp)-tagged adenovirus and AAV6 vectors [26, 27].

### Assessing the Function of WISP1 in Mouse Models of Cholangitis.

MiR-15a target Wisp1 promotes inflammation and fibrosis in PBC: WISP1 is a member of the Ccn family of matricellular proteins that include Cyr61 (Ccn1), Ctgf (Ccn2), Nov (Ccn3), WISP1 (Ccn4), Wisp2 (Ccn5), and Wisp3 (Ccn6). Ctgf is already established as an important pro-fibrotic factor in the liver [28].

Interestingly, WISP1 was found to be upregulated in human idiopathic pulmonary fibrosis, and treating bleomycin challenged mice with neutralizing WISP1 antibody caused attenuation of lung fibrosis [29]. Moreover, a small animal study showed that blocking WISP1 ameliorates CCl₄ induced liver fibrosis [30]. However, the role of WISP1 in the liver has not been researched in detail, especially in the context of PBC.

Although WISP1 is a direct target of miR-15a, its influence on HSC activation status and its role in PBC pathogenesis is unclear. A WISP1-null mouse line has been generated to demonstrate WISP1's role in bone formation, but its function in the liver was not studied [31]. These mice are fertile and do not have an overt hepatic phenotype. Their mutant allele is preserved in frozen sperm and embryos at an MMRRC facility in University of California at Davis who guarantees successful mutant mouse derivation.

The phenotype can be assessed by the overall severity of hepatic pathology. WISP1-null mice can be challenged with BDL and DDC. The resulting liver damage sustained by WISP1-null mice can be compared with that of wildtype mice. Given the known profibrotic role of Wisp1 in other organs including the lung, WISP1-null mice are expected to have a decreased hepatic fibrosis and perhaps even decreased inflammation. For all in vivo experiments, the liver phenotype can be assessed by performing PCR of profibrotic genes in the liver, histology including H&E and Sirius Red stain, hepatic hydroxyproline assay, and measuring plasma ALT and alkaline phosphatase. Lastly, the functional phenotype of HSCs harvested from these mouse models of biliary fibrosis can be assessed. Male wildtype and WISP1-null mice can be challenged with BDL. They can be sacrificed on day 21. Male wildtype and WISP1-null mice can be challenged with DDC. They can be sacrificed on day 28.

HSCs can then be isolated from mice challenged with 21 days of BDL and 28 days of DDC. These HSCs can be co-cultured with healthy hepatocytes to determine whether pro-inflammatory mediators are induced. The results can be compared to those of wildtype HSCs that underwent the same challenge.

Blocking WISP1 with a neutralizing antibody attenuated bleomycin-induced fibrosis in the mouse lung [29]. Similarly, blocking Wisp1 can decrease the level of inflammation and fibrosis that occur in PBC. Mouse models of cholangitis can be treated with WISP1 antibody and assess the liver phenotype.

Mice challenged with BDL for 21 days normally develop significant fibrosis. The treatment group can receive on days 7 and 14 the commercially available WISP1 antibody previously used for treating bleomycin-induced lung fibrosis (R&D Systems) [29]. The control group can receive IgG. All mice can be sacrificed on day 21. Mice challenged with DDC for 28 days normally develop significant fibrosis. WISP1 blocking antibody can be injected on days 14 and 21 during the 28 day course of DDC challenge. All mice can be sacrificed on day 28.

As described herein, WISP1 is a pro-inflammatory and pro-fibrotic mediator that can be neutralized with an antibody to decrease the level of inflammation and fibrosis in vivo. Therefore, it is contemplated that blocking WISP1 can also ameliorate biliary fibrosis. Therefore, by analyzing the conditioned media of activated HSCs, secreted paracrine or autocrine mediators can be identified. More detailed analysis of the activated HSC conditioned media can be analyzed through mass spectrometry to identify other mediators that are drug targets.

### REFERENCES—EXAMPLE 1

1. Pandit S and Samant H (2017) Primary Biliary Cholangitis (Primary Biliary Cirrhosis). StatPearls, Treasure Island (FL)
2. Pares A, Caballeria L and Rodes J (2006) Excellent long-term survival in patients with primary biliary cirrhosis and biochemical response to ursodeoxycholic Acid. Gastroenterology 130:715-20. doi: 10.1053/j.gastro.2005.12.029
3. Khanna A and Jones DE (2017) Novel strategies and therapeutic options for the management of primary biliary cholangitis. Therap Adv Gastroenterol 10:791-803. doi: 10.1177/1756283X17728669
4. Friedman S L (2008) Mechanisms of hepatic fibrogenesis. Gastroenterology 134:1655-69. doi: 10.1053/j.gastro.2008.03.003
5. Friedman S L (2008) Hepatic stellate cells: protean, multifunctional, and enigmatic cells of the liver. Physiol Rev 88:125-72. doi: 10.1152/physrev.00013.2007
6. Puche J E, Lee Y A, Jiao J, Aloman C, Fiel M I, Munoz U, Kraus T, Lee T, Yee H F, Jr. and Friedman SL (2013) A novel murine model to deplete hepatic stellate cells uncovers their role in amplifying liver damage in mice. Hepatology 57:339-50. doi: 10.1002/hep.26053
7. Kisseleva T, Cong M, Paik Y, Scholten D, Jiang C, Benner C, Iwaisako K, Moore-Morris T, Scott B, Tsukamoto H, Evans S M, Dillmann W, Glass C K and Brenner D A (2012) Myofibroblasts revert to an inactive phenotype during regression of liver fibrosis. Proc Natl Acad Sci USA 109:9448-53. doi: 10.1073/pnas.1201840109
8. Mederacke I, Hsu C C, Troeger J S, Huebener P, Mu X, Dapito D H, Pradere J P and Schwabe R F (2013) Fate tracing reveals hepatic stellate cells as dominant contributors to liver fibrosis independent of its aetiology. Nat Commun 4:2823. doi: 10.1038/ncomms3823
9. Szabo G and Csak T (2016) Role of MicroRNAs in NAFLD/NASH. Dig Dis Sci. doi: 10.1007/s10620-015-4002-4
10. Kim B M, Thier M C, Oh S, Sherwood R, Kanellopoulou C, Edenhofer F and Choi M Y (2012) MicroRNAs are indispensable for reprogramming mouse embryonic fibroblasts into induced stem cell-like cells. PLoS One 7:e39239.
11. Kanellopoulou C, Muljo S A, Kung A L, Ganesan S, Drapkin R, Jenuwein T, Livingston D M and Rajewsky K (2005) Dicer-deficient mouse embryonic stem cells are defective in differentiation and centromeric silencing. Genes Dev 19:489-501.
12. Kim B M and Choi M Y (2012) Non-canonical microRNAs miR-320 and miR-702 promote proliferation in Dgcr8-deficient embryonic stem cells. Biochem Biophys Res Commun 426:183-9.
13. Guo C J, Pan Q, Cheng T, Jiang B, Chen G Y and Li D G (2009) Changes in microRNAs associated with hepatic stellate cell activation status identify signaling pathways. FEBS J 276:5163-76. doi: 10.1111/j.1742-4658.2009.07213.x
14. Maubach G, Lim M C, Chen J, Yang H and Zhuo L (2011) miRNA studies in in vitro and in vivo activated hepatic stellate cells. World J Gastroenterol 17:2748-73. doi: 10.3748/wjg.v17.i22.
15. Lakner A M, Steuerwald N M, Walling T L, Ghosh S, Li T, McKillop I H, Russo M W, Bonkovsky H L and Schrum L W (2012) Inhibitory effects of microRNA 19b in hepatic stellate cell-mediated fibrogenesis. Hepatology 56:300-10. doi: 10.1002/hep.25613
16. Tijsen A J, van der Made I, van den Hoogenhof M M, Wijnen W J, van Deel E D, de Groot N E, Alekseev S, Fluiter K, Schroen B, Goumans M J, van der Velden J, Duncker D J, Pinto Y M and Creemers E E (2014) The microRNA-15 family inhibits the TGFbeta-pathway in the heart. Cardiovasc Res 104:61-71. doi: 10.1093/cvr/cvu184
17. Ivey K N and Srivastava D (2010) MicroRNAs as regulators of differentiation and cell fate decisions. Cell Stem Cell 7:36-41. 18. Griffin T A, Anderson H C and Wolfe J H (2015) Ex vivo gene therapy using patient iPSC-derived NSCs reverses pathology in the brain of a homologous mouse model. Stem Cell Reports 4:835-46. doi: 10.1016/j.stemcr.2015.02.022
19. Katsumi T, Tomita K, Leung P S, Yang G X, Gershwin M E and Ueno Y (2015) Animal models of primary biliary cirrhosis. Clin Rev Allergy Immunol 48:142-53. doi: 10.1007/s12016-015-8482-y
20. Fickert P, Stoger U, Fuchsbichler A, Moustafa T, Marschall H U, Weiglein A H, Tsybrovskyy O, Jaeschke H, Zatloukal K, Denk H and Trauner M (2007) A new xenobiotic-induced mouse model of sclerosing cholangitis and biliary fibrosis. Am J Pathol 171:525-36. doi: 10.2353/ajpath.2007.061133
21. Georgiev P, Jochum W, Heinrich S, Jang J H, Nocito A, Dahm F and Clavien P A (2008) Characterization of time-related changes after experimental bile duct ligation. Br J Surg 95:646-56. doi: 10.1002/bjs.6050
22. Pollheimer M J and Fickert P (2015) Animal models in primary biliary cirrhosis and primary sclerosing cholangitis. Clin Rev Allergy Immunol 48:207-17. doi: 10.1007/s12016-014-8442-y
23. Terai S and Tsuchiya A (2017) Status of and candidates for cell therapy in liver cirrhosis: overcoming the “point of no return” in advanced liver cirrhosis. J Gastroenterol 52:129-140. doi: 10.1007/s00535-016-1258-1
24. Trang P, Wiggins J F, Daige C L, Cho C, Omotola M, Brown D, Weidhaas J B, Bader A G and Slack F J (2011) Systemic delivery of tumor suppressor microRNA mimics using a neutral lipid emulsion inhibits lung tumors in mice. Mol Ther 19:1116-22. doi: 10.1038/mt.2011.48
25. Wiggins J F, Ruffino L, Kelnar K, Omotola M, Patrawala L, Brown D and Bader A G (2010) Development of a lung cancer therapeutic based on the tumor suppressor microRNA-34. Cancer Res 70:5923-30. doi: 10.1158/0008-5472.CAN-10-0655
26. Song G, Pacher M, Balakrishnan A, Yuan Q, Tsay H C, Yang D, Reetz J, Brandes S, Dai Z, Putzer B M, Arauzo-Bravo M J, Steinemann D, Luedde T, Schwabe R F, Manns M P, Scholer H R, Schambach A, Cantz T, Ott M and Sharma A D (2016) Direct Reprogramming of Hepatic Myofibroblasts into Hepatocytes In Vivo Attenuates Liver Fibrosis. Cell Stem Cell 18:797-808. doi: 10.1016/j.stem.2016.01.010
27. Rezvani M, Espanol-Suner R, Malato Y, Dumont L, Grimm A A, Kienle E, Bindman J G, Wiedtke E, Hsu B Y, Naqvi S J, Schwabe R F, Corvera C U, Grimm D and Willenbring H (2016) In Vivo Hepatic Reprogramming of Myofibroblasts with AAV Vectors as a Therapeutic Strategy for Liver Fibrosis. Cell Stem Cell 18:809-16. doi: 10.1016/j.stem.2016.05.005
28. Chen L, Charrier A, Zhou Y, Chen R, Yu B, Agarwal K, Tsukamoto H, Lee L J, Paulaitis M E and Brigstock D R (2014) Epigenetic regulation of connective tissue growth factor by MicroRNA-214 delivery in exosomes from mouse or human hepatic stellate cells. Hepatology 59:1118-29. doi: 10.1002/hep.26768
29. Konigshoff M, Kramer M, Balsara N, Wilhelm J, Amarie O V, Jahn A, Rose F, Fink L, Seeger W, Schaefer L, Gunther A and Eickelberg 0 (2009) WNT1-inducible signaling protein-1 mediates pulmonary fibrosis in mice and is upregulated in humans with idiopathic pulmonary fibrosis. J Clin Invest 119:772-87. doi: 10.1172/JC133950
30. Li X, Chen Y, Ye W, Tao X, Zhu J, Wu S and Lou L (2015) Blockade of CCN4 attenuates CCl₄-induced liver fibrosis. Arch Med Sci 11:647-53. doi: 10.5114/aoms.2015.52371
31. Maeda A, Ono M, Holmbeck K, Li L, Kilts™, Kram V, Noonan M L, Yoshioka Y, McNerny E M, Tantillo M A, Kohn D H, Lyons K M, Robey P G and Young M F (2015) WNT1-induced Secreted Protein-1 (WISP1), a Novel Regulator of Bone Turnover and Wnt Signaling. J Biol Chem 290:14004-18. doi: 10.1074/jbc.M114.628818.

### Example 2: Treatments for Non-Alcoholic Fatty Liver Disease (NAFLD)

Non-alcoholic fatty liver disease (NAFLD) can become the most common cause of end-stage liver disease, hepatic transplant, and hepatocellular carcinoma in the developed world in the next 5-10 years. Currently, reducing risk factors that can lead to NAFLD is the main mode of management as no FDA approved drug exists. Thus, developing effective therapies for NAFLD is of utmost importance.

Activated hepatic stellate cells (HSCs) are known to play a pivotal role in fibrotic progression of the liver, and the data presented herein demonstrate that microRNAs miR-15a and miR-412 independently revert activated HSCs back toward quiescence. Furthermore, miR-15a directly targets WISP1 to repress its pro-steatotic and pro-inflammatory function in activated HSCs. Wishing to not be bound by a particular theory, it was hypothesized that miR-15a and miR-412 promote quiescence in HSCs which can subsequently suppress fibrotic progression in NAFLD.

With the advent of highly effective antiviral therapies against hepatitis B and C, non-alcoholic fatty liver disease (NAFLD) has become the most serious hepatic disorder. NAFLD is now considered the most common liver disease in the developed world [1], and is projected to become the leading cause of end-stage liver disease, hepatic transplant, and hepatocellular carcinoma by 2025 [2, 3]. The disease is particularly common in rich nations such as the United States where 64 million people are estimated to be affected due to high prevalence of risk factors including obesity, diabetes, and hyperlipidemia. Moreover, the economic burden of NAFLD is staggering, with annual direct medical costs projected to be about $103 billion [4]. The disease starts as hepatic steatosis but can progress to non-alcoholic steatohepatitis (NASH), fibrosis, and ultimately cirrhotic liver failure or hepatocellular carcinoma. NAFLD is currently managed mainly by decreasing risk factors. However, these measures are often difficult to achieve, and even eliminating them does not guarantee improvement [5]. Given that NAFLD is becoming the most common cause of liver related morbidity and that there are no drugs to manage it currently, better treatment options are necessary. As described herein, are the first steps in developing new therapies for NAFLD.

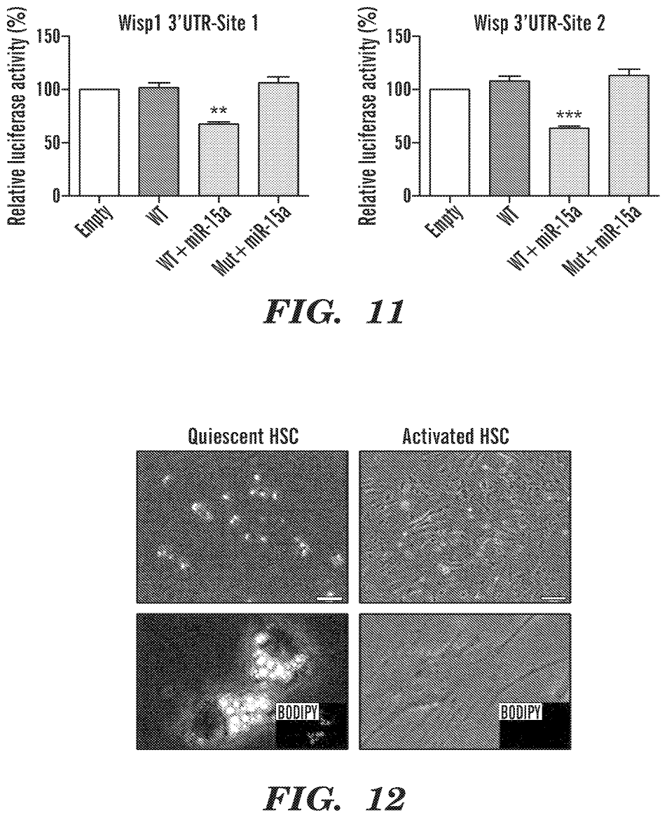

NAFLD progresses in a stepwise fashion through steatosis, inflammation, and fibrosis. During this pathologic cascade, hepatic stellate cells (HSCs) are considered to play a central role in hepatic fibrogenesis [6-8]. HSCs exist in two forms. In healthy individuals, they are quiescent, characterized by multiple retinoid-rich lipid droplets (FIG. 12). However, once activated, they lose their lipid droplets and become pro-fibrotic myofibroblasts, secreting collagen and mediators that promote scar formation (FIG. 12) [9, 10].

Although the importance of HSCs in hepatic fibrosis is well established, there is still much that is unknown about this cell type. For example, are there genes that can reverse activated HSCs back toward quiescence?Are HSCs also involved in early NAFLD, promoting steatosis and inflammation, not just fibrosis?If HSCs contribute to steatohepatitis or fibrosis, what are the important mediators involved in these processes?Answering these questions and gaining better biological insight can lead to novel ideas for managing NAFLD.

Among many gaps in current knowledge, is the understanding of the role of microRNAs (miRNAs) in determining HSC activation status is particularly poor. Although groups have profiled global miRNA expression pattern in HSCs the functional significance of their expression status is largely unclear [11-13]. In response, research has focused on first determining miRNA function, discovering key miRNAs that influence HSC's activation status. Instead of relying on large scale expression profiling, a functional screen and assays have been developed to systematically identify miRNAs that force activated HSCs back toward quiescence, and through this effort, identified miR-15a and miR-412. Promoting HSC quiescence through these miRNAs or their downstream targets can prevent fibrotic progression in NAFLD.

Finally, activated HSCs causing fibrosis is not unique to NAFLD. Most chronic liver diseases involve HSCs promoting fibrosis for years before arriving at cirrhosis. Thus, if a therapeutic agent can decrease the rate of fibrotic progression by targeting HSCs, it is possible to use this approach in other liver diseases besides NAFLD. The potential impact of such a therapy is enormous. Even for diseases such as primary biliary cirrhosis or autoimmune hepatitis that have established drug therapies, an additional agent that inhibits fibrotic progression could significantly improve the ability to manage these chronic illnesses. In conclusion, given both the importance of NAFLD and the central role HSCs play in its fibrotic progression, the significance of the research described herein is wide-ranging.

Although several compounds are in human trials for treating NAFLD, none of them rely on preventing or reversing HSC activation as the primary goal [5, 14, 15]. In response, the first major innovation started with the attempt to control the activation status of HSCs as a way to prevent progressive liver damage and fibrosis. The in vitro screen described herein capitalized on the natural tendency of HSCs to become activated when they are grown on plastic surface. As HSCs become activated, they lose lipid droplets present abundantly in quiescent HSCs (FIG. 12). Utilizing this useful phenotypic dichotomy, miRNAs that force activated HSCs to become more quiescent-like by were searched for tracking the level of lipid droplets that re-form in the cytoplasm. Since miRNAs are known to simultaneously target multiple coding genes to influence an entire cellular program such as organ development, carcinogenesis, or cellular reprogramming [16], it was reasoned that even a single miRNA may be able to induce this transdifferentiation. Indeed, through an unbiased survey, several miRNA candidates were identified that seem to revert HSCs back toward quiescence from the activated state, demonstrated by the reappearance of intracellular lipid droplets (FIG. 3). A subset of these miRNAs also caused other phenotypic changes toward HSC quiescence including gene expression pattern, proliferation rate, and influence on nearby hepatocytes. This innovation in utilizing miRNAs to control HSC activation status serves as a foundation for developing an entirely new category of therapy that prevents hepatic fibrosis.

The second major innovation comes in the form of a novel cell therapy using quiescent-like HSCs. Although the initial screen and follow-up in vitro assays were done using transient transfection with mimics of miR-15a or miR-412, when a piggyBac transposon vector was then used that can integrate into the genome to constitutively express either miRNA in HSCs [17], they seem to transdifferentiate permanently toward a quiescent-like state accompanied by dramatic changes in morphology, expression, and function. Strikingly, when these reprogrammed HSCs were injected into mice challenged with 0014, they engrafted on to liver and improved the level of liver damage and fibrosis. Other groups have attempted cell therapy in liver cirrhosis using mesenchymal stem cells or macrophages [18], but this is the first time engineered HSCs were used as a therapy for hepatic fibrosis or NASH.

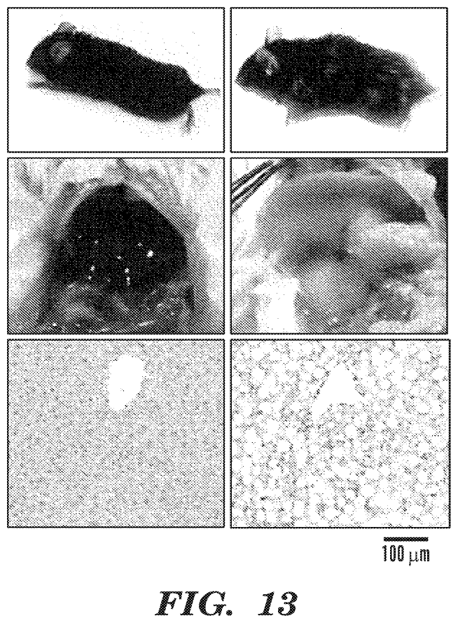

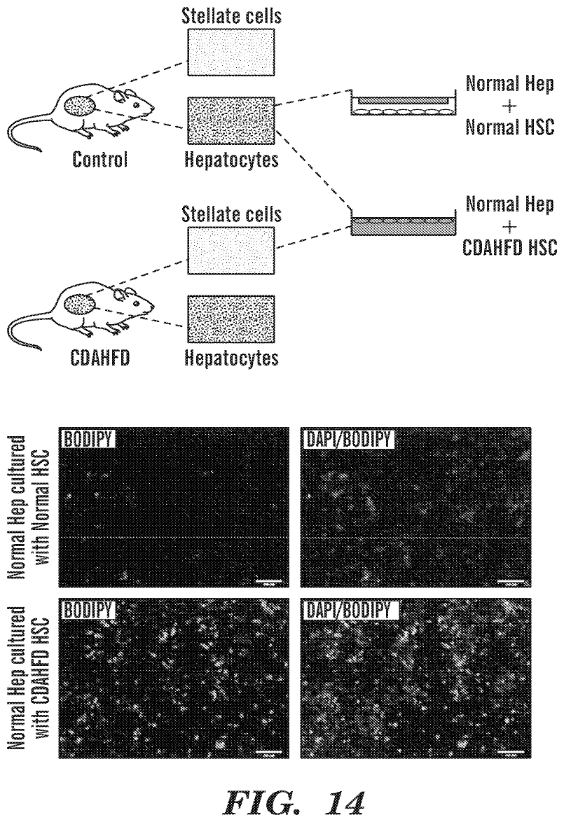

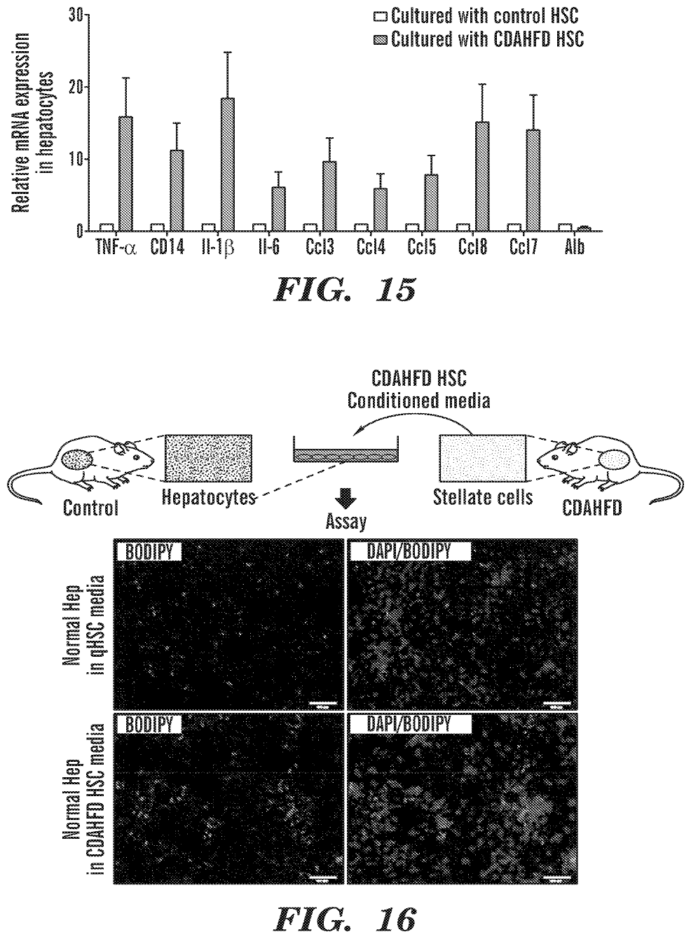

The last major innovation is technical in nature. If HSCs are already known to be the main driver of hepatic fibrosis in NASH, it is possible to disrupt their action. However, there are fundamental challenges to this endeavor. One of the biggest obstacles that hinder the study of HSC function and mechanism of action is the paucity of good in vitro models for HSC's interaction with other cell types, allowing simple assays to assess functional phenotype and mechanism of action. To meet this need, a coculture system using primary mouse HSCs and hepatocytes were developed to recreate the hepatic microenvironment of these two cell types in close proximity. Moreover, by combining this technology with mouse models of NASH, the effect of NASH-HSCs on nearby hepatocytes can be reliably measured. Interestingly, NASH-HSCs from choline deficient, L-amino acid defined, high fat diet (CDAHFD model (FIG. 13) induced steatosis and stimulated expression of pro-inflammatory cytokines in cocultured hepatocytes harvested from healthy mice (FIG. 14, 15) [19]. This signaling does not require cell-cell contact because the two cell types in coculture can be separated by a transwell, implying secretion of soluble factor(s) by HSCs. In fact, induction of fatty accumulation in hepatocytes can be reproduced even when the conditioned media from cultured NASH-HSCs is applied to normal hepatocytes, without coculture (FIG. 16). Thus, this system allows for identification of HSC-secreted mediators that induce steatosis and pro-inflammatory cytokines in hepatocytes. By modeling the interaction between HSCs and hepatocytes in NAFLD using a simple, yet robust, ex vivo system, a technology has been created that is amenable to mechanistic and drug discovery experiments. In particular, by utilizing primary cells instead of cell lines, this coculture system more faithfully simulates the pathophysiologic microenvironment of these two cell types in vivo [20]. The coculture system is feasible by improving existing protocols for harvesting and culturing these cells so that the quality is maximized while the effort is minimized [21]. These technical enhancements are useful for delivering consistent and reproducible results using primary cells. Defining the mechanism and extent to which miR-15a and miR-412 influence HSC activation.

Hepatic stellate cells (HSCs) make up only 5% to 15% of all cells in the liver, but their enormous pro-fibrotic influence after their activation in progressive liver diseases is well recognized [6, 7, 23, 24]. Although several studies demonstrate differential miRNA expression in activated versus quiescent HSCs [11-13], the function of miRNAs during the activation process is still largely unclear. Even less understood is the role of miRNAs in the reversion of activated HSCs back toward quiescence, one possible fate of HSCs during the resolution of hepatic inflammation and fibrosis [9, 25].

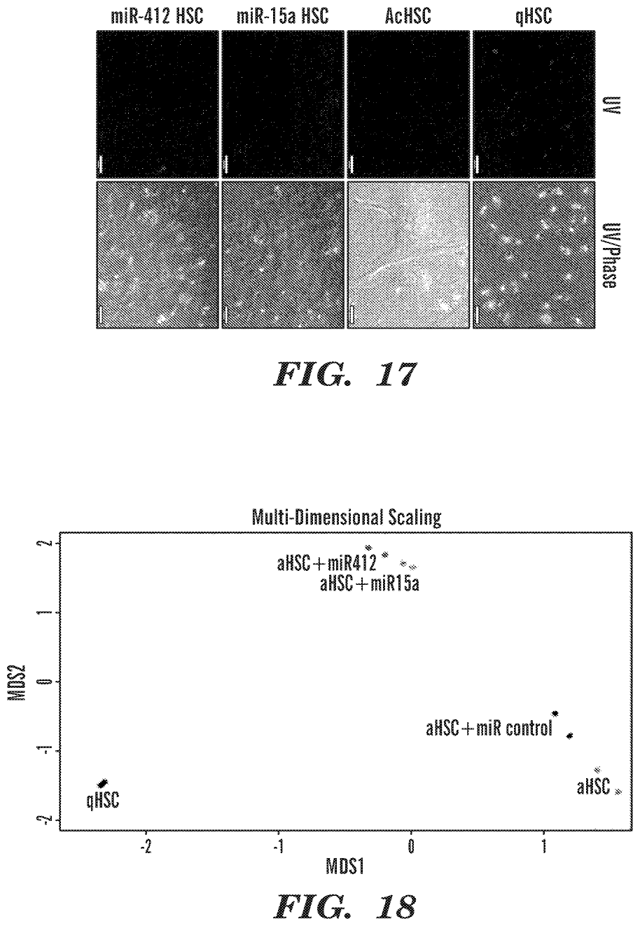

To identify those miRNAs that promote reversion of activated HSCs toward quiescence, an unbiased functional screen was designed to look for reappearance of intracellular lipid droplets with retinoids, a marker of quiescence, after activated HSCs received individual miRNA mimics from a full genome miRNA library (FIG. 2). A survey was performed in 96 well plates, culturing activated HSCs with almost no lipid droplets. Once individual miRNAs were transfected into each well, HSCs in some of the wells showed Bodipy stain positive lipid droplets re-formed within three days. This initial screen produced 15 primary hits. miRNAs miR-15a and miR-412 were chose for further investigation based on the existence of human orthologues and the ability to re-form lipid droplets in both mouse and human HSCs (FIG. 3). The newly formed lipid droplets were retinoid positive evidenced by fluorescence under ultraviolet light, consistent with those in quiescent HSCs (FIG. 17). The overall size of the transfected HSCs decreased by 10-100 fold, becoming more quiescent-like (FIG. 4). Furthermore, forced expression of miR-15a or miR-412 downregulated two of the most important gene markers of activation, alpha smooth muscle actin (Acta2) and alpha-1 type (Colla1) (FIG. 4).

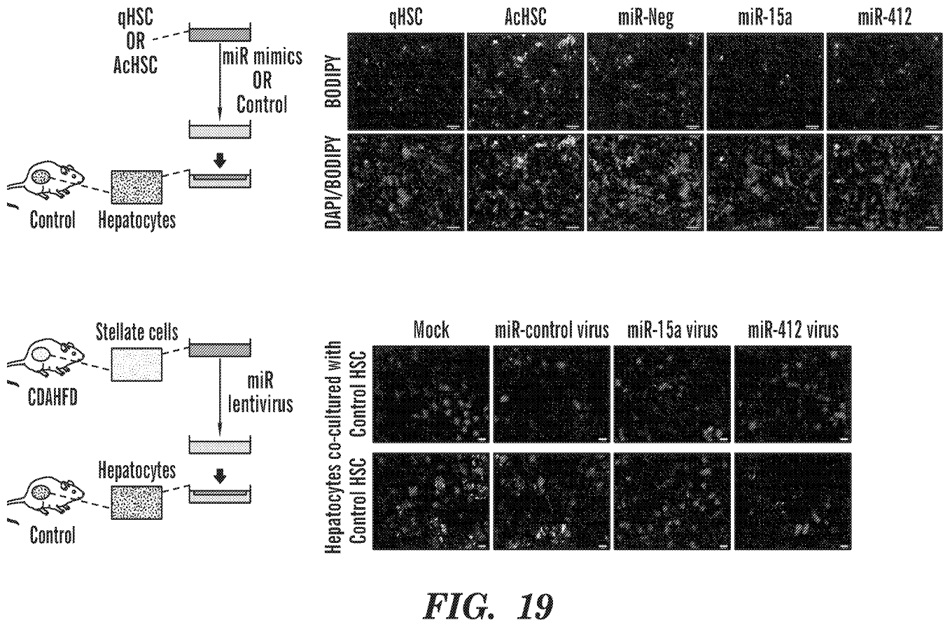

For more comprehensive expression analysis, deep-RNA sequencing demonstrated that the quiescent-like HSCs that received miR-15a or miR-412 had a global transcriptional profile 40-50% closer to quiescent HSCs than activated cells (FIG. 18). Most surprisingly, HSCs that became quiescent-like by miR-15a or miR-412 had a functional phenotype similar to truly quiescent HSCs. Ex vivo experiments demonstrated that the quiescent-like HSCs did not induce steatosis in healthy hepatocytes when the two cell types were cocultured. In contrast, activated HSCs or NASH-HSCs untreated with candidate miRNAs induced hepatocyte steatosis when they were cocultured (FIG. 19). Finally as expected, endogenous miR15a and miR-412 had decreased expression levels in activated HSCs compared to quiescent HSCs, although not significantly for miR-15a (FIG. 7).

MiR-15a and miR-412 have not been studied in the context of hepatic stellate cell. However, miR-15 family was found to inhibit Tgf-beta pathway in the heart to potentially attenuate fibrosis [26], and it is also known as a critical tumor suppressor in chronic lymphocytic leukemia [27, 28]. By contrast, no specific literature exists for miR-412, and thereby its function is completely unknown. MiRNAs, generally, are short, non-coding genes that are usually 22 nucleotides in length and are involved in all biologic and pathologic processes. They downregulate target coding genes by imperfectly base-pairing with complementary sequences within their mRNA targets to induce degradation or translational inhibition. Each miRNA can regulate many different coding genes while each target gene can be regulated by many different miRNAs, constituting a complex layer of gene regulatory network. miRNAs directly target about 50% of all mammalian coding genes, demonstrating their wide reach in gene regulation [29]. It has shown that miRNAs are essential for cellular proliferation and reprogramming [30-32]. Nonetheless, miRNAs' role in HSC activation or reversion to quiescence has not been explored.

As demonstrated herein, is a compelling quiescence-promoting effect of miR-15a and miR-412 in HSCs, and a mechanism of action. WISP1 was identified as a direct target of miR-15a, but there are likely other relevant targets for both miR-15a and miR-412. Numerous prediction algorithms forecast potential direct targets of miRNAs based on sequence complementarities, but most of these predicted targets are not actual targets that can withstand experimental verification. Hence, RNA-Seq of HSCs was performed in various activation states to define the expression profile that can identify true direct targets of miR-15a and miR-412.

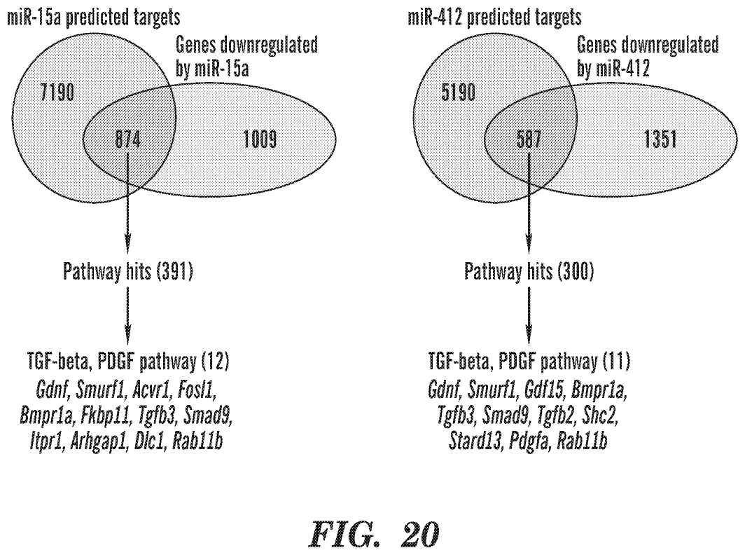

Three types of HSCs were deep-sequenced: 1) quiescent HSCs freshly harvested from healthy mice, 2) activated HSCs that have been passed on cell culture dish multiple times, and 3) quiescent-like HSCs that have reverted toward quiescence from the activated state by receiving either miR-15a or miR-412. Since quiescent-like HSCs were sequenced just three days after receiving either miR-15a or miR-412 mimic, any coding gene's decrease in mRNA level could be the result of direct miRNA targeting. Hence, the set of genes with decreased mRNA level after receiving either miR-15a or miR-412 were overlaid with the set of potential direct targets based on a prediction algorithm [33] (FIG. 20). This new set (from now called the target candidate set) of common genes derived from two parent sets has a higher probability of including true direct targets.

Since the target candidate set still has too many genes to analyze, target candidate genes were chosen that are known to be part of the two best known signaling pathways that promote HSC activation, those of Tgf-beta and Pdgf. When PANTHER analysis was used to filter genes that are part of these two pathways, 12 and 11 target candidates were left for miR-15a and miR-412, respectively (FIG. 20). [34]. These genes can be tested by constructing reporters with or without mutant miRNA binding sequence cloned behind a constitutively active luciferase gene. One of these reporter constructs and a miRNA mimic can be co-transfected into either 293 cells or primary HSCs. If the co-transfected miRNA can anneal to this sequence and repress the translation of luciferase protein, it can indicate that the coding gene is a true direct target of the tested miRNA. The mutant binding sequence should prevent the miRNA from annealing and allow near normal expression of luciferase, further supporting that the tested coding gene sequence is a true miRNA target. It is expected that this experiment is time consuming since 23 high-potential target candidate gene sequences in both actual and mutated forms need to be cloned into the luciferase vector. However, finding direct targets is irreplaceable in elucidating the mechanism of action for miRNAs [32].

So far, the effects of delivering miR-15a or miR-412 to activated HSCs has been described herein. To further understand the function of miR-15a and miR-412, a definitive loss of function experiment can be completed by deleting these genes in primary HSCs using the CRISPR technology as described previously [35].

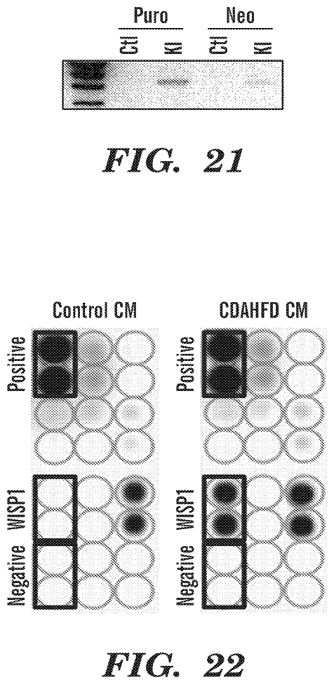

The viability of using this technology on primary HSCs has been verified by delivering digit deletion vectors utilized in the past (FIG. 21) [35]. Three targeted HSC lines can be generated: 1) miR-15a-null, 2) miR-412-null, and 3) miR-15a/miR-412-null. Since both of these miRNAs promoted HSC quiescence with overexpression, it is expected that their deletion can cause an activated phenotype, especially in the miR-15a/miR-412-double null line. A lack of distinct phenotype is also possible since miRNAs are known to have many functional redundancies. The level of activation can be determined by defining their global expression through RNA-Seq and comparing to both activated and quiescent HSCs. The morphology and proliferation can be assessed while cultured on plastic and Matrigel. Activated wildtype HSCs are known to revert back to quiescence on Matrigel [36], and knockout HSCs' phenotype can be characterized in this environment. If miR-15a or miR-412 deletion contributes to HSC activation, this effect can overcome the quiescence-promoting effect of Matrigel. The miRNA-deleted HSCs' interaction with hepatocytes can be evaluated with the coculture system. Activated HSCs cause cocultured hepatocytes to become steatotic and to express pro-inflammatory mediators. The miRNA-deleted HSCs can also be assessed to have a pro-steatotic phenotype even greater than that of wildtype HSCs that are activated. Finally, reconstituting miR-15a or miR-412 can be tested in the respective deletion lines can rescue the wildtype phenotype. The reconstitution of the deleted miRNA can be achieved by transfecting the miRNA-expressing piggyBac vectors have already been cloned and have used for gain of function experiments.

Although miR-15a or miR-412 has been expressed individually, it is also specifically contemplated herein to express them together in the same HSC. Moreover, delivering just one of these miRNAs can shift global gene expression pattern about halfway back toward quiescence from the activated state along the transcriptional axis connecting them (FIG. 11). Overexpressing both miRNAs may revert activated HSCs back toward quiescence even more closely than expressing either miRNA alone. This gain of function experiment can be performed by cloning both miRNAs in a single piggyBac vector and transfecting it into activated HSCs. The piggyBac vector can allow integration of miRNA genes into the genome, constitutively expressing them [17]. HSCs that are simultaneously expressing both miRNAs can be phenotyped with the similar parameters described herein to characterize other HSC lines, including global gene expression using RNA-Seq, morphology, proliferation using an MTT assay, and interaction with hepatocytes using this coculture method.

If the phenotype of primary HSCs expressing both miRNAs even more closely resembles quiescent HSCs, cell therapy can be tested in the setting of CCl₄ induced hepatic fibrosis and diet induced NASH as HSCs overexpressing a single miRNA. This HSC line can prevent or decrease liver damage and fibrosis even more than what was observed with HSC lines expressing either miR-15a or miR-412 alone.

Testing miR-15a or miR-412 Delivery to Mouse Models of Hepatic Fibrosis or NAFLD

Treating HSCs from a diet model of NASH with miR-15a or miR-412 and reverting them back toward quiescence in hepatocytes (FIG. 12). Furthermore, miR-15a and miR-412 seem to force reversion of activated HSCs toward quiescence even in culture conditions that promote activation. These key ex vivo observations allowed testing whether these miRNAs can be delivered in vivo to attenuate the level of hepatic pathology in the traditional CCl₄ mouse model, especially since HSCs expressing either miR-15a or miR-412 can maintain a quiescent-like state even in the diseased liver with activation promoting signals. Through cell contact and soluble mediators, quiescent-like HSCs may induce other cell types within the liver to dampen their signals that promote steatosis, inflammation, or fibrosis.

Although there are different methods to deliver miRNAs to live mice, the HSCs that were injected became quiescent-like by constitutively expressing miR-15a or miR-412. Hence, the viability of a new cell therapy for liver disease was tested further using HSCs that have been engineered to a quiescent-like state. This experiment was performed by injecting reprogrammed HSCs once during the third week of four-week CCl₄ challenge. Injected HSCs grafted on to the liver, confirmed by visualizing within the liver cells emitting the GFP signal that was inserted into the piggyBac vector expressing miRNA [17] (FIG. 8). More importantly, mice treated with quiescent-like HSCs had histology showing decreased ballooning, apoptosis, inflammation, and fibrosis in the liver (FIG. 8) and lower hepatic collagen expression (FIG. 9). Given this observation, in vivo experiments can be expanded to further assess the potential of miR-15a and miR-412 in treating hepatic steatosis, inflammation, and fibrosis using additional mouse models of hepatic fibrosis and NASH. To assess whether these miRNAs exert their function solely through HSCs or more broadly through other cell types, miRNAs can be delivered systemically using a viral vector or mimics instead of cell therapy. For all in vivo treatment experiments, the efficacy can be assessed by performing PCR of profibrotic genes in the liver, histology including H&E and Sirius Red stain, hepatic hydroxyproline assay, and measuring plasma ALT.

Using mesenchymal stem cells and macrophages, multiple groups have already performed cell therapy for liver fibrosis with some success [18]. However, these experiments utilize for the first time purposefully engineered HSCs to prevent or reverse hepatic fibrosis. The HSCs originated from separate, congenic mice, and 500,000 cells were injected once on the third week of a four week course of CCl₄. Although the initial results are compelling, it is unclear whether the improvement in liver histology stems from prevention of liver damage provoked on the fourth week of CCl₄ administration or from reversal of the damage incurred during the first three weeks of CCl₄ challenge. The effect of this cell therapy in NASH is not well understood, considering that only the CCl₄ model has been used thus far. Finally, the beneficial effect of the cell therapy may increase with more than one injection. To answer these important questions, the CCl₄ model of hepatic fibrosis (oral gavage 100 ul of 40% CCl₄ twice a week) can be used in addition to the CDAHFD model of NASH [19]. For all cell therapy experiments, 500,000 quiescent-like HSCs can be reprogrammed with miR-15a or miR-412. All appropriate controls such as groups with no treatment and treatment with HSCs that have not been reprogrammed can be included.

To test whether quiescent-like HSCs can prevent or reverse hepatic fibrosis, two separate experiments with different cell injection schedules can be used. All CCl₄ administrations can be done by oral gavage twice a week. Mice challenged with CCl₄ for 8 weeks normally develop advanced fibrosis. Reprogrammed HSCs can be injected on the second, fourth, and sixth week during the 8 week course of CCl₄ challenge. All mice can be sacrificed at the end of week 8. To test whether the cell therapy could reverse pre-existing hepatic fibrosis compared to control, reprogrammed HSCs can be injected on the ninth and eleventh week after 8 week course of CCl₄ challenge has been completed. All mice can be sacrificed at the end of week 12. The choline-deficient, L-amino acid defined, high fat diet (CDAHFD) model develops significant NASH by week 3 and stage 2 fibrosis (on a 0-4 scale) by week 9 [19]. A single injection of cell therapy on week 5 or three injections on weeks 3, 5, and 7 can be completed. All mice can be sacrificed at the end of week 9.

It has been shown previously that activated human HSCs can revert to a quiescent-like state after receiving human orthologue of miR-15a or miR-412 (FIG. 8). These reverted cells re-formed retinoid positive lipid droplets and were able to decrease pro-inflammatory cytokine expression from cocultured human hepatocellular carcinoma cell lines HepG2 and Huh7 (FIG. 6). As a bridge to translate the discovery for human therapies, a primary human HSC can be used for treating the CCL4 mouse model of hepatic fibrosis in determining the therapeutic potential of miR15a and miR-412. CCl₄ can be gavaged twice per week to induce the hepatic fibrosis in severe combined immune deficient mice (SCID) which were chosen to prevent immune rejection of human cells. CCL4-based hepatic fibrosis models have been generated successfully with SCID mice by other groups [37]. Fibrosis prevention study: human HSCs can be injected on second, forth, and sixth week during an 8 week course of CCL4 challenge in SCID mice. Fibrosis reversion study: To test whether reprogrammed human HSCs could reverse pre-existing hepatic fibrosis compared to control, HSCs can be injected on the ninth and eleventh week after 8 week course of 0014 challenge has been completed. All mice can be sacrificed at the end of week 12.

Initial experiments demonstrated that injecting HSCs reprogrammed with miR-15a or miR-412 can decrease collagen expression and the overall level of liver damage in CCl₄-induced hepatic fibrosis. One way to deliver the miRNA is to inject a lentivirus expressing miRNA. CCl₄ Fibrosis study: A lentivirus expressing either miR-15a or miR-412 can be injected into the tail vein on the second, fourth, and sixth week during the 8 week course of CCl₄ challenge. All mice can be sacrificed at the end of week 8. CDAHFD NASH study: A lentivirus expressing either miR-15a or miR-412 can be injected on weeks 3, 5, and 7 during 9 weeks of CDAHFD. All mice can be sacrificed at the end of week 9.

As another delivery method, the viability of injecting more stable, chemically modified miRNA mimics (Exiqon and Invitrogen) packaged in a lipid based carrier (MaxSuppressor™ by B100 Scientific) can be tested [38, 39]. By avoiding lentivirus, this method or its variation has the potential of being used in humans. CCl₄ Fibrosis study: Lipid carriers containing either miR-15a or miR-412 can be injected into the tail vein on the second, fourth, and sixth week during the 8 week course of CCl₄ challenge. All mice can be sacrificed at the end of week 8. CDAHFD NASH study: Lipid carriers containing either miR-15a or miR-412 can be injected on weeks 3, 5, and 7 during 9 weeks of CDAHFD. All mice can be sacrificed at the end of week 9.

### Defining the Function of WISP1 in Hepatic Steatosis, Inflammation, and Fibrosis.

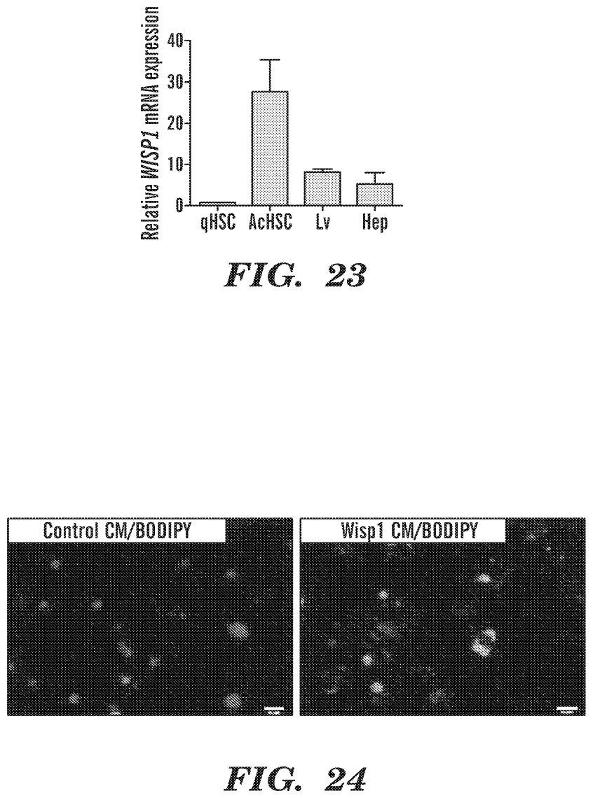

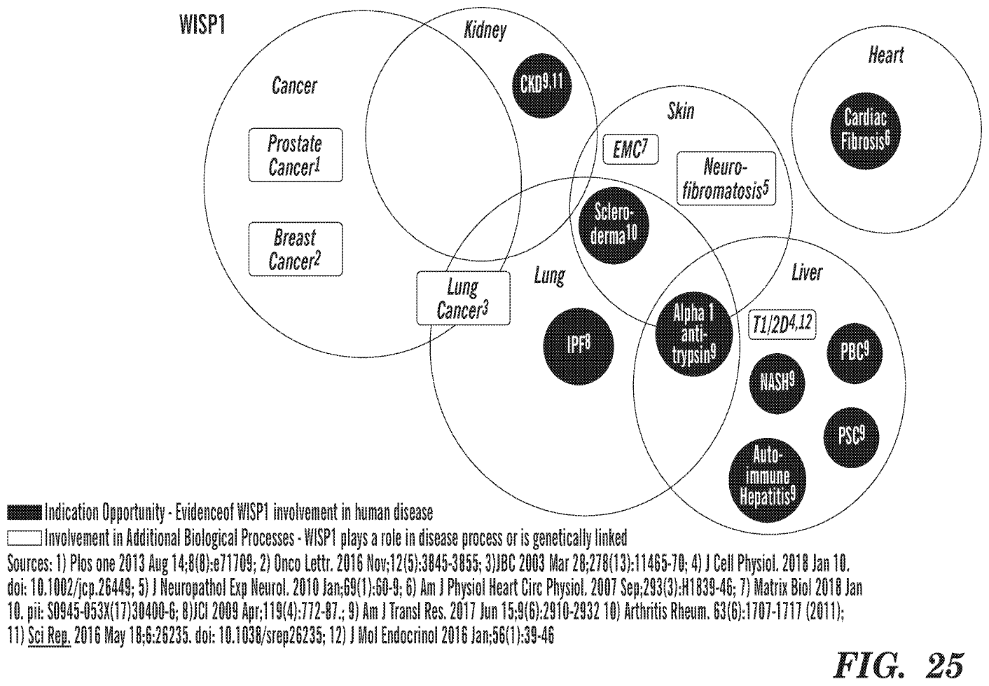

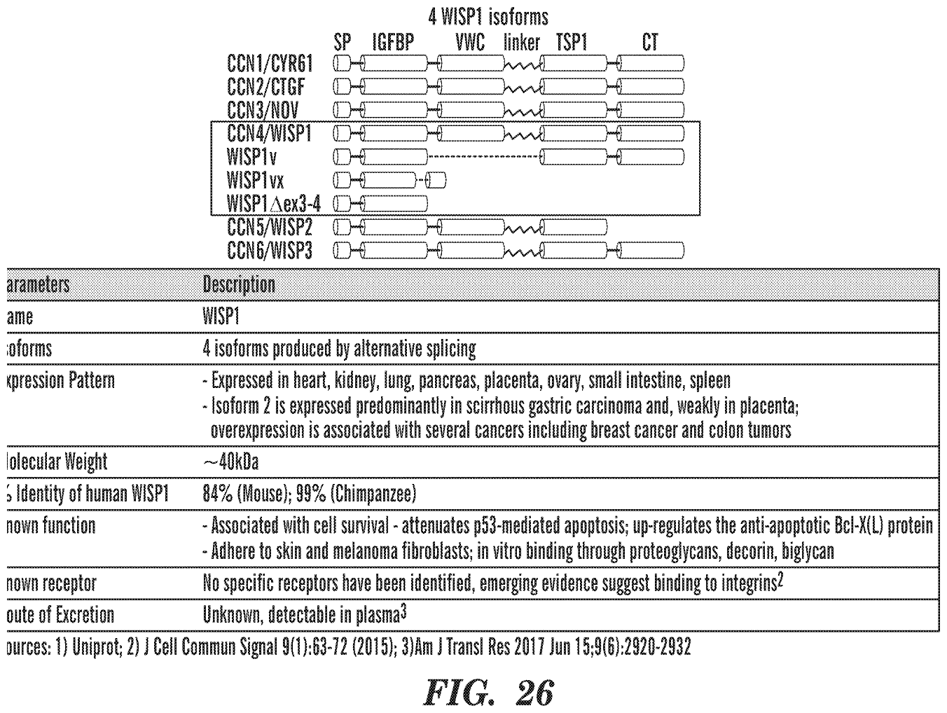

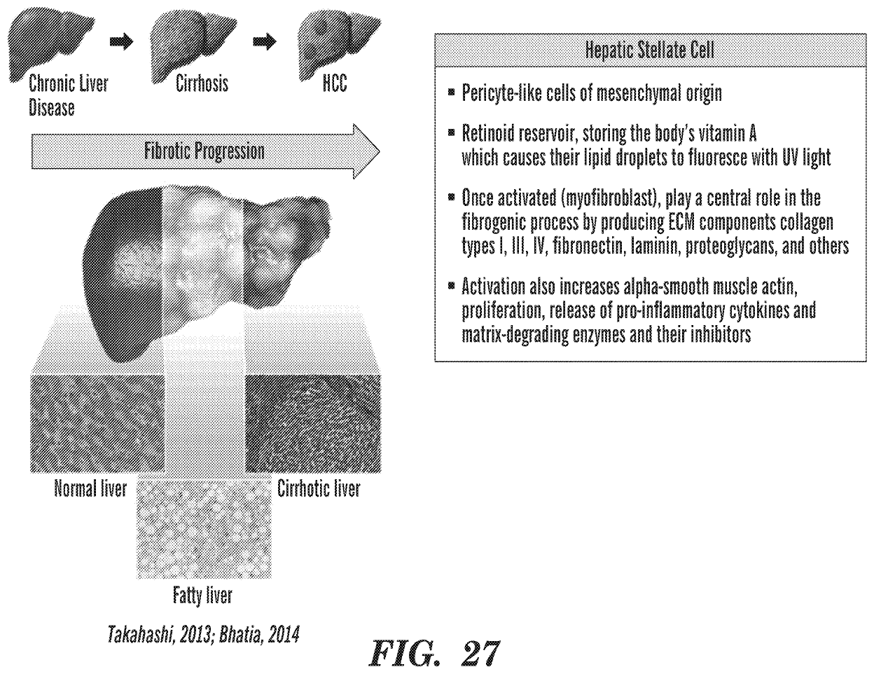

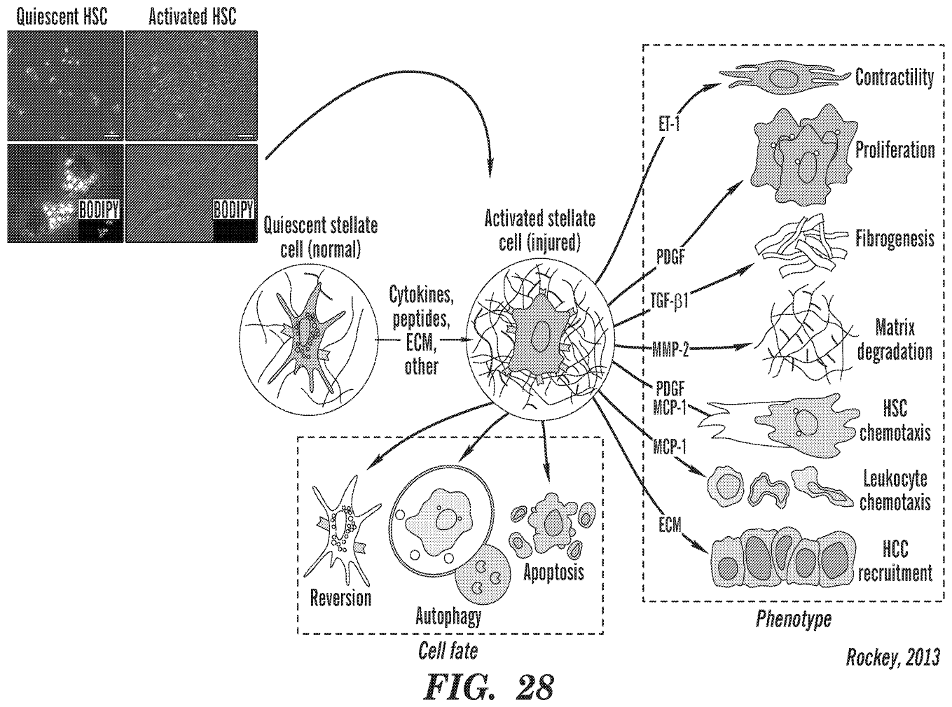

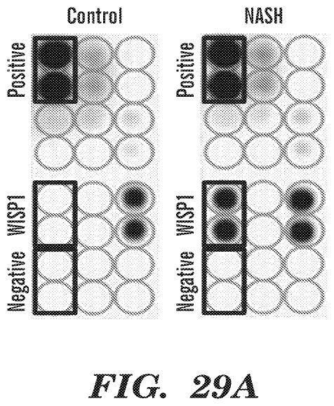

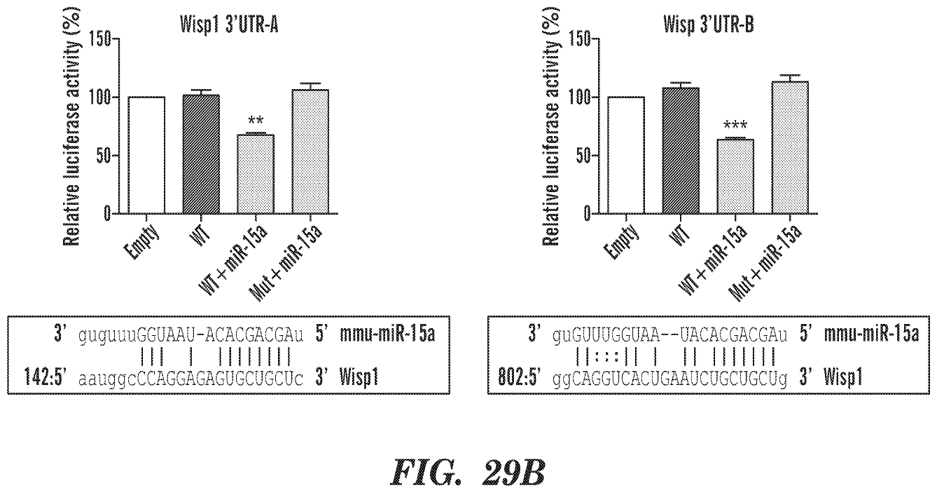

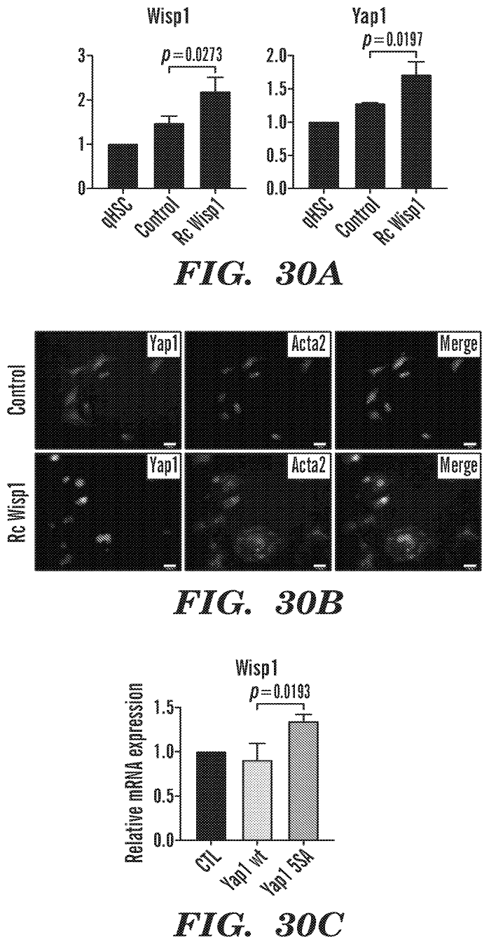

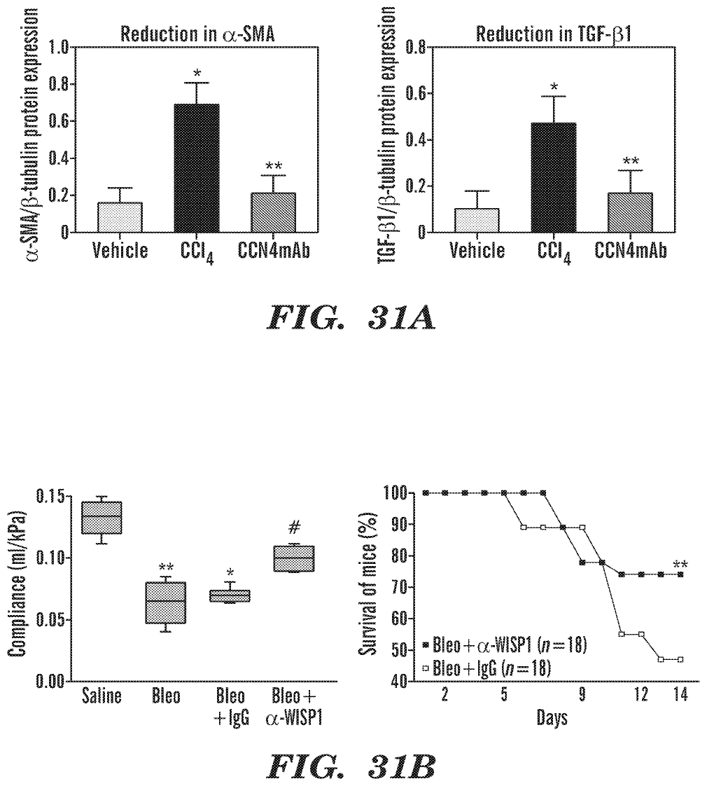

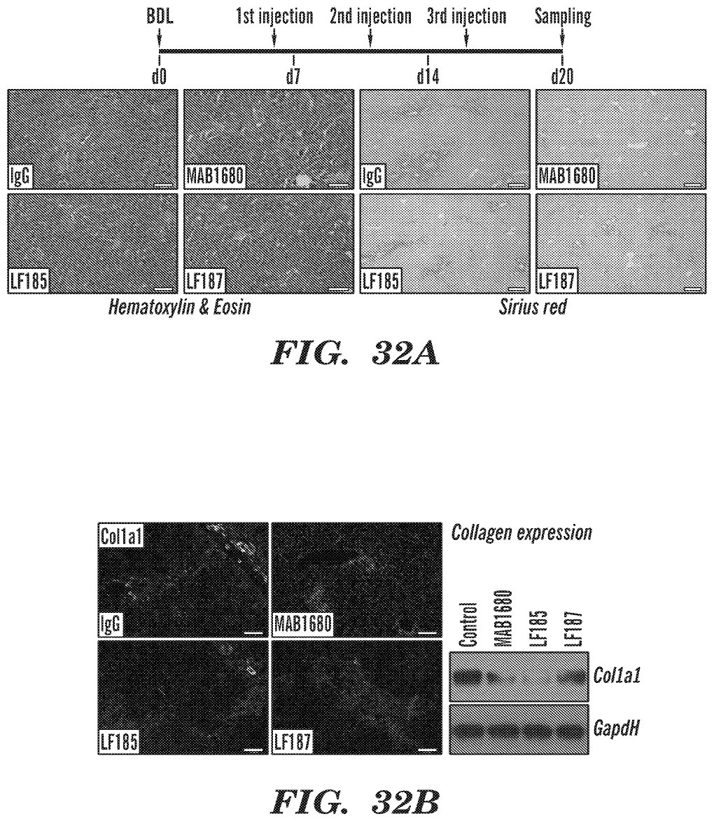

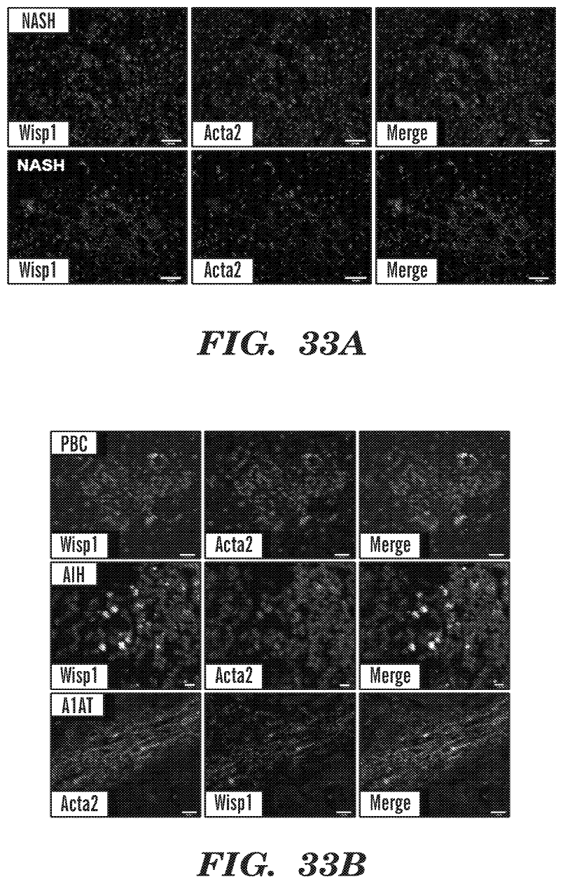

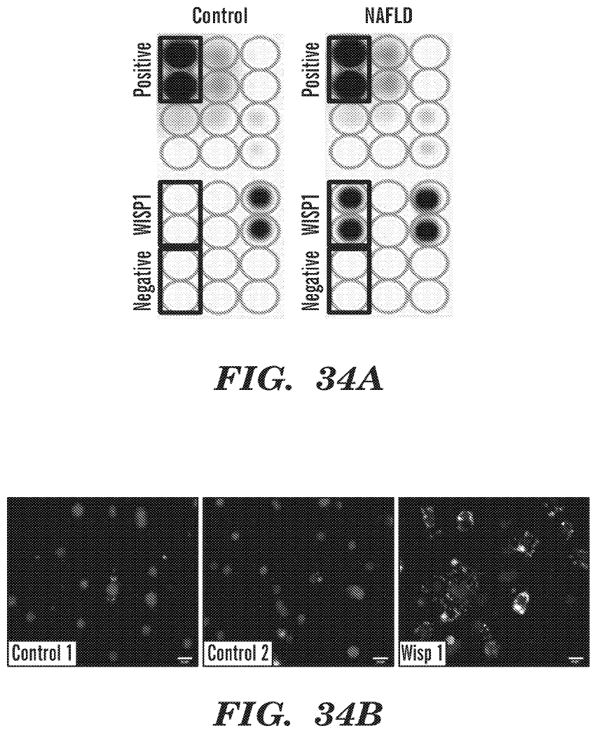

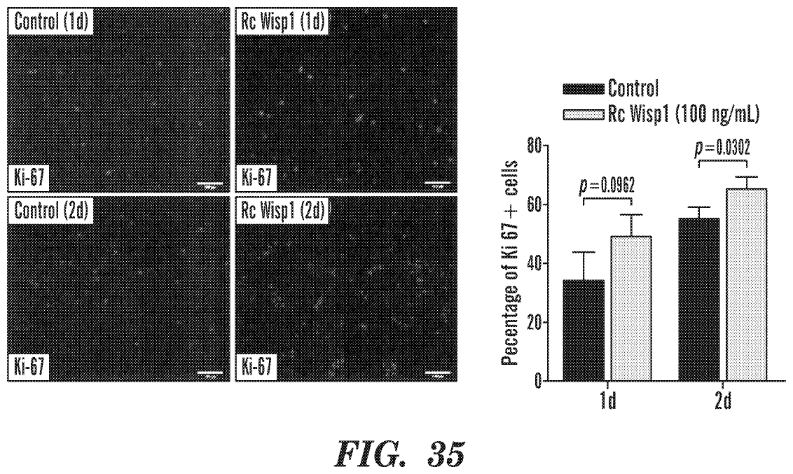

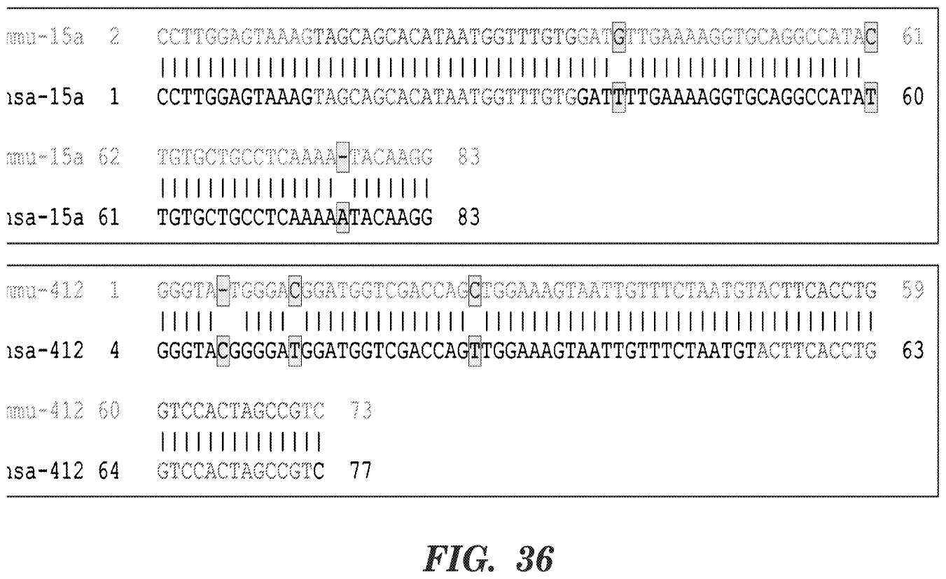

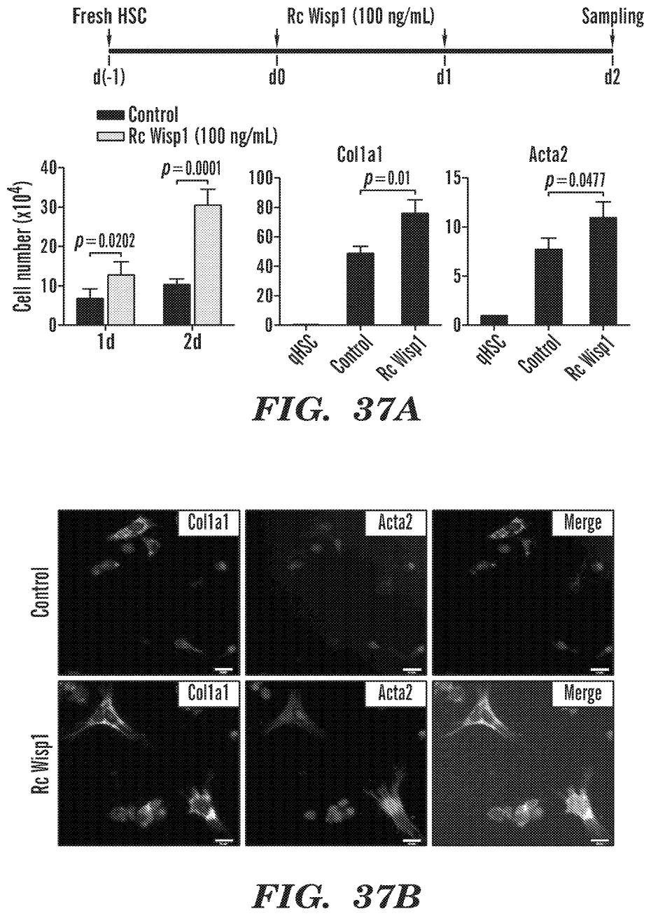

Knowing that miRNAs in general function by targeting multiple coding genes, these two candidates likely downregulate HSC genes that promote hepatic inflammation and fibrosis. Although there are probably many genes repressed by miR-15a and miR-412, WISP1 has emerged as the first experimentally validated direct target of miR-15a. WISP1 was first identified as one of several up-secreted proteins in the CDAHFD induced NASH-HSC conditioned media, detected using a cytokine array blot that holds >100 representative cytokines, chemokines, and extracellular matrix proteins (FIG. 22). More detailed analysis showed that activated HSCs expressed WISP1 almost 30 times the level of quiescent HSCs (FIG. 23). WISP1 is a member of the Ccn family of matricellular proteins that include Cyr61 (Ccn1), Ctgf (Ccn2), Nov (Ccn3), WISP1 (Ccn4), Wisp2 (Ccn5), and Wisp3 (Ccn6). Ctgf is already established as an important profibrotic factor in the liver [42]. Interestingly, WISP1 was found to be upregulated in human idiopathic pulmonary fibrosis, and treating bleomycin challenged mice with neutralizing WISP1 antibody caused attenuation of lung fibrosis [43]. Moreover, a small animal study showed that blocking WISP1 ameliorates CCl₄ induced liver fibrosis [44]. However, the role of WISP1 in the liver has not been researched in detail, especially in the context of NASH.

Knowing that CDAHFD-induced NASH-HSCs promote steatosis in cocultured hepatocytes (FIG. 14, 16), WISP1 can contribute to this phenotype. Indeed, when WISP1 was overexpressed in HSCs harvested from healthy mice, the conditioned media from these cells caused steatosis in initially healthy primary hepatocytes (FIG. 24). Furthermore, miR-15a is predicted to target WISP1 which contains two potential miR-15a binding sequences within its 3′-UTR. Thus, the putative target sequences or their mutated variants were cloned behind a constitutively active luciferase reporter gene. When co-transfected one of these reporters with miR-15a mimic, the reporters with wildtype sequences had decreased luciferase expression while the reporters with mutated sequences did not, indicating that miR-15a binds to both WISP1 target sequences (FIG. 11). Given these data and the published literature, WISP1 may be a key target gene for accomplishing some of the miR-15a actions in HSCs and may also be a drug target for managing NASH. It is contemplated herein that the hepatic function of WISP1 is a potential as a treatment target for NASH.

Although WISP1 is a direct target of miR-15a, its influence on HSC activation status and its role in NAFLD pathogenesis is unclear. A WISP1-null mouse line has been generated to demonstrate WISP1's role in bone formation, but its function in the liver was not studied [45]. These mice are fertile and do not have an overt hepatic phenotype. Their mutant allele is preserved in frozen sperm and embryos at an MMRRC facility in University of California at Davis who guarantees successful mutant mouse derivation (see attached letter). First the function of WISP1 can be elucidated in HSCs by harvesting them from WISP1-null mice and characterizing their phenotype ex vivo.

Wildtype HSCs in quiescent state become activated after 7-10 days of culturing on plastic, and RNA-seq data presented herein demonstrate a clear difference in global gene expression between these two groups (FIG. 11). A parallel experiment using WISP1-null HSCs can be used to assess whether WISP1 deletion changes the global expression profile toward that of HSC quiescence even in an activation-promoting environment. After isolating HSCs from WISP1-null mice, the cells can be cultured on plastic dish for one (quiescent) or 28 days (activated in wildtype). Their overall gene expression can be determined through RNA-seq and compare the results to those of wildtype HSCs. The global comparison can be done with multi-dimensional scaling analysis, and differentially expressed genes can be identified for detailed analysis.

Cell morphology, proliferation, and migration can be compared while cultured on plastic. Morphology can be determined using parameters such as size, extent of cell processes, existence of lipid droplets and whether they are UV-fluorescence positive, a sign of retinoid storage and quiescence. Proliferation can be checked with PCNA and Ki-67 immunostains in case WISP1-null HSCs have decreased proliferation, similar to quiescent HSCs. MTT assay can also be performed to quantify the cell proliferation rate as done in the past [32]. Since the preliminary data presented herein and the published literature point to WISP1 being a pro-fibrotic gene, deleting WISP1 may promote quiescence in HSCs, perhaps even while being cultured on plastic dish. Finally, reconstituting WISP1 in the knockout HSCs can rescue the wild-type phenotype. The reconstituting WISP1 can be achieved by transfecting a WISP1-expressing piggyBac vector. WISP1-null HSCs that were transfected with the WISP1-expressing vector can be characterized with the same in vitro assays that were used on WISP1-null cells that were not transfected.

WISP1-null HSCs' interaction with hepatocytes can be evaluated with the coculture system described herein. Activated wildtype HSCs cause cocultured hepatocytes to become steatotic (FIG. 14). HSCs overexpressing WISP1 induce nearby hepatocytes to become steatotic by even greater severity (FIG. 24). The WISP1-null HSCs can have an attenuated pro-steatotic phenotype. The readout of the coculture assay includes Bodipy stain for lipid droplets and PCR of cocultured hepatocytes to measure the expression of pro-inflammatory factors.

The phenotype can be assessed by the overall severity of hepatic pathology. WISP1-null mice can be challenged with CCl₄ and CDAHFD to model toxin induced hepatic fibrosis and NASH, respectively. The resulting liver damage sustained by WISP1-null mice can be assessed with that of wildtype mice. Given the known profibrotic role of WISP1 in other organs including the lung, it is expected that WISP1-null mice have a decreased hepatic fibrosis and perhaps even decreased steatosis and inflammation. For all in vivo experiments, the liver phenotype can be assessed by performing PCR of profibrotic genes in the liver, histology including H&E and Sirius Red stain, hepatic hydroxyproline assay, and measuring plasma ALT. Lastly, the functional phenotype of HSCs harvested from these mouse models of liver disease can be determined.

CCl₄ Fibrosis model: Male wildtype and WISP1-null mice can be challenged with twice weekly CCl₄ gavage. They can receive CCl₄ for 8 weeks and can be sacrificed at the end of week 8. CDAHFD NASH model: CDAHFD to wildtype and WISP1-null mice can be fed for 9 weeks. Half of the mice can be sacrificed at the end of week 6 and the other half at the end of week 9 to define the role of WISP1 in moderate NASH (6 weeks of CDAHFD) and late-stage NASH with stage 2 fibrosis (9 weeks of CDAHFD).

HSCs can be isolated from mice challenged with 4 weeks of CCl₄ and 3 weeks of CDAHFD. These HSCs can be cocultured with healthy hepatocytes to determine whether they develop steatosis and express pro-inflammatory factors. The results can be compared to those of wildtype HSCs that underwent the same challenge.

Blocking WISP1 with a neutralizing antibody attenuated the bleomycin-induced damage in the mouse lung [43]. Similarly, neutralizing WISP1 may decrease the level of inflammation and fibrosis that occur in NASH. A mouse model of NASH can be treated with WISP1 antibodies and assess the liver phenotype after 6 and 9 weeks of therapy.

CDAHFD NASH model: CDAHFD to wildtype mice can be fed for 9 weeks. The treatment group can receive either the commercially available antibody (R&D Systems) [43] or the antibody by weekly injection [46]. The control group can receive IgG. Half of the mice can be sacrificed at the end of week 6 and the other half at the end of week 9 to test the effect of WISP1 blockage in moderate and late-stage NASH with fibrosis respectively.

Further, analyzing the conditioned media of HSCs from NASH, secreted paracrine or autocrine factors can be identified. A more detailed analysis of the NASH-HSC conditioned media through mass spectrometry could identify other mediators that may be drug targets. Finally, the coculture system described herein could be used to study other liver diseases as long as there are representative mouse models. For example, the interaction of hepatocytes and HSCs can be modeled in alcoholic liver disease by isolating cell types in alcohol-fed mice [47].

### REFERENCES—EXAMPLE 1/2

1. Clark J M, Brancati F L and Diehl A M (2003) The prevalence and etiology of elevated aminotransferase levels in the United States. Am J Gastroenterol 98:960-7. doi: 10.1111/0572-0241.2003.07486.x
2. Charlton M R, Burns J M, Pedersen R A, Watt K D, Heimbach J K and Dierkhising R A (2011) Frequency and outcomes of liver transplantation for nonalcoholic steatohepatitis in the United States. Gastroenterology 141:1249-53. doi: 10.1053/j.gastro.2011.06.061
3. Starley B Q, Calcagno C J and Harrison S A (2010) Nonalcoholic fatty liver disease and hepatocellular carcinoma: a weighty connection. Hepatology 51:1820-32. doi: 10.1002/hep.23594
4. Younossi Z M, Blissett D, Blissett R, Henry L, Stepanova M, Younossi Y, Racila A, Hunt S and Beckerman R (2016) The economic and clinical burden of nonalcoholic fatty liver disease in the United States and Europe. Hepatology 64:1577-1586. doi: 10.1002/hep.28785
5. Lassailly G, Caiazzo R, Pattou F and Mathurin P (2016) Perspectives on Treatment for Nonalcoholic Steatohepatitis. Gastroenterology. doi: 10.1053/j.gastro.2016.03.004
6. Friedman S L (2008) Mechanisms of hepatic fibrogenesis. Gastroenterology 134:1655-69. doi: 10.1053/j.gastro.2008.03.003
7. Friedman S L (2008) Hepatic stellate cells: protean, multifunctional, and enigmatic cells of the liver. Physiol Rev 88:125-72. doi: 10.1152/physrev.00013.2007
8. Puche J E, Lee Y A, Jiao J, Aloman C, Fiel M I, Munoz U, Kraus T, Lee T, Yee H F, Jr. and Friedman S L (2013) A novel murine model to deplete hepatic stellate cells uncovers their role in amplifying liver damage in mice. Hepatology 57:339-50. doi: 10.1002/hep.26053
9. Kisseleva T, Cong M, Paik Y, Scholten D, Jiang C, Benner C, lwaisako K, Moore-Morris T, Scott B, Tsukamoto H, Evans S M, Dillmann W, Glass C K and Brenner D A (2012) Myofibroblasts revert to an inactive phenotype during regression of liver fibrosis. Proc Natl Acad Sci USA 109:9448-53. doi: 10.1073/pnas.1201840109
10. Mederacke I, Hsu C C, Troeger J S, Huebener P, Mu X, Dapito D H, Pradere J P and Schwabe R F (2013) Fate tracing reveals hepatic stellate cells as dominant contributors to liver fibrosis independent of its aetiology. Nat Commun 4:2823. doi: 10.1038/ncomms3823
11. Guo C J, Pan Q, Cheng T, Jiang B, Chen G Y and Li D G (2009) Changes in microRNAs associated with hepatic stellate cell activation status identify signaling pathways. FEBS J 276:5163-76. doi: 10.1111/0742-4658.2009.07213.x
12. Maubach G, Lim M C, Chen J, Yang H and Zhuo L (2011) miRNA studies in in vitro and in vivo activated hepatic stellate cells. World J Gastroenterol 17:2748-73. doi: 10.3748/wjg.v17.i22.
13. Lakner A M, Steuerwald N M, Walling T L, Ghosh S, Li T, McKillop I H, Russo M W, Bonkovsky H L and Schrum L W (2012) Inhibitory effects of microRNA 19b in hepatic stellate cell-mediated fibrogenesis. Hepatology 56:300-10. doi: 10.1002/hep.25613
14. Schuppan D and Kim Y O (2013) Evolving therapies for liver fibrosis. J Clin Invest 123:1887-901. doi: 10.1172/JCI66028
15. Corey K E and Rinella M E (2016) Medical and Surgical Treatment Options for Nonalcoholic Steatohepatitis. Dig Dis Sci. doi: 10.1007/s10620-016-4083-8
16. Ivey K N and Srivastava D (2010) MicroRNAs as regulators of differentiation and cell fate decisions. Cell Stem Cell 7:36-41.
17. Griffin T A, Anderson H C and Wolfe J H (2015) Ex vivo gene therapy using patient iPSC-derived NSCs reverses pathology in the brain of a homologous mouse model. Stem Cell Reports 4:835-46. doi: 10.1016/j.stemcr.2015.02.022
18. Terai S and Tsuchiya A (2017) Status of and candidates for cell therapy in liver cirrhosis: overcoming the “point of no return” in advanced liver cirrhosis. J Gastroenterol 52:129-140. doi: 10.1007/s00535-016-1258-1
19. Matsumoto M, Hada N, Sakamaki Y, Uno A, Shiga T, Tanaka C, Ito T, Katsume A and Sudoh M (2013) An improved mouse model that rapidly develops fibrosis in non-alcoholic steatohepatitis. Int J Exp Pathol 94:93-103. doi: 10.1111/iep.12008
20. Herrmann J, Gressner A M and Weiskirchen R (2007) Immortal hepatic stellate cell lines: useful tools to study hepatic stellate cell biology and function?J Cell Mol Med 11:704-22. doi: 10.1111/j.1582-4934.2007.00060.x
21. Mederacke I, Dapito D H, Affo S, Uchinami H and Schwabe R F (2015) High-yield and high-purity isolation of hepatic stellate cells from normal and fibrotic mouse livers. Nat Protoc 10:305-15. doi: 10.1038/nprot.2015.017
22. Younossi Z M, Koenig A B, Abdelatif D, Fazel Y, Henry L and Wymer M (2015) Global Epidemiology of Non-Alcoholic Fatty Liver Disease-Meta-Analytic Assessment of Prevalence, Incidence and Outcomes. Hepatology. doi: 10.1002/hep.28431
23. Geerts A (2001) History, heterogeneity, developmental biology, and functions of quiescent hepatic stellate cells. Semin Liver Dis 21:311-35. doi: 10.1055/s-2001-17550
24. Yin C, Evason K J, Asahina K and Stainier D Y (2013) Hepatic stellate cells in liver development, regeneration, and cancer. J Clin Invest 123:1902-10. doi: 10.1172/J0166369
25. Deleve L D, Wang X and Guo Y (2008) Sinusoidal endothelial cells prevent rat stellate cell activation and promote reversion to quiescence. Hepatology 48:920-30. doi: 10.1002/hep.22351
26. Tijsen A J, van der Made I, van den Hoogenhof M M, Wijnen W J, van Deel E D, de Groot N E, Alekseev S, Fluiter K, Schroen B, Goumans M J, van der Velden J, Duncker D J, Pinto Y M and Creemers E E (2014) The microRNA-15 family inhibits the TGFbeta-pathway in the heart. Cardiovasc Res 104:61-71. doi: 10.1093/cvr/cvu184
27. Ageilan R I, Calin G A and Croce C M (2010) miR-15a and miR-16-1 in cancer: discovery, function and future perspectives. Cell Death Differ 17:215-20. doi: 10.1038/cdd.2009.69
28. Klein U, Lia M, Crespo M, Siegel R, Shen Q, Mo T, Ambesi-Impiombato A, Califano A, Migliazza A, Bhagat G and Dalla-Favera R (2010) The DLEU2/miR-15a/16-1 cluster controls B cell proliferation and its deletion leads to chronic lymphocytic leukemia. Cancer Cell 17:28-40. doi: 10.1016/j.ccr.2009.11.019
29. Szabo G and Csak T (2016) Role of MicroRNAs in NAFLD/NASH. Dig Dis Sci. doi: 10.1007/s10620-015-4002-4
30. Kim B M, Thier M C, Oh S, Sherwood R, Kanellopoulou C, Edenhofer F and Choi M Y (2012) MicroRNAs are indispensable for reprogramming mouse embryonic fibroblasts into induced stem cell-like cells. PLoS One 7:e39239.
31. Kanellopoulou C, Muljo S A, Kung A L, Ganesan S, Drapkin R, Jenuwein T, Livingston D M and Rajewsky K (2005) Dicer-deficient mouse embryonic stem cells are defective in differentiation and centromeric silencing. Genes Dev 19:489-501.
32. Kim B M and Choi M Y (2012) Non-canonical microRNAs miR-320 and miR-702 promote proliferation in Dgcr8-deficient embryonic stem cells. Biochem Biophys Res Commun 426:183-9.
33. Betel D, Koppal A, Agius P, Sander C and Leslie C (2010) Comprehensive modeling of microRNA targets predicts functional non-conserved and non-canonical sites. Genome Biol 11:R90.
34. Mi H, Muruganujan A and Thomas P D (2013) PANTHER in 2013: modeling the evolution of gene function, and other gene attributes, in the context of phylogenetic trees. Nucleic Acids Res 41:D377-86. doi: 10.1093/nar/gks1118
35. Daneshvar K, Pondick J V, Kim B M, Zhou C, York S R, Macklin J A, Abualteen A, Tan B, Sigova A A, Marcho C, Tremblay K D, Mager J, Choi M Y and Mullen A C (2016) DIGIT Is a Conserved Long Noncoding RNA that Regulates GSC Expression to Control Definitive Endoderm Differentiation of Embryonic Stem Cells. Cell Rep 17:353-365. doi: 10.1016/j.celrep.2016.09.017
36. Sohara N, Znoyko I, Levy M T, Trojanowska M and Reuben A (2002) Reversal of activation of human myofibroblast-like cells by culture on a basement membrane-like substrate. J Hepatol 37:214-21.
37. Melhem A, Muhanna N, Bishara A, Alvarez C E, Ilan Y, Bishara T, Horani A, Nassar M, Friedman S L and Safadi R (2006) Anti-fibrotic activity of N K cells in experimental liver injury through killing of activated HSC. J Hepatol 45:60-71. doi: 10.1016/j.jhep.2005.12.025
38. Trang P, Wiggins J F, Daige C L, Cho C, Omotola M, Brown D, Weidhaas J B, Bader A G and Slack F J (2011) Systemic delivery of tumor suppressor microRNA mimics using a neutral lipid emulsion inhibits lung tumors in mice. Mol Ther 19:1116-22. doi: 10.1038/mt.2011.48
39. Wiggins J F, Ruffino L, Kelnar K, Omotola M, Patrawala L, Brown D and Bader A G (2010) Development of a lung cancer therapeutic based on the tumor suppressor microRNA-34. Cancer Res 70:5923-30. doi: 10.1158/0008-5472.CAN-10-0655
40. Song G, Pacher M, Balakrishnan A, Yuan Q, Tsay H C, Yang D, Reetz J, Brandes S, Dai Z, Putzer B M, Arauzo-Bravo M J, Steinemann D, Luedde T, Schwabe R F, Manns M P, Scholer H R, Schambach A, Cantz T, Ott M and Sharma A D (2016) Direct Reprogramming of Hepatic Myofibroblasts into Hepatocytes In Vivo Attenuates Liver Fibrosis. Cell Stem Cell 18:797-808. doi: 10.1016/j.stem.2016.01.010
41. Rezvani M, Espanol-Suner R, Malato Y, Dumont L, Grimm A A, Kienle E, Bindman J G, Wiedtke E, Hsu B Y, Naqvi S J, Schwabe R F, Corvera C U, Grimm D and Willenbring H (2016) In Vivo Hepatic Reprogramming of Myofibroblasts with AAV Vectors as a Therapeutic Strategy for Liver Fibrosis. Cell Stem Cell 18:809-16. doi: 10.1016/j.stem.2016.05.005
42. Chen L, Charrier A, Zhou Y, Chen R, Yu B, Agarwal K, Tsukamoto H, Lee L J, Paulaitis M E and Brigstock D R (2014) Epigenetic regulation of connective tissue growth factor by MicroRNA-214 delivery in exosomes from mouse or human hepatic stellate cells. Hepatology 59:1118-29. doi: 10.1002/hep.26768
43. Konigshoff M, Kramer M, Balsara N, Wilhelm J, Amarie O V, Jahn A, Rose F, Fink L, Seeger W, Schaefer L, Gunther A and Eickelberg 0 (2009) WNT1-inducible signaling protein-1 mediates pulmonary fibrosis in mice and is upregulated in humans with idiopathic pulmonary fibrosis. J Clin Invest 119:772-87. doi: 10.1172/JC133950
44. Li X, Chen Y, Ye W, Tao X, Zhu J, Wu S and Lou L (2015) Blockade of CCN4 attenuates CCl₄-induced liver fibrosis. Arch Med Sci 11:647-53. doi: 10.5114/aoms.2015.52371
45. Maeda A, Ono M, Holmbeck K, Li L, Kilts™, Kram V, Noonan M L, Yoshioka Y, McNerny E M, Tantillo M A, Kohn D H, Lyons K M, Robey P G and Young M F (2015) WNT1-induced Secreted Protein-1 (WISP1), a Novel Regulator of Bone Turnover and Wnt Signaling. J Biol Chem 290:14004-18. doi: 10.1074/jbc.M114.628818
46. Ono M, lnkson C A, Sonn R, Kilts™, de Castro L F, Maeda A, Fisher L W, Robey P G, Berendsen A D, Li L, McCartney-Francis N, Brown A C, Crawford N P, Molinolo A, Jain A, Fedarko N S and Young M F (2013) WISP1/CCN4: a potential target for inhibiting prostate cancer growth and spread to bone. PLoS One 8:e71709. doi: 10.1371/journal.pone.0071709
47. Hamarneh S R, Kim B M, Kaliannan K, Morrison S A, Tantillo T J, Tao Q, Mohamed MMR, Ramirez J M, Karas A, Liu W, Hu D, Teshager A, Gul S S, Economopoulos K P, Bhan A K, Maio M S, Choi M Y and Hodin R A (2017) Intestinal Alkaline Phosphatase Attenuates Alcohol-Induced Hepatosteatosis in Mice. Dig Dis Sci. doi: 10.1007/s10620-017-4576-0

### Example 3—HSC Isolation Protocol

The following protocol is used in experiments described herein to isolate HSCs, e.g., mod-Hep/HSC.
1. Canulate the portal vein with the help of an angiocath or butterfly needle.
2. Perfuse the liver with solutions at 37° C. at 6 mL/min: 30 mL HBSS (without Ca²⁺ and Mg²⁺), shortly after beginning perfusion, cut the IVC 30 mL 0.05% collagenase [0.5 mg/mL->for 50 mL, 500 ul of 50 mg/mL Collagenase stock] (e.g., Collagenase B; Roche 11088815001).
3. After perfusion, excise the liver carefully (especially gall bladder) and remove the Glisson's capsule.
4. Transfer the liver into a sterile beaker and cut into VERY thin pieces.
5. Add the remaining 20 mL of 0.05% collagenase and keep in incubator at 37° C. for 30 min [add DNAse (10 ug/mL)] (e. g., DNase I; Roche 10104159001).
6. Pipette up and down and filter with a Cell Strainer (70 um) in a Petri dish. Add PBS+DNAse (10 ug/mL) up to 45 mL.
7. Remove hepatotocyte by centrifugation at ×50 g for 3 min. and transfer supernatant to new tube.
8. Pellet by centrifugation at ×635 g for 10 min.
9. Wash with 45 mL PBS+DNAse (10 ug/mL) and centrifuge at ×635 g for 10 min.
10. Filter the cell suspension through a 70 um cell strainer.
11. Wash with 45 mL PBS+DNAse (10 ug/mL) and centrifuge at ×635 g for 10 min.
12. Aspirate supernatant. Add PBS to the pellet to a total volume of 6.5 mL.
13. Add 3.5 mL percoll (9 parts Percoll, 1 part 10×PBS) to a total volume of 10 mL, mix, and transfer to a
15 mL tube (e.g., Percoll; Percoll plus, GE Healthcare 17-5445-02).
14. Carefully add 1 mL of PBS to the top of the column.
15. Centrifuge the columns at RT, 30 min, ×1130 g) (acceleration 9, brake 0).
16. After centrifugation, HSCs are the in the layer located between the PBS and 35% percoll.
17. Aspirate HSCs with a micropipette and wash with 10 mL DMEM and centrifuge at ×635 g×6 min.
18. Aspirate supernatant.
19. Resuspend pellet and plate in DMEM+20% FBS+1% P/S.

### Example 4: Antibodies that Inhibit Wisp1 for the Treatment and Prevention of Liver Diseases

Antibodies that specifically bind and inhibit WISP1 can be used to treat and prevent liver diseases such as primary biliary cholangitis (PBC) and autoimmune hepatitis (AIH).

Specifically, lead candidates that inhibit WISP1 include ant-WISP1 IgG1/IgG4 antibodies and IgG1 ADCC). Strategies employed to engineer an anti-WISP1 antibody include but are not limited to Fe engineering for half-life extension; bi-specific antibody technologies to enable inhibition of two anti-fibrotic targets simultaneously; and sweeping technologies that (1) turn antibodies from sequestering entities into catalytic drugs by driving target catabolism, (2) dramatically decrease CoG, dosing levels, and frequency of dosing, (3) increases the likelihood of subcutaneous dosing, and (4) obviates the need for full blocking potential.

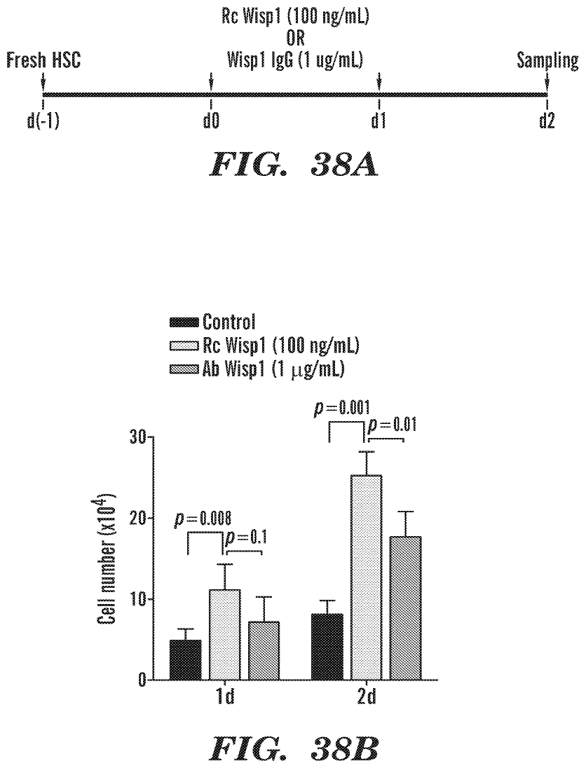

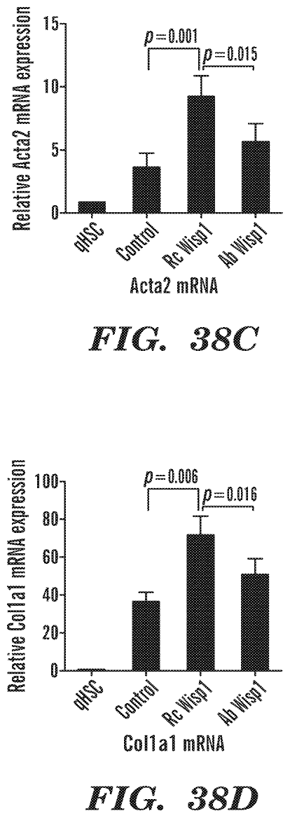

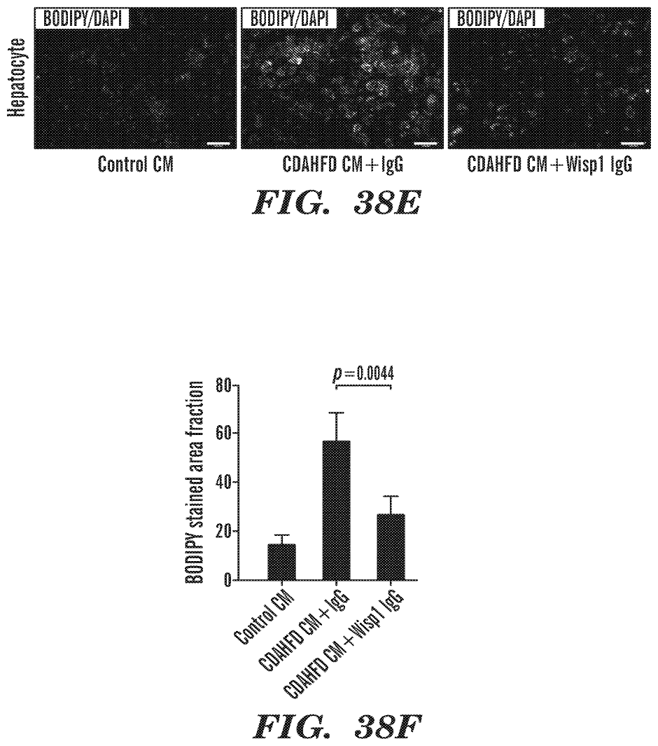

To assess the capacity of an anti-WISP1 antibody to inhibit WISP1, the antibody was tested on fresh HSCs. A WISP1 antibody (Ab WISP1 or WISP IgG, RND Systems, 1pg/mL) was incubated with conditioned media for 1 hour at 37° C. and was then applied to HSCs. As a negative control, the HSCs were incubated with only conditioned media. As a positive control, recombinant WISP1 (Re-WISP1, 100 ng/mL) was also added to the HSCs instead of the antibody. HSC samples were evaluated 48-hours post-treatment (FIG. 38A). Re-WISP1 is, e.g., WISP1-CCN4, obtained from the cell line identified as Accession #O54774, and is commercially available from R&D Systems. Re-WISP1 was found to significantly increased cell number at day 1 and day 2 post-incubation (FIG. 38B), Acta2 mRNA expression (FIG. 38C), and Colla1 mRNA expression (FIG. 38D) in HSCs, indicating that HSCs become activated in the presence of recombinant WISP1. Surprisingly, commercially available anti-WISP1 IgG (Ab Wisp1) prevented HSC activation in HSCs; incubation with anti-WISP1 resulted in significantly reduced cell numbers at day 1 and day 2 post-incubation (FIG. 38B), Acta2 mRNA expression (FIG. 38C), and Colla1 mRNA expression (FIG. 38D) as compared to HSCs incubated with rc WISP1.

Anti-WISP1 IgG was then tested on primary hepatocytes isolated from choline deficient, L-amino acid defined, high fat diet-induced steatosis (CDAHFD) mouse model. A hallmark of a liver disease, e.g., NALFD and viral hepatitis is an increase in lipid droplets in the diseased liver cells. To assess the presence of lipid droplets, BODIPY staining is employed. As expected, CDAHFD cells treated with control IgG have increased BODIPY staining (FIGS. 38E and 38F). In contrast, CDAHFD cells treated with anti-WISP1 IgG have decreased BODIPY staining, similar to a control, healthy cell (FIGS. 38E and 38F). Thus, data presented herein show that anti-WISP1 IgG can revert a diseased liver cell to a healthy liver cell.

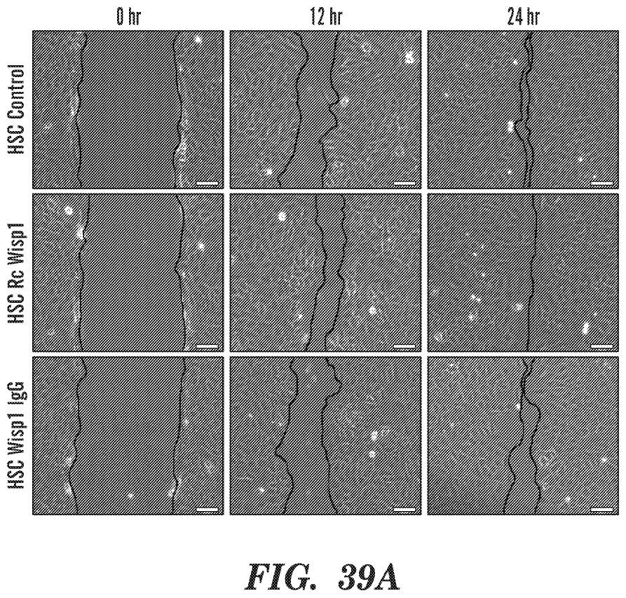

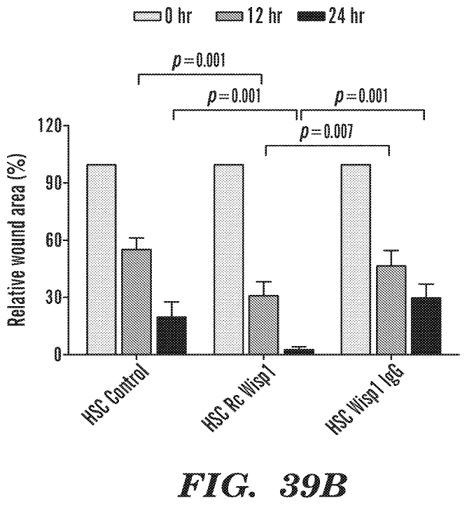

Finally, to show the capacity for an anti-WISP1 IgG to modulate HSC migration, HSCs were cultured to form a monolayer, and then injured (FIG. 39A-FIG. 39B). Cells were cultured in a 60-mm dish at 90% confluence in growth medium. A single linear scratch wound was generated using a sterile P200 pipette tip. The cells were washed with PBS to remove floating cellular debris and re-fed with recombinant Wisp1 protein (100 ng/mL, R&D Systems, Minneapolis, MN) and monoclonal Wisp1 antibody (1_g/mL, R&D Systems, Minneapolis, MN) for 24 h. Wound closure or cell migration was photographed when the scrape wound was introduced, 12, and 24 h after injury using an inverted microscope. The area between wound edges in dish at each time was measured using a standard template placed on the image. Data were expressed as wound area (percentage) relative to initial wound area. The wounded area was determined using publically available ImageJ (NIH) software.

Following injury, HSCs were incubated with either Rc WISP1 or anti-WISP1 IgG. HSCs incubated with rc WISP1 showed a more rapid migration as compared to control or anti-WISP1 IgG treated HSCs, particularly after the 12 hours time point. In contrast, HSCs incubated with anti-WISP1 IgG resulted in a slower migration, similar to a control treated HSC (FIG. 39A-FIG. 39B). Anti-WISP1 IgG prevented HSC migration following injury as compared to HSCs treated with Re-WISP1. This result confirms that antibodies that inhibit WISP1 can prevent HSC activation and migration, making them a useful agent to treat liver diseases.

SEQUENCES
SEQ ID NO: 1 is an amino acid sequence that encodes human WNT1-
inducible-signaling pathway protein 1 isoform 1 precursor.
MRWFLPWTLAAVTAAAASTVLATALSPAPTTMDFTPAPLEDTSSRPQFCKWPCECPPSPPRCPLGVSLIT
DGCECCKMCAQQLGDNCTEAAICDPHRGLYCDYSGDRPRYAIGVCAQVVGVGCVLDGVRYNNGQSFQ
PNC
KYNCTCIDGAVGCTPLCLRVRPPRLWCPHPRRVSIPGHCCEQWVCEDDAKRPRKTAPRDTGAFDAVGE
VE
AWHRNCIAYTSPWSPCSTSCGLGVSTRISNVNAQCWPEQESRLCNLRPCDVDIHTLIKAGKKCLAVYQPE
ASMNFTLAGCISTRSYQPKYCGVCMDNRCCIPYKSKTIDVSFQCPDGLGFSRQVLWINACFCNLSCRNPN
DIFADLESYPDFSEIAN
SEQ ID NO: 2 is an amino acid sequence that encodes human WNT1-
inducible-signaling pathway protein 1 isoform 2 precursor.
MRWFLPWTLAAVTAAAASTVLATALSPAPTTMDFTPAPLEDTSSRPQFCKWPCECPPSPPRCPLGVSLIT
DGCECCKMCAQQLGDNCTEAAICDPHRGLYCDYSGDRPRYAIGVCAHAVGEVEAWHRNCIAYTSPWSP
CSTSCGLGVSTRISNVNAQCWPEQESRLCNLRPCDVDIHTLIKAGKKCLAVYQPEASMNFTLAGCISTRS
YQ
PKYCGVCMDNRCCIPYKSKTIDVSFQCPDGLGFSRQVLWINACFCNLSCRNPNDIFADLESYPDFSEIAN
SEQ ID NO: 3 is an amino acid sequence that encodes human WNT1-
inducible-signaling pathway protein 1 isoform 3 precursor.
MRWFLPWTLAAVTAAAASTVLATALSPAPTTMDFTPAPLEDTSSRPQFCKWPCECPPSPPRCPLGVSLIT
DGCECCKMCAQQLGDNCTEAAICDPHRGLYCDYSGDRPRYAIGVCARREEVSGCVPARGIHELHTCGL
HQ
HTLLSTQVLWSLHGQ
SEQ ID NO: 4 is an amino acid sequence that encodes human WNT1-
inducible-signaling pathway protein 1 isoform 4 precursor.
MRWFLPWTLAAVTAAAASTVLATAGKKCLAVYQPEASMNFTLAGCISTRSYQPKYCGVCMDNRCCIPY
KSKTIDVSFQCPDGLGFSRQVLWINACFCNLSCRNPNDIFADLESYPDFSEIAN
SEQ ID NO: 5 is a nucleotide sequence of human WNT1 inducible
signaling pathway protein 1 (WISP1), transcript variant 1, mRNA.
ATATCTGGTGCTCCTGATGGGCCGGCCAGTCTGGGCCCAGCTCCCCCGAGAGGTGGTCGGATCCTCT
GGGCTGCTCGGTCGATGCCTGTGCCACTGACGTCCAGGCATGAGGTGGTTCCTGCCCTGGACGCTGG
CAGCAGTGACAGCAGCAGCCGCCAGCACCGTCCTGGCCACGGCCCTCTCTCCAGCCCCTACGACCAT
GGACTTTACCCCAGCTCCACTGGAGGACACCTCCTCACGCCCCCAATTCTGCAAGTGGCCATGTGAG
TGCCCGCCATCCCCACCCCGCTGCCCGCTGGGGGTCAGCCTCATCACAGATGGCTGTGAGTGCTGTA
AGATGTGCGCTCAGCAGCTTGGGGACAACTGCACGGAGGCTGCCATCTGTGACCCCCACCGGGGCC
TCTACTGTGACTACAGCGGGGACCGCCCGAGGTACGCAATAGGAGTGTGTGCACAGGTGGTCGGTG
TGGGCTGCGTCCTGGATGGGGTGCGCTACAACAACGGCCAGTCCTTCCAGCCTAACTGCAAGTACA
ACTGCACGTGCATCGACGGCGCGGTGGGCTGCACACCACTGTGCCTCCGAGTGCGCCCCCCGCGTCT
CTGGTGCCCCCACCCGCGGCGCGTGAGCATACCTGGCCACTGCTGTGAGCAGTGGGTATGTGAGGA
CGACGCCAAGAGGCCACGCAAGACCGCACCCCGTGACACAGGAGCCTTCGATGCTGTGGGTGAGGT
GGAGGCATGGCACAGGAACTGCATAGCCTACACAAGCCCCTGGAGCCCTTGCTCCACCAGCTGCGG
CCTGGGGGTCTCCACTCGGATCTCCAATGTTAACGCCCAGTGCTGGCCTGAGCAAGAGAGCCGCCTC
TGCAACTTGCGGCCATGCGATGTGGACATCCATACACTCATTAAGGCAGGGAAGAAGTGTCTGGCT
GTGTACCAGCCAGAGGCATCCATGAACTTCACACTTGCGGGCTGCATCAGCACACGCTCCTATCAAC
CCAAGTACTGTGGAGTTTGCATGGACAATAGGTGCTGCATCCCCTACAAGTCTAAGACTATCGACGT
GTCCTTCCAGTGTCCTGATGGGCTTGGCTTCTCCCGCCAGGTCCTATGGATTAATGCCTGCTTCTGTA
ACCTGAGCTGTAGGAATCCCAATGACATCTTTGCTGACTTGGAATCCTACCCTGACTTCTCAGAAAT
TGCCAACTAGGCAGGCACAAATCTTGGGTCTTGGGGACTAACCCAATGCCTGTGAAGCAGTCAGCC
CTTATGGCCAATAACTTTTCACCAATGAGCCTTAGTTACCCTGATCTGGACCCTTGGCCTCCATTTCT
GTCTCTAACCATTCAAATGACGCCTGATGGTGCTGCTCAGGCCCATGCTATGAGTTTTCTCCTTGATA
TCATTCAGCATCTACTCTAAAGAAAAATGCCTGTCTCTAGCTGTTCTGGACTACACCCAAGCCTGAT
CCAGCCTTTCCAAGTCACTAGAAGTCCTGCTGGATCTTGCCTAAATCCCAAGAAATGGAATCAGGTA
GACTTTTAATATCACTAATTTCTTCTTTAGATGCCAAACCACAAGACTCTTTGGGTCCATTCAGATGA
ATAGATGGAATTTGGAACAATAGAATAATCTATTATTTGGAGCCTGCCAAGAGGTACTGTAATGGGT
AATTCTGACGTCAGCGCACCAAAACTATCCTGATTCCAAATATGTATGCACCTCAAGGTCATCAAAC
ATTTGCCAAGTGAGTTGAATAGTTGCTTAATTTTGATTTTTAATGGAAAGTTGTATCCATTAACCTGG
GCATTGTTGAGGTTAAGTTTCTCTTCACCCCTACACTGTGAAGGGTACAGATTAGGTTTGTCCCAGTC
AGAAATAAAATTTGATAAACATTCCTGTTGATGGGAAAAGCCCCCAGTTAATACTCCAGAGACAGG
GAAAGGTCAGCCCGTTTCAGAAGGACCAATTGACTCTCACACTGAATCAGCTGCTGACTGGCAGGG
CTTTGGGCAGTTGGCCAGGCTCTTCCTTGAATCTTCTCCCTTGTCCTGCTTGGGGTTCATAGGAATTG
GTAAGGCCTCTGGACTGGCCTGTCTGGCCCCTGAGAGTGGTGCCCTGGAACACTCCTCTACTCTTAC
AGAGCCTTGAGAGACCCAGCTGCAGACCATGCCAGACCCACTGAAATGACCAAGACAGGTTCAGGT
AGGGGTGTGGGTCAAACCAAGAAGTGGGTGCCCTTGGTAGCAGCCTGGGGTGACCTCTAGAGCTGG
AGGCTGTGGGACTCCAGGGGCCCCCGTGTTCAGGACACATCTATTGCAGAGACTCATTTCACAGCCT
TTCGTTCTGCTGACCAAATGGCCAGTTTTCTGGTAGGAAGATGGAGGTTTACCGGTTGTTTAGAAAC
AGAAATAGACTTAATAAAGGTTTAAAGCTGAAGAGGTTGAAGCTAAAAGGAAAAGGTTGTTGTTAA
TGAATATCAGGCTATTATTTATTGTATTAGGAAAATATAATATTTACTGTTAGAATTCTTTTATTTAG
GGCCTTTTCTGTGCCAGACATTGCTCTCAGTGCTTTGCATGTATTAGCTCACTGAATCTTCACGACAA
TGTTGAGAAGTTCCCATTATTATTTCTGTTCTTACAAATGTGAAACGGAAGCTCATAGAGGTGAGAA
AACTCAACCAGAGTCACCCAGTTGGTGACTGGGAAAGTTAGGATTCAGATCGAAATTGGACTGTCTT
TATAACCCATATTTTCCCCCTGTTTTTAGAGCTTCCAAATGTGTCAGAATAGGAAAACATTGCAATA
AATGGCTTGATTTTTTAATGTCATTTTTCCCTCTTATAGTCTTTCTAGCTCCTTTTCAAAAGACGAGAA
TATCTGATTTTCTGATAATTTAGGTGCTTAAGCATCCAAAATACATGGGACACACAAAAATCCAGGA
ATCCCCTGTAGCTTATTCCCTCTTTCCCATCGGAACCAGCTCTCATCACACATTTAAAAGATGATTCT
GTTTACCCAATGCTGCATATTGAATGTTGTGTAGTTATTCACAGGGAATTCTGTGCAGTGTGCAGAG
AGATTCCTAAACGGGAAAAGGACTGGGAATACATCCTCCTTACTGTGACCTCCCCAAAACCTAGTCC
AGTGCAAGGTATACAGTGGTGCTCATTAAATACTTGATGAATACAGGAAGCTGTGCATGTGTTCCTA
CTTTTATTCGAAGCTCTCTTCTTCCAAAGCTACATGAAAATAGAATTTTAACAGTCAAAATTTTATAT
TAAGTGCCTTAGCAAAAGAGACATTTAATATTTCAAAGAAATGCATATGTATGTATACATATATTTG
TGTATGCGTATGCAAGAATTCTTGTATAAAGAGAATTCACTCCATGAATGATCTCTTCTGTAAGTCA
GTGTGAATCATGTTAGATTTTCTGAGAGTGAAAACACCTGCCATCTACAAATTACAAGGCTGGATAA
CAGCTCACTCCATTTGAAATTCAGTGGAAACCCAAGAGCTAGGTTCTTACTGAATTTGCATCTCAAT
TTGGGAAACTGAACTTAGCTTTCAAAGATCATAGGAAGTCTGGTTGGAGAAACTAGGGATTATTCTG
GCAATGGGTGCAGGAAGGTGGTCAGAATAACCCAGTCGCCATTGGTTTTGAGAAACGGAACTATCT
TATGCAGAGCCCGGAGGGCAAGTCTCAGACCCATGGGTTGAAGCCATGGAGAAGGAAATTTGGATC
CAATGTAATGAAGCGCTTTCTAAGTCAGAATTTCCCTGCAATGGTGTGGCCTGATTCAATAAAAATT
AAGAATAATAAATATAATGGAAAAAAATCTCCACTGATTGAGTGTTTACTTGGTGCCAAGCACTATG
CTAAGTTGTTCATTATTTTATTTAATTGTTACAGCAATTTTGAGTATGCATCTTTCACTATTTTATAAG
TGGAAAAGAGAAGTGCCCCCAAAAAGTTAGAGCTCAAACAGCAGCTTATTCTACCAGCCCCTGCTC
TTGCGGAGGCCTCTGGAAAAGACCTGAATGACACCTATTGGAGAATTACATCTACAAGGGGCTTCA
AACAGACCAAATAGATCATCACCTCTGTGGTCCCTTGTTAACTATATGTTCTGAGACAAAGGAAAGC
TACCCTAAGGGTTAGTTAACCTTTGCTGAGGAAATTTACATTCATACTTAGAGTGAATTACTCAGGT
GTGCTTAGGTGTGCAAAAGGGAAGGAGACCTGAATTCACCAAGTTAAATCTTGCTAAACCTTATCAT
AAGCATTTTTTGAGCGCTTAGCATACACCAAGCCTTGTGGAAGGTGCTTTCCTGCCATATCTCATTTA
ATCCTCACAGCAAACCTATAGAATATGGCATTATCATCTGAGTCTCACAGAAGTTTAGTCGTGTACT
CAAGGTCTTACCAGCTAGTGAACAGCAGACCAAGACTGGAAACCCAGGATAGTCTGATACCTGAGC
CATCTCTTCTTGTGCTACGCCTAGTTATTCTGTCCCCCAAATCAAAAGGCATGACCTTTATAAGAGGC
GCTTTACTGACAATAGCTGCAATTTTAACTTTGAAAATGATTCAGAATTATCAAAGATAGTAGATTC
GAATGACATGATTGTCTATAATCTCGCTAGCCTTGTACTGTGTGTGCATAGCAATTACAGGGAAGTA
ATCTAGCTCCTGACTATTATGTTGAACTATGTCGCTGCTTTTTACAAACTTGTCTTGATCCAAAGCAG
TCACAATGATAACCCTGCATATCTGGGAATCATAAGTCAACTATGTATCCCTGTGTGTGTATATATA
TGTATGTATGTATCTATTTTCAAACTGTGATTTAATATTTAAATATTCCTACTGCCATTTTTGTGACTG
AAAAACTACACATGAGGAAACGTCTTAGAATTTTCCAATAGAGGAAAAATAACACTTGGGCAATCT
GTCATGTTTCACAACAGTTCTCATTTTTCTCATGATTTGTGTAGCGTGGAATGTGTTTGCTCAATGTG
AAGGGTTTTCATTGCTCAATTTCTCTGTGTAAGTCTTTTCCTTAAGGTAATAAACCATCAGCAAAGTC
ACATACTGGAGTTGGTGGCTTTTCTTGTACAGGCAGTTGTTATGAGACAATGATGGAGCATTGAGCA
TGTTCAATAAATGTGCAGATGGTGGAAAAAAA
SEQ ID NO: 6 is an amino acid sequence of mouse WNT1-inducible-
signaling pathway protein 1 precursor [Mus musculus].
MRWLLPWTLAAVAVLRVGNILATALSPTPTTMTFTPAPLEETTTRPEFCKWPCECPQSPPRCPLGVSLIT
DGCECCKICAQQLGDNCTEAAICDPHRGLYCDYSGDRPRYAIGVCAQVVGVGCVLDGVRYTNGESFQP
NCRYNCTCIDGTVGCTPLCLSPRPPRLWCRQPRHVRVPGQCCEQWVCDDDARRPRQTALLDTRAFAAS
GAVE
QRYENCIAYTSPWSPCSTTCGLGISTRISNVNARCWPEQESRLCNLRPCDVDIQLHIKAGKKCLAVYQPE
EATNFTLAGCVSTRTYRPKYCGVCTDNRCCIPYKSKTISVDFQCPEGPGFSRQVLWINACFCNLSCRNPN
DIFADLESYPDFEEIAN
SEQ ID NO: 7 is a nucleotide sequence of mouse WNT1 inducible
signaling pathway protein 1 (Wisp1), transcript variant 1, mRNA
[Mus musculus].
AGAAAAAGTTTTTTTAGAGGAAAATGCAGGGCTAGTCTGTTGGCCTGACGTCAGATGTCGCTTTGAC
AAACGCCCCCGGGGGCTGAGGAAGGCTCTCCGCTGCTCTGATGGGCCAGCCCAGTCCTGGCCCAGC
TCCCTGGAGAGGCATCCGCATCCTCTGGGCTGAGCCGTAGCTCCTGTGACGCTGACTTCCAGGCATG
AGGTGGCTCCTGCCCTGGACGCTGGCAGCCGTGGCAGTCCTGAGGGTGGGCAACATCCTGGCCACG
GCCCTCTCTCCAACCCCCACAACAATGACCTTCACCCCAGCACCACTAGAGGAAACGACTACACGCC
CCGAATTCTGCAAGTGGCCATGTGAGTGCCCACAATCCCCACCTCGCTGCCCACTGGGCGTCAGCCT
AATCACAGATGGCTGTGAATGCTGTAAGATATGTGCCCAGCAGCTTGGGGACAACTGCACAGAGGC
TGCCATCTGTGACCCACACCGGGGCCTCTACTGCGATTACAGTGGGGATCGCCCGAGGTACGCAATA
GGAGTGTGTGCACAGGTGGTCGGTGTGGGCTGTGTCCTGGATGGCGTACGCTACACCAATGGCGAG
TCCTTCCAACCCAACTGCAGGTACAACTGTACCTGCATTGATGGCACGGTGGGCTGCACACCGCTGT
GCCTAAGCCCCAGGCCCCCACGCCTCTGGTGCCGCCAGCCCCGGCACGTGAGAGTCCCTGGCCAGT
GCTGTGAGCAGTGGGTGTGTGATGATGACGCAAGGAGACCACGCCAGACTGCACTGTTGGACACCA
GAGCCTTTGCAGCGTCAGGCGCCGTGGAGCAACGGTATGAGAACTGCATAGCCTACACTAGTCCCT
GGAGCCCCTGCTCTACCACCTGTGGCCTAGGTATCTCCACTCGGATCTCTAACGTCAATGCCCGGTG
CTGGCCAGAGCAGGAAAGTCGCCTCTGCAACCTGCGGCCATGTGATGTGGACATCCAACTACACAT
CAAGGCAGGGAAGAAATGCCTGGCTGTGTACCAGCCAGAGGAGGCCACGAACTTCACTCTCGCAGG
CTGTGTCAGCACACGCACCTACCGACCCAAGTACTGCGGAGTCTGTACTGACAATAGGTGTTGCATC
CCCTACAAGTCCAAGACCATCAGTGTGGATTTCCAGTGTCCAGAGGGGCCAGGTTTCTCCCGGCAGG
TCCTATGGATTAATGCTTGCTTCTGCAACCTGAGCTGCAGGAATCCTAACGATATCTTTGCTGACTTG
GAATCTTACCCTGACTTCGAAGAGATTGCCAATTAGGTGGGTGTGTGGCTCAGGGTAAAGTTCCATG
CTGCAAAGCAGCCAGCCCTTTGTGGTCCAGGACTTCACAATTGAGCCTTATTTCATCTACTTCCTACT
CGATTCTGAATTCCCAGTTTCTGTTCCTGTTTTGACAATCGTAATGGCCCAGGAGAGTGCTGCTCAGG
CTCAGACAATGGGTTCCTCCTTGGGGACATTCTACATCATTCCAAGGAAAACACATCTCTGACTGTT
CACAATGGAAGCAAAGCCTGGCCCAGCTAGTCTGGCTCCAGCCTGGGCAAGTTGTCAGAAGTTGTG
ATGGGATTGTCCAAGGAAAAGCATCAGCTGAAGAACCAGTATCATGAAGTCCTTCCTCAGATGCCA
AGCCTAGGGATGCTGGGATCCTTTCAGACAGATGGATGGGATTGGGGACACAGGAATAAGCTATTA
TTTTACCCTTGCCAAATGATACTATCCTGGGTATTTCTGCCTAAAACATACCAAAAGTGTTCTTGTTC
CACTGATCTGTATATCACAAGTCACCAAACATTTTCCAGGTGAGGACCATAGTTGTGTCATTCTGTTT
TGCTATTGAAAATCATTTTAAAAAGAAGGAAAAAAAAAAAAGAAAAGAAAAGAAAGTCATTTCATT
AACTTGGGCACTTTCTCCTCTCACCCCATATTCTATAAAGGGCTAAACTTGGGTTCTTGTTGATCAGG
AATGTAATTTGAGAAGTCTTACTTTTGCAGGGAGATGGTAGCCCTCAATTCATCCCGTAGGGATGGG
ACAAGGCCAGCCAATCTTTCAAGCCATAGCTGGGCAGGTCACTGAATCTGCTGCTGGCCAAGTTCTT
AGGACAATTAGCCAAAATCTGGGCCTCTCTCTCCCTAGGGTTCATGGGAGTTGGTAGGGACCGTAGA
GTGACTTGTCTGTTGTCTCAAAAAGTAAGATGGAAAGATGTTCTCATGGCCCTTAGAAGACTCTTTT
GAAGTCTACGCCAGACCTAACAGAATATGTGCATCAAACAAACAAGTGGATCACCCTCCCATGGCC
TGGGTGACCTCTAGCAGTCACCCAGTGACTGTGGTAAGGCCACAGTAGTCCCTGGACCCAGGACAA
ATCTTTTTTGTTTCAGTGACCTACTTTACAGCCTCAGTGTCTATGAAGAAAGTTAACTCAGTTTTCTC
ATCTGAAGACAGAAGTCGACCAGCCATTCAGAAATGGGGATCTATTTAGTAGTGGTAAACGTTAAA
GAGTTTGTAAGCTAAAGAAATATTTTTTTTCCTAAGTGATAATGAGGTTGTTACCTATTTTAGAGGTA
GAATTTTCTGTCATCATCCTTTTATTGTGTTCTACATACTACCCATTGTTCTCAATATTTGCCACACAT
TTACTCATTGAAACTTGCTGATAACACAGAGGACTTCCCTTAATGATTTCTGTTCTTGACACTGTGAA
ATATCAATTCAAAGAGGCTAATAAGTTCAATCAAAGTCACCTATCTGAAGACTCATAGCCAGAGAA
GATTGCCTTCATATTTCCCCTTTGCTTCTGGACCTTGTAAACATGTCAGGGCAGGAAAGCATACAGA
AGTCAGCAGCTTGCCCTCTTAGTATTGGCTCTCTAGCCCCTTTCACGTAAGAGGAGAGTGTGTCTAG
GTACTAAGGATCCAACCTAAACTGGACAGAAAAGGGCATGTGCATTATCCCCTGTGGCTCCTCCTGC
CCTGGAATCAGAACTAGCTTACAGCATGCTTTTATATGATGATTCAGTTTGCCCCACACCATACATA
TGGGCATATGGGAGAACTATACACACACTCATAAATGGATTTGTAAGTAGGAAAGGGGACTTGAGA
TTTCCCCTCCTTCCTGCAACTTCCCAAAGGCCTGGCCTTATGCAAGGCAAACAGTGGTGCTTAAGAT
GTATTTTGGGAAGAAAATACTATATATATATATTCCTATTTTTCCTTAGAAGCCTTTTCTTCCAGGAT
CTCATCTTAACAGTAAGAAATCCTCTCTTAAGAGAGGCCTAGTATTTTGAGTAAACATATGTTCATA
TGTGTACAAGTACTTGTATGTAAATATAAGTTACCACTGTGGGTTATTAATTAGGTAAATTTTATGTG
GTATCATATTTGATTTTCTTCTATTGGAAAAATCTGCCTTAACAGCTAACTCTGTAAGAACTTCCATA
GGAGCCATAAGCTAGGGTCTTCCCAGGTATCCATCCTCTTTTGGGAAACTGAGCTGAGCATCTTCAA
CCAAAGGAGTTAGGGTGATCATTGGGAATAGGAGAAAGGGATGGCCAGGGTAGCTCCATCGTTATT
TAGAAACAGACCTGGCATACAGAACGACCAGAGGAACCAACCTTCTTTGAACCAAGGGAAAAAGA
CTTGGATGTAATATATAGAAGCTTTTTCTAATAGTCAGAAACAGACTTTAATTGTATGGCCTGGTTC
AAGGAAAGTTAAGAATGTCCATTATCGTTAAAAACAAAAGTCCATTATTGATAGCTTATTTGGTGCT
AAGCCCTACGGTGCATTTTGTCCGGCTATCACTTAGCAGAGCCATGCTCAGTACACAGTCTTTCTCTA
TTCAATAAATAGAAACGCTGAAGCTCAAAGGCACTGAGGAGCTGAGGCTCAGAAGCATGTTTAGTC
CACTCTATCAGGGGGGGAAGAGATCTTGACGGAACCTAAATGACTACTATTGGAAACTCATATTTG
AAAGCTTTCAGAAGTCCCACCCCCACCCCATCCCCAGATGCTTAGAAACTAAAGAAGCAATGAGGA
TGAGCTAGCCTTCAGTGAAGAGGTTCACTACTGCACCAAGAGTGAATGTCTTAGGTGTACTTAGTCA
TTGGACAGGGAGACCTGAGTGAGTTTGTGAACCTGCAGCTTACTAAACCTTACAATGAGCATTTGGA
GAGCCAAGACTGCTTCTCGGCGCTTTACTGACATGGCTTGCTTAATCTTCTCAGTGAGCCCAAGAGT
CAGGGCGTTACCACTGCCCATTTTAGGGCTGAGAAAGCAAAATCTCCAGGAGTTAAGTGATTTGCTC
AAGTTTTTAACCAACCGAGGCACTGCAGATAAACTCCGAAGCCCAGTGTCATGTAACATGCCCATGC
CATCTCTCCGGACACGCAGCCCATTTTCCTGTTCCTAAACCAAAGGCTCAGAGTCACCAGAACCAAC
TCACAGGACAGTGCAGAAATTCTAATGTCGAGGGTGATTAGAGACTGATCAAAGAAAGTAATTTCA
AATGATATGATTGTTTGTAAGCACCCTAGTTAATTCTGGACTACATATGCATAGAGATTGTGAAGAA
CATTACAGCCTGTGACTATAACGTTGACTTCTGTCATTTCTTTTTAAAGACTTGTTTTTTTTTTTTTAC
TCAAAGGACCCACAGTGACAGCCCTGAATGGTTGAGAAGCATTGATTAGCTGTGAGTCCTGCATAT
GTATGTATGTGTGTGTGTGTGTGTGTATTTGTATGTACTTATCTATTTTCAAACTGTGATTGTGTATTT
AAATATTCCTCCTGCCATTTTGTAAGTGATTACGCATAAAGAAACACCTTTGAATGTCCTAATAAAG
GAGAGCTAGCCCTTGGGCGGCCTGTCACATTTTGCCACTTTCCTCATTTTTCTCATGATCTGTGTAGC
AGGGAATGTGTTTGTTCAACCATGATGAGTTTTCATTGTTCAAATTCTTTGTTTACAGCTTTTCTCCTT
AAAGCAATAAATCATCAGCAACAGTAA
SEQ ID NO: 8 is a nucleotide sequence of human MiR 15a.
(SEQ ID NO: 8)
CCUUGGAGUAAAGUAGCAGCACAUAAUGGUUUGUGGAUUUUGAAAAGGUGCAGGCCAUAUUGUGCUGCCU
CAAAAAUACAAGG
SEQ ID NO: 9 is a nucleotide sequence of human Mir-412.
(SEQ ID NO: 9)
CUGGGGUACGGGGAUGGAUGGUCGACCAGUUGGAAAGUAAUUGUUUCUAAUGUACUUCACCUGGUCCACU
AGCCGUCCGUAUCCGCUGCAG
SEQ ID NO: 10 is a nucleotide sequence of mouse MiR 15a.
(SEQ ID NO: 10)
CCCUUGGAGUAAAGUAGCAGCACAUAAUGGUUUGUGGAUGUUGAAAAGGUGCAGGCCAUACUGUGCUGCC
UCAAAAUACAAGGA
SEQ ID NO: 11 is a nucleotide sequence of mouse Mir-412.
(SEQ ID NO: 11)
GGGUAUGGGACGGAUGGUCGACCAGCUGGAAAGUAAUUGUUUCUAAUGUACUUCACCUGGUCCACUAGCC
GUCGGUGCCC

SEQ ID NOs: 12-31 are Rattus heavy chain complementarity determining regions for IgG.

| SEQ ID NO: | Accession | Name | Organism name | Length |
|---|---|---|---|---|
| 12 | 1bfo_B | CAMPATH-1G IGG2B RAT | Rattus rattus | 216 |
| | | MONOCLONAL FAB | | |
| 13 | 1bfo_D | CAMPATH-1G IGG2B RAT | Rattus rattus | 216 |
| | | MONOCLONAL FAB | | |
| 14 | 1bfo_F | CAMPATH-1G IGG2B RAT | Rattus rattus | 216 |
| | | MONOCLONAL FAB | | |
| 15 | 1bfo_H | CAMPATH-1G IGG2B RAT | Rattus rattus | 216 |
| | | MONOCLONAL FAB | | |
| 16 | 1c5d_B | THE CRYSTAL STRUCTURE OF THE | Rattus norvegicus | 215 |
| | | FAB FRAGMENT OF A RAT | | |
| | | MONOCLONAL ANTIBODY AGAINST | | |
| | | THE MAIN IMMUNOGENIC REGION | | |
| | | OF THE HUMAN MUSCLE | | |
| | | ACETYLCHOLINE RECEPTOR | | |
| 17 | 1c5d_H | THE CRYSTAL STRUCTURE OF THE | Rattus norvegicus | 215 |
| | | FAB FRAGMENT OF A RAT | | |
| | | MONOCLONAL ANTIBODY AGAINST | | |
| | | THE MAIN IMMUNOGENIC REGION | | |
| | | OF THE HUMAN MUSCLE | | |
| | | ACETYLCHOLINE RECEPTOR | | |
| 18 | 1f3r_B_i | COMPLEX BETWEEN FV ANTIBODY | Rattus norvegicus | 138 |
| | | FRAGMENT AND AN ANALOGUE OF | | |
| | | THE MAIN IMMUNOGENIC REGION | | |
| | | OF THE ACETYLCHOLINE RECEPTOR | | |
| 19 | 1fn4_B | CRYSTAL STRUCTURE OF FAB198, AN | Rattus norvegicus | 218 |
| | | EFFICIENT PROTECTOR OF | | |
| | | ACETYLCHOLINE RECEPTOR | | |
| | | AGAINST MYASTHENOGENIC | | |
| | | ANTIBODIES | | |
| 20 | 1fn4_D | CRYSTAL STRUCTURE OF FAB198, AN | Rattus norvegicus | 218 |
| | | EFFICIENT PROTECTOR OF | | |
| | | ACETYLCHOLINE RECEPTOR | | |
| | | AGAINST MYASTHENOGENIC | | |
| | | ANTIBODIES | | |
| 21 | 2arj_B | CD8ALPHA-ALPHA IN COMPLEX | Rattus norvegicus | 215 |
| | | WITH YTS 105.18 FAB | | |
| 22 | 2arj_H | CD8ALPHA-ALPHA IN COMPLEX | Rattus norvegicus | 215 |
| | | WITH YTS 105.18 FAB | | |
| 23 | 3iy7_B | VARIABLE DOMAINS OF THE | Rattus norvegicus | 115 |
| | | COMPUTER GENERATED MODEL | | |
| | | (WAM) OF FAB F FITTED INTO THE | | |
| | | CRYOEM RECONSTRUCTION OF THE | | |
| | | VIRUS-FAB F COMPLEX | | |
| 24 | 4why_G | STRUCTURE OF THE HEPATITIS C | Rattus norvegicus | 252 |
| | | VIRUS ENVELOPE GLYCOPROTEIN E2 | | |
| | | ANTIGENIC REGION 412-423 BOUND | | |
| | | TO THE BROADLY NEUTRALIZING | | |
| | | ANTIBODY 3/11, P21 CRYSTAL FORM | | |
| 25 | 4why_I | STRUCTURE OF THE HEPATITIS C | Rattus norvegicus | 252 |
| | | VIRUS ENVELOPE GLYCOPROTEIN E2 | | |
| | | ANTIGENIC REGION 412-423 BOUND | | |
| | | TO THE BROADLY NEUTRALIZING | | |
| | | ANTIBODY 3/11, P21 CRYSTAL FORM | | |
| 26 | 4why_K | STRUCTURE OF THE HEPATITIS C | Rattus norvegicus | 252 |
| | | VIRUS ENVELOPE GLYCOPROTEIN E2 | | |
| | | ANTIGENIC REGION 412-423 BOUND | | |
| | | TO THE BROADLY NEUTRALIZING | | |
| | | ANTIBODY 3/11, P21 CRYSTAL FORM | | |
| 27 | 4yny_A | CRYSTAL STRUCTURE OF | Rattus norvegicus | 238 |
| | | MONOCLONAL ANTI-HUMAN | | |
| | | PODOPLANIN ANTIBODY NZ-1 | | |
| 28 | 4yny_C | CRYSTAL STRUCTURE OF | Rattus norvegicus | 238 |
| | | MONOCLONAL ANTI-HUMAN | | |
| | | PODOPLANIN ANTIBODY NZ-1 | | |
| 29 | 5aum_A | CRYSTAL STRUCTURE OF A FAB | Rattus | 242 |
| | | FRAGMENT WITH THE LIGAND | | |
| | | PEPTIDE | | |
| 30 | 5aum_H | CRYSTAL STRUCTURE OF A FAB | Rattus | 242 |
| | | FRAGMENT WITH THE LIGAND | | |
| | | PEPTIDE | | |
| 31 | 5hbv_D | COMPLEX STRUCTURE OF FAB35 | Rattus norvegicus | 219 |
| | | AND MOUSE NACHR ALPHA1 | | |

SEQ ID NOs: 32-51 are Rattus light chain complementarity determining regions for IgG.

| SEQ ID NO: | Accession | Name | Organism name | Length |
|---|---|---|---|---|
| 32 | 1bfo_A | CAMPATH-1G IGG2B RAT MONOCLONAL | Rattus rattus | 214 |
| | | FAB | | |
| 33 | 1bfo_C | CAMPATH-1G IGG2B RAT MONOCLONAL | Rattus rattus | 214 |
| | | FAB | | |
| 34 | 1bfo_E | CAMPATH-1G IGG2B RAT MONOCLONAL | Rattus rattus | 214 |
| | | FAB | | |
| 35 | 1bfo_G | CAMPATH-1G IGG2B RAT MONOCLONAL | Rattus rattus | 214 |
| | | FAB | | |
| 36 | 1c5d_A | THE CRYSTAL STRUCTURE OF THE FAB | Rattus norvegicus | 213 |
| | | FRAGMENT OF A RAT MONOCLONAL | | |
| | | ANTIBODY AGAINST THE MAIN | | |
| | | IMMUNOGENIC REGION OF THE HUMAN | | |
| | | MUSCLE ACETYLCHOLINE RECEPTOR | | |
| 37 | 1c5d_L | THE CRYSTAL STRUCTURE OF THE FAB | Rattus norvegicus | 213 |
| | | FRAGMENT OF A RAT MONOCLONAL | | |
| | | ANTIBODY AGAINST THE MAIN | | |
| | | IMMUNOGENIC REGION OF THE HUMAN | | |
| | | MUSCLE ACETYLCHOLINE RECEPTOR | | |
| 38 | 1f3r_B_ii | COMPLEX BETWEEN FV ANTIBODY | Rattus norvegicus | 119 |
| | | FRAGMENT AND AN ANALOGUE OF THE | | |
| | | MAIN IMMUNOGENIC REGION OF THE | | |
| | | ACETYLCHOLINE RECEPTOR | | |
| 39 | 1fn4_A | CRYSTAL STRUCTURE OF FAB198, AN | Rattus norvegicus | 211 |
| | | EFFICIENT PROTECTOR OF | | |
| | | ACETYLCHOLINE RECEPTOR AGAINST | | |
| | | MYASTHENOGENIC ANTIBODIES | | |
| 40 | 1fn4_C | CRYSTAL STRUCTURE OF FAB198, AN | Rattus norvegicus | 211 |
| | | EFFICIENT PROTECTOR OF | | |
| | | ACETYLCHOLINE RECEPTOR AGAINST | | |
| | | MYASTHENOGENIC ANTIBODIES | | |
| 41 | 2arj_A | CD8ALPHA-ALPHA IN COMPLEX WITH | Rattus norvegicus | 211 |
| | | YTS 105.18 FAB | | |
| 42 | 2arj_L | CD8ALPHA-ALPHA IN COMPLEX WITH | Rattus norvegicus | 211 |
| | | YTS 105.18 FAB | | |
| 43 | 3iy7_A | VARIABLE DOMAINS OF THE COMPUTER | Rattus norvegicus | 106 |
| | | GENERATED MODEL (WAM) OF FAB F | | |
| | | FITTED INTO THE CRYOEM | | |
| | | RECONSTRUCTION OF THE VIRUS-FAB F | | |
| | | COMPLEX | | |
| 44 | 4why_H | STRUCTURE OF THE HEPATITIS C VIRUS | Rattus norvegicus | 220 |
| | | ENVELOPE GLYCOPROTEIN E2 | | |
| | | ANTIGENIC REGION 412-423 BOUND TO | | |
| | | THE BROADLY NEUTRALIZING | | |
| | | ANTIBODY 3/11, P21 CRYSTAL FORM | | |
| 45 | 4why_J | STRUCTURE OF THE HEPATITIS C VIRUS | Rattus norvegicus | 220 |
| | | ENVELOPE GLYCOPROTEIN E2 | | |
| | | ANTIGENIC REGION 412-423 BOUND TO | | |
| | | THE BROADLY NEUTRALIZING | | |
| | | ANTIBODY 3/11, P21 CRYSTAL FORM | | |
| 46 | 4why_L | STRUCTURE OF THE HEPATITIS C VIRUS | Rattus norvegicus | 220 |
| | | ENVELOPE GLYCOPROTEIN E2 | | |
| | | ANTIGENIC REGION 412-423 BOUND TO | | |
| | | THE BROADLY NEUTRALIZING | | |
| | | ANTIBODY 3/11, P21 CRYSTAL FORM | | |
| 47 | 4yny_B | CRYSTAL STRUCTURE OF MONOCLONAL | Rattus norvegicus | 233 |
| | | ANTI-HUMAN PODOPLANIN ANTIBODY | | |
| | | NZ-1 | | |
| 48 | 4yny_D | CRYSTAL STRUCTURE OF MONOCLONAL | Rattus norvegicus | 233 |
| | | ANTI-HUMAN PODOPLANIN ANTIBODY | | |
| | | NZ-1 | | |
| 49 | 5aum_B | CRYSTAL STRUCTURE OF A FAB | Rattus | 239 |
| | | FRAGMENT WITH THE LIGAND PEPTIDE | | |
| 50 | 5aum_L | CRYSTAL STRUCTURE OF A FAB | Rattus | 239 |
| | | FRAGMENT WITH THE LIGAND PEPTIDE | | |
| 51 | 5hbv_C | COMPLEX STRUCTURE OF FAB35 AND | Rattus norvegicus | 213 |
| | | MOUSE NACHR ALPHA1 | | |

SEQ ID NOs: 52-71 are Ovis aries (sheep) heavy chain complementarity determining regions for IgG.

HEAVY

| SEQ ID NO: | Accession | Name | Clone | Organism name | Length |
|---|---|---|---|---|---|
| 52 | AAD52588.1 | immunoglobulin heavy chain precursor | 14 | Ovis aries | 124 |
| 53 | AAD52589.1 | immunoglobulin heavy chain precursor | 17 | Ovis aries | 115 |
| 54 | AAD52590.1 | immunoglobulin heavy chain precursor | 22 | Ovis aries | 114 |
| 55 | AAD52591.1 | immunoglobulin heavy chain precursor | 23 | Ovis aries | 120 |
| 56 | AAD52592.1 | immunoglobulin heavy chain precursor | 34 | Ovis aries | 120 |
| 57 | AAD52593.1 | immunoglobulin heavy chain precursor | 35 | Ovis aries | 119 |
| 58 | AAD52594.1 | immunoglobulin heavy chain precursor | 47 | Ovis aries | 122 |
| 59 | AAD52595.1 | immunoglobulin heavy chain precursor | 52 | Ovis aries | 121 |
| 60 | AAD52596.1 | immunoglobulin heavy chain precursor | 53 | Ovis aries | 114 |
| 61 | AAD52597.1 | immunoglobulin heavy chain precursor | 57 | Ovis aries | 122 |
| 62 | AAD52598.1 | immunoglobulin heavy chain precursor | 58 | Ovis aries | 120 |
| 63 | AAD52599.1 | immunoglobulin heavy chain precursor | 70 | Ovis aries | 114 |
| 64 | AAD52600.1 | immunoglobulin heavy chain precursor | 81 | Ovis aries | 116 |
| 65 | AAD52601.1 | immunoglobulin heavy chain precursor | 92 | Ovis aries | 122 |
| 66 | AAD52602.1 | immunoglobulin heavy chain precursor | 96 | Ovis aries | 118 |
| 67 | AAD52603.1 | immunoglobulin heavy chain precursor | 100 | Ovis aries | 117 |
| 68 | AAD52604.1 | immunoglobulin heavy chain precursor | 138 | Ovis aries | 124 |
| 69 | AAD52605.1 | immunoglobulin heavy chain precursor | 139 | Ovis aries | 114 |
| 70 | AAD52606.1 | immunoglobulin heavy chain precursor | 146 | Ovis aries | 123 |
| 71 | CAA89052.1 | VH region precursor | VRB7 | Ovis aries | 143 |

SEQ ID NOs: 72-90 are Ovis aries (sheep) light chain complementarity determining regions for IgG.

LIGHT

| SEQ ID NO: | Accession | Name | Clone | Organism name | Length |
|---|---|---|---|---|---|
| 72 | AAD51674.1 | immunoglobulin light chain variable region | 17 | Ovis aries | 111 |
| 73 | AAD51675.1 | immunoglobulin light chain variable region | 35 | Ovis aries | 113 |
| 74 | AAD51676.1 | immunoglobulin light chain variable region | 14 | Ovis aries | 110 |
| 75 | AAD51677.1 | immunoglobulin light chain variable region | 146 | Ovis aries | 110 |
| 76 | AAD51678.1 | immunoglobulin light chain variable region | 23 | Ovis aries | 112 |
| 77 | AAD51679.1 | immunoglobulin light chain variable region | 34 | Ovis aries | 111 |
| 78 | AAD51680.1 | immunoglobulin light chain variable region | 100 | Ovis aries | 111 |
| 79 | AAD51681.1 | immunoglobulin light chain variable region | 81 | Ovis aries | 111 |
| 80 | AAD51682.1 | immunoglobulin light chain variable region | 22 | Ovis aries | 110 |
| 81 | AAD51684.1 | immunoglobulin light chain variable region | 47 | Ovis aries | 113 |
| 82 | AAD51685.1 | immunoglobulin light chain variable region | 96 | Ovis aries | 111 |
| 83 | AAD51686.1 | immunoglobulin light chain variable region | 53 | Ovis aries | 110 |
| 84 | AAD51687.1 | immunoglobulin light chain variable region | 58 | Ovis aries | 111 |
| 85 | AAD51688.1 | immunoglobulin light chain variable region | 138 | Ovis aries | 112 |
| 86 | AAD51689.1 | immunoglobulin light chain variable region | 92 | Ovis aries | 112 |
| 87 | AAD51690.1 | immunoglobulin light chain variable region | 70 | Ovis aries | 108 |
| 88 | AAD51691.1 | immunoglobulin light chain variable region | 57 | Ovis aries | 111 |
| 89 | AAD51692.1 | immunoglobulin light chain variable region | 139 | Ovis aries | 108 |
| 90 | AAD51693.1 | immunoglobulin light chain variable region | 52 | Ovis aries | 110 |

SEQ ID NOs: 91-110 are Capra hircus (goat) heavy chain complementarity determining regions for IgG.

HEAVY

| | | | | Organism | |
|---|---|---|---|---|---|
| SEQ ID NO: | Accession | Name | Clone | name | Length |
| 91 | ABX89999.1 | immunoglobulin mu heavy chain variable region | 2M2 | Capra hircus | 137 |
| 92 | ABX90000.1 | immunoglobulin mu heavy chain variable region | 2M3 | Capra hircus | 146 |
| 93 | ABX90001.1 | immunoglobulin mu heavy chain variable region | 2M4 | Capra hircus | 149 |
| 94 | ABX90002.1 | immunoglobulin mu heavy chain variable region | 2M5 | Capra hircus | 130 |
| 95 | ABX90003.1 | immunoglobulin mu heavy chain variable region | 2M6 | Capra hircus | 141 |
| 96 | ABX90004.1 | immunoglobulin mu heavy chain variable region | 3Y1 | Capra hircus | 137 |
| 97 | ABX90005.1 | immunoglobulin mu heavy chain variable region | 3Y2 | Capra hircus | 137 |
| 98 | ABX90006.1 | immunoglobulin mu heavy chain variable region | 3Y3 | Capra hircus | 137 |
| 99 | ABX90007.1 | immunoglobulin mu heavy chain variable region | 3Y4 | Capra hircus | 143 |
| 100 | ABX90008.1 | immunoglobulin mu heavy chain variable region | 3Y5 | Capra hircus | 140 |
| 101 | ABX90009.1 | immunoglobulin mu heavy chain variable region | 3Y8 | Capra hircus | 136 |
| 102 | ABX90010.1 | immunoglobulin mu heavy chain variable region | 3Y9 | Capra hircus | 130 |
| 103 | ABX90011.1 | immunoglobulin mu heavy chain variable region | 3Y10 | Capra hircus | 140 |
| 104 | ABX90012.1 | immunoglobulin mu heavy chain variable region | 3Y11 | Capra hircus | 142 |
| 105 | ABX90013.1 | immunoglobulin mu heavy chain variable region | 3Y12 | Capra hircus | 136 |
| 106 | ABY65924.1 | immunoglobulin heavy chain variable region precursor | 1M1 | Capra hircus | 145 |
| 107 | ABY65925.1 | immunoglobulin heavy chain variable region precursor | 1M2 | Capra hircus | 131 |
| 108 | ABY65926.1 | immunoglobulin heavy chain variable region precursor | 1M3 | Capra hircus | 145 |
| 109 | ABY65927.1 | immunoglobulin heavy chain variable region precursor | 1M4 | Capra hircus | 143 |
| 110 | ABY65928.1 | immunoglobulin heavy chain variable region precursor | 1M5 | Capra hircus | 132 |

SEQ ID NOs: 111-121 are Leporidae (rabbit) heavy chain complementarity determining regions for IgG.

HEAVY

| SEQ ID NO: | Accession | Name | Organism name | Length |
|---|---|---|---|---|
| 111 | 004492 | BS-1 | Leporidae | 107 |
| 112 | 004493 | K-25 | Leporidae | 117 |
| 113 | 004515 | BS-5 | Leporidae | 114 |
| 114 | 004520 | 3374 | Leporidae | 104 |
| 115 | 004604 | 3547 | Leporidae | 101 |
| 116 | 004608 | 120 | Leporidae | 49 |
| 117 | 004614 | K4820 | Leporidae | 29 |
| 118 | 004619 | K29-213 | Leporidae | 25 |
| 119 | 004623 | 2717 | Leporidae | 14 |
| 120 | 004624 | XP-1 | Leporidae | 14 |
| 121 | 004628 | AH80-5 | Leporidae | 11 |

SEQ ID NOs: 122-141 are Leporidae (rabbit) light chain complementarity determining regions for IgG.

LIGHT

| SEQ ID NO: | Accession | Name | Organism name | Length |
|---|---|---|---|---|
| 122 | 7173 | K29-213 | Leporidae | 110 |
| 123 | 7174 | AH80-5 | Leporidae | 115 |
| 124 | 7185 | K4820 | Leporidae | 110 |
| 125 | 7187 | BS-1 | Leporidae | 109 |
| 126 | 7188 | BS-5 | Leporidae | 109 |
| 127 | 7189 | 3547 | Leporidae | 109 |
| 128 | 7193 | 3374 | Leporidae | 110 |
| 129 | 7202 | K-25 | Leporidae | 109 |
| 130 | 7204 | 2717 | Leporidae | 111 |
| 131 | 7205 | 120 | Leporidae | 109 |
| 132 | 7211 | XP-1 | Leporidae | 98 |
| 133 | 48258 | k-176′CL | Leporidae | 111 |
| 134 | 48259 | k-188′CL | Leporidae | 112 |
| 135 | 48260 | k-217′CL | Leporidae | 110 |
| 136 | 48261 | k-221′CL | Leporidae | 110 |
| 137 | 48262 | k-227′CL | Leporidae | 112 |
| 138 | 48263 | k-235′CL | Leporidae | 113 |
| 139 | 48264 | k-237′CL | Leporidae | 109 |
| 140 | 48265 | k-246′CL | Leporidae | 111 |
| 141 | 48266 | k-248′CL | Leporidae | 111 |

1-60. (canceled)

61. A method of treating a liver disease, comprising:
administering an antibody or antibody fragment to a subject in need thereof, wherein the antibody or antibody fragment binds and inhibits WNT1-inducible-signaling pathway protein 1 (WISP1), and wherein the liver disease comprises primary biliary cholangitis (PBC), non-alcoholic steatohepatitis (NASH), non-alcoholic fatty liver disease (NAFLD), or primary sclerosing cholangitis (PSC).

62. The method of claim 61, wherein the WISP1 comprises a splice variant selected from the group consisting of: WISP1v, WISP1vx, and WISP1delta exon 3-4.

63. The method of claim 61, wherein the antibody or antibody fragment comprises at least 95% homology to any of SEQ ID NOs: 1-4, 6 or 12-120.

64. The method of claim 61, wherein the antibody or antibody fragment is selected from the group consisting of: mab1680, AF1680, SAB2501114, ab60114, and ab65943.

65. The method of claim 61, wherein the antibody or antibody fragment is formulated for administration by direct injection, subcutaneous injection, muscular injection, or nasal administration.

66. The method of claim 61, wherein WISP1 is inhibited in a target cell of the subject.

67. The method of claim 66, wherein the target cell is a hepatic stellate cell, a fibroblast, or a myofibroblast.

68. The method of claim 61, wherein the antibody or antibody fragment that binds and inhibits WISP1 inhibits WISP1 activity and/or reduces WISP1 protein levels.

69. The method of claim 68, wherein the WISP1 activity is inhibited by at least 50%, relative to an appropriate control.

70. The method of claim 68, wherein the WISP1 protein level is reduced by at least 50%, relative to an appropriate control.

71. The method of claim 61, further comprising: detecting a level of WISP1, Yap, Colla1, and/or Acta2 in a biological sample of the subject prior to the administration.

72. The method of claim 71, further comprising: comparing the level of WISP1, Yap, Colla1, and/or Acta2 in the biological sample of the subject to a reference level, and identifying the subject as having increased WISP1, Yap, Colla1, and/or Acta2 relative to the reference level.

73. The method of claim 72, further comprising: identifying the subject as having the liver disease based on the increased WISP1, Yap, Colla1, and/or Acta2.

74. The method of claim 71, wherein the biological sample comprises a blood sample, tissue sample, buffy coat sample, or serum sample.

75. The method of claim 61, wherein the antibody or antibody fragment is an antibody fragment.

76. A method of treating a liver disease, comprising:
administering an agent to a subject in need thereof, wherein the agent inhibits WNT1-inducible-signaling pathway protein 1 (WISP1) expression and comprises a genome editing system or inhibitory RNA, and wherein the liver disease comprises primary biliary cholangitis (PBC), non-alcoholic steatohepatitis (NASH), non-alcoholic fatty liver disease (NAFLD), or primary sclerosing cholangitis (PSC).

77. The method of claim 76, wherein the inhibitory RNA comprises an siRNA or miRNA.

78. The method of claim 77, wherein the inhibitory RNA comprises the miRNA, and wherein the miRNA comprises miR15a or miR412.

79. A method of generating an engineered hepatic stellate cell that expresses an agent that inhibits WNT1-inducible-signaling pathway protein 1 (WISP1), the method comprising:
contacting the cell with a nucleic acid that encodes the agent; and
culturing the cell for a sufficient time to allow for expression of the agent.

80. The method of claim 79, wherein the agent comprises a genome editing system or inhibitory RNA.


SEQUENCE LISTING (148 sequences)

SEQ ID NO: 1 - protein, 367 aa, Homo sapiens
MRWFLPWTLAAVTAAAASTVLATALSPAPTTMDFTPAPLEDTSSRPQFCKWPCECPPSPPRCPLGVSLITDGCECCKMCAQQLGDNCTEAAICDPHRGLYCDYSGDRPRYAIGVCAQVVGVGCVLDGVRYNNGQSFQPNCKYNCTCIDGAVGCTPLCLRVRPPRLWCPHPRRVSIPGHCCEQWVCEDDAKRPRKTAPRDTGAFDAVGEVEAWHRNCIAYTSPWSPCSTSCGLGVSTRISNVNAQCWPEQESRLCNLRPCDVDIHTLIKAGKKCLAVYQPEASMNFTLAGCISTRSYQPKYCGVCMDNRCCIPYKSKTIDVSFQCPDGLGFSRQVLWINACFCNLSCRNPNDIFADLESYPDFSEIAN

SEQ ID NO: 2 - protein, 280 aa, Homo sapiens
MRWFLPWTLAAVTAAAASTVLATALSPAPTTMDFTPAPLEDTSSRPQFCKWPCECPPSPPRCPLGVSLITDGCECCKMCAQQLGDNCTEAAICDPHRGLYCDYSGDRPRYAIGVCAHAVGEVEAWHRNCIAYTSPWSPCSTSCGLGVSTRISNVNAQCWPEQESRLCNLRPCDVDIHTLIKAGKKCLAVYQPEASMNFTLAGCISTRSYQPKYCGVCMDNRCCIPYKSKTIDVSFQCPDGLGFSRQVLWINACFCNLSCRNPNDIFADLESYPDFSEIAN

SEQ ID NO: 3 - protein, 155 aa, Homo sapiens
MRWFLPWTLAAVTAAAASTVLATALSPAPTTMDFTPAPLEDTSSRPQFCKWPCECPPSPPRCPLGVSLITDGCECCKMCAQQLGDNCTEAAICDPHRGLYCDYSGDRPRYAIGVCARREEVSGCVPARGIHELHTCGLHQHTLLSTQVLWSLHGQ

SEQ ID NO: 4 - protein, 122 aa, Homo sapiens
MRWFLPWTLAAVTAAAASTVLATAGKKCLAVYQPEASMNFTLAGCISTRSYQPKYCGVCMDNRCCIPYKSKTIDVSFQCPDGLGFSRQVLWINACFCNLSCRNPNDIFADLESYPDFSEIAN

SEQ ID NO: 5 - DNA, 5194 bp, Homo sapiens
  source 1..5194: mol_type = genomic DNA
atatctggtgctcctgatgggccggccagtctgggcccagctcccccgagaggtggtcggatcctctgggctgctcggtcgatgcctgtgccactgacgtccaggcatgaggtggttcctgccctggacgctggcagcagtgacagcagcagccgccagcaccgtcctggccacggccctctctccagcccctacgaccatggactttaccccagctccactggaggacacctcctcacgcccccaattctgcaagtggccatgtgagtgcccgccatccccaccccgctgcccgctgggggtcagcctcatcacagatggctgtgagtgctgtaagatgtgcgctcagcagcttggggacaactgcacggaggctgccatctgtgacccccaccggggcctctactgtgactacagcggggaccgcccgaggtacgcaataggagtgtgtgcacaggtggtcggtgtgggctgcgtcctggatggggtgcgctacaacaacggccagtccttccagcctaactgcaagtacaactgcacgtgcatcgacggcgcggtgggctgcacaccactgtgcctccgagtgcgccccccgcgtctctggtgcccccacccgcggcgcgtgagcatacctggccactgctgtgagcagtgggtatgtgaggacgacgccaagaggccacgcaagaccgcaccccgtgacacaggagccttcgatgctgtgggtgaggtggaggcatggcacaggaactgcatagcctacacaagcccctggagcccttgctccaccagctgcggcctgggggtctccactcggatctccaatgttaacgcccagtgctggcctgagcaagagagccgcctctgcaacttgcggccatgcgatgtggacatccatacactcattaaggcagggaagaagtgtctggctgtgtaccagccagaggcatccatgaacttcacacttgcgggctgcatcagcacacgctcctatcaacccaagtactgtggagtttgcatggacaataggtgctgcatcccctacaagtctaagactatcgacgtgtccttccagtgtcctgatgggcttggcttctcccgccaggtcctatggattaatgcctgcttctgtaacctgagctgtaggaatcccaatgacatctttgctgacttggaatcctaccctgacttctcagaaattgccaactaggcaggcacaaatcttgggtcttggggactaacccaatgcctgtgaagcagtcagcccttatggccaataacttttcaccaatgagccttagttaccctgatctggacccttggcctccatttctgtctctaaccattcaaatgacgcctgatggtgctgctcaggcccatgctatgagttttctccttgatatcattcagcatctactctaaagaaaaatgcctgtctctagctgttctggactacacccaagcctgatccagcctttccaagtcactagaagtcctgctggatcttgcctaaatcccaagaaatggaatcaggtagacttttaatatcactaatttcttctttagatgccaaaccacaagactctttgggtccattcagatgaatagatggaatttggaacaatagaataatctattatttggagcctgccaagaggtactgtaatgggtaattctgacgtcagcgcaccaaaactatcctgattccaaatatgtatgcacctcaaggtcatcaaacatttgccaagtgagttgaatagttgcttaattttgatttttaatggaaagttgtatccattaacctgggcattgttgaggttaagtttctcttcacccctacactgtgaagggtacagattaggtttgtcccagtcagaaataaaatttgataaacattcctgttgatgggaaaagcccccagttaatactccagagacagggaaaggtcagcccgtttcagaaggaccaattgactctcacactgaatcagctgctgactggcagggctttgggcagttggccaggctcttccttgaatcttctcccttgtcctgcttggggttcataggaattggtaaggcctctggactggcctgtctggcccctgagagtggtgccctggaacactcctctactcttacagagccttgagagacccagctgcagaccatgccagacccactgaaatgaccaagacaggttcaggtaggggtgtgggtcaaaccaagaagtgggtgcccttggtagcagcctggggtgacctctagagctggaggctgtgggactccaggggcccccgtgttcaggacacatctattgcagagactcatttcacagcctttcgttctgctgaccaaatggccagttttctggtaggaagatggaggtttaccggttgtttagaaacagaaatagacttaataaaggtttaaagctgaagaggttgaagctaaaaggaaaaggttgttgttaatgaatatcaggctattatttattgtattaggaaaatataatatttactgttagaattcttttatttagggccttttctgtgccagacattgctctcagtgctttgcatgtattagctcactgaatcttcacgacaatgttgagaagttcccattattatttctgttcttacaaatgtgaaacggaagctcatagaggtgagaaaactcaaccagagtcacccagttggtgactgggaaagttaggattcagatcgaaattggactgtctttataacccatattttccccctgtttttagagcttccaaatgtgtcagaataggaaaacattgcaataaatggcttgattttttaatgtcatttttccctcttatagtctttctagctccttttcaaaagacgagaatatctgattttctgataatttaggtgcttaagcatccaaaatacatgggacacacaaaaatccaggaatcccctgtagcttattccctctttcccatcggaaccagctctcatcacacatttaaaagatgattctgtttacccaatgctgcatattgaatgttgtgtagttattcacagggaattctgtgcagtgtgcagagagattcctaaacgggaaaaggactgggaatacatcctccttactgtgacctccccaaaacctagtccagtgcaaggtatacagtggtgctcattaaatacttgatgaatacaggaagctgtgcatgtgttcctacttttattcgaagctctcttcttccaaagctacatgaaaatagaattttaacagtcaaaattttatattaagtgccttagcaaaagagacatttaatatttcaaagaaatgcatatgtatgtatacatatatttgtgtatgcgtatgcaagaattcttgtataaagagaattcactccatgaatgatctcttctgtaagtcagtgtgaatcatgttagattttctgagagtgaaaacacctgccatctacaaattacaaggctggataacagctcactccatttgaaattcagtggaaacccaagagctaggttcttactgaatttgcatctcaatttgggaaactgaacttagctttcaaagatcataggaagtctggttggagaaactagggattattctggcaatgggtgcaggaaggtggtcagaataacccagtcgccattggttttgagaaacggaactatcttatgcagagcccggagggcaagtctcagacccatgggttgaagccatggagaaggaaatttggatccaatgtaatgaagcgctttctaagtcagaatttccctgcaatggtgtggcctgattcaataaaaattaagaataataaatataatggaaaaaaatctccactgattgagtgtttacttggtgccaagcactatgctaagttgttcattattttatttaattgttacagcaattttgagtatgcatctttcactattttataagtggaaaagagaagtgcccccaaaaagttagagctcaaacagcagcttattctaccagcccctgctcttgcggaggcctctggaaaagacctgaatgacacctattggagaattacatctacaaggggcttcaaacagaccaaatagatcatcacctctgtggtcccttgttaactatatgttctgagacaaaggaaagctaccctaagggttagttaacctttgctgaggaaatttacattcatacttagagtgaattactcaggtgtgcttaggtgtgcaaaagggaaggagacctgaattcaccaagttaaatcttgctaaaccttatcataagcattttttgagcgcttagcatacaccaagccttgtggaaggtgctttcctgccatatctcatttaatcctcacagcaaacctatagaatatggcattatcatctgagtctcacagaagtttagtcgtgtactcaaggtcttaccagctagtgaacagcagaccaagactggaaacccaggatagtctgatacctgagccatctcttcttgtgctacgcctagttattctgtcccccaaatcaaaaggcatgacctttataagaggcgctttactgacaatagctgcaattttaactttgaaaatgattcagaattatcaaagatagtagattcgaatgacatgattgtctataatctcgctagccttgtactgtgtgtgcatagcaattacagggaagtaatctagctcctgactattatgttgaactatgtcgctgctttttacaaacttgtcttgatccaaagcagtcacaatgataaccctgcatatctgggaatcataagtcaactatgtatccctgtgtgtgtatatatatgtatgtatgtatctattttcaaactgtgatttaatatttaaatattcctactgccatttttgtgactgaaaaactacacatgaggaaacgtcttagaattttccaatagaggaaaaataacacttgggcaatctgtcatgtttcacaacagttctcatttttctcatgatttgtgtagcgtggaatgtgtttgctcaatgtgaagggttttcattgctcaatttctctgtgtaagtcttttccttaaggtaataaaccatcagcaaagtcacatactggagttggtggcttttcttgtacaggcagttgttatgagacaatgatggagcattgagcatgttcaataaatgtgcagatggtggaaaaaaa

SEQ ID NO: 6 - protein, 367 aa, Mus musculus
MRWLLPWTLAAVAVLRVGNILATALSPTPTTMTFTPAPLEETTTRPEFCKWPCECPQSPPRCPLGVSLITDGCECCKICAQQLGDNCTEAAICDPHRGLYCDYSGDRPRYAIGVCAQVVGVGCVLDGVRYTNGESFQPNCRYNCTCIDGTVGCTPLCLSPRPPRLWCRQPRHVRVPGQCCEQWVCDDDARRPRQTALLDTRAFAASGAVEQRYENCIAYTSPWSPCSTTCGLGISTRISNVNARCWPEQESRLCNLRPCDVDIQLHIKAGKKCLAVYQPEEATNFTLAGCVSTRTYRPKYCGVCTDNRCCIPYKSKTISVDFQCPEGPGFSRQVLWINACFCNLSCRNPNDIFADLESYPDFEEIAN

SEQ ID NO: 7 - DNA, 5048 bp, Mus musculus
  source 1..5048: mol_type = genomic DNA
agaaaaagtttttttagaggaaaatgcagggctagtctgttggcctgacgtcagatgtcgctttgacaaacgcccccgggggctgaggaaggctctccgctgctctgatgggccagcccagtcctggcccagctccctggagaggcatccgcatcctctgggctgagccgtagctcctgtgacgctgacttccaggcatgaggtggctcctgccctggacgctggcagccgtggcagtcctgagggtgggcaacatcctggccacggccctctctccaacccccacaacaatgaccttcaccccagcaccactagaggaaacgactacacgccccgaattctgcaagtggccatgtgagtgcccacaatccccacctcgctgcccactgggcgtcagcctaatcacagatggctgtgaatgctgtaagatatgtgcccagcagcttggggacaactgcacagaggctgccatctgtgacccacaccggggcctctactgcgattacagtggggatcgcccgaggtacgcaataggagtgtgtgcacaggtggtcggtgtgggctgtgtcctggatggcgtacgctacaccaatggcgagtccttccaacccaactgcaggtacaactgtacctgcattgatggcacggtgggctgcacaccgctgtgcctaagccccaggcccccacgcctctggtgccgccagccccggcacgtgagagtccctggccagtgctgtgagcagtgggtgtgtgatgatgacgcaaggagaccacgccagactgcactgttggacaccagagcctttgcagcgtcaggcgccgtggagcaacggtatgagaactgcatagcctacactagtccctggagcccctgctctaccacctgtggcctaggtatctccactcggatctctaacgtcaatgcccggtgctggccagagcaggaaagtcgcctctgcaacctgcggccatgtgatgtggacatccaactacacatcaaggcagggaagaaatgcctggctgtgtaccagccagaggaggccacgaacttcactctcgcaggctgtgtcagcacacgcacctaccgacccaagtactgcggagtctgtactgacaataggtgttgcatcccctacaagtccaagaccatcagtgtggatttccagtgtccagaggggccaggtttctcccggcaggtcctatggattaatgcttgcttctgcaacctgagctgcaggaatcctaacgatatctttgctgacttggaatcttaccctgacttcgaagagattgccaattaggtgggtgtgtggctcagggtaaagttccatgctgcaaagcagccagccctttgtggtccaggacttcacaattgagccttatttcatctacttcctactcgattctgaattcccagtttctgttcctgttttgacaatcgtaatggcccaggagagtgctgctcaggctcagacaatgggttcctccttggggacattctacatcattccaaggaaaacacatctctgactgttcacaatggaagcaaagcctggcccagctagtctggctccagcctgggcaagttgtcagaagttgtgatgggattgtccaaggaaaagcatcagctgaagaaccagtatcatgaagtccttcctcagatgccaagcctagggatgctgggatcctttcagacagatggatgggattggggacacaggaataagctattattttacccttgccaaatgatactatcctgggtatttctgcctaaaacataccaaaagtgttcttgttccactgatctgtatatcacaagtcaccaaacattttccaggtgaggaccatagttgtgtcattctgttttgctattgaaaatcattttaaaaagaaggaaaaaaaaaaaagaaaagaaaagaaagtcatttcattaacttgggcactttctcctctcaccccatattctataaagggctaaacttgggttcttgttgatcaggaatgtaatttgagaagtcttacttttgcagggagatggtagccctcaattcatcccgtagggatgggacaaggccagccaatctttcaagccatagctgggcaggtcactgaatctgctgctggccaagttcttaggacaattagccaaaatctgggcctctctctccctagggttcatgggagttggtagggaccgtagagtgacttgtctgttgtctcaaaaagtaagatggaaagatgttctcatggcccttagaagactcttttgaagtctacgccagacctaacagaatatgtgcatcaaacaaacaagtggatcaccctcccatggcctgggtgacctctagcagtcacccagtgactgtggtaaggccacagtagtccctggacccaggacaaatcttttttgtttcagtgacctactttacagcctcagtgtctatgaagaaagttaactcagttttctcatctgaagacagaagtcgaccagccattcagaaatggggatctatttagtagtggtaaacgttaaagagtttgtaagctaaagaaatattttttttcctaagtgataatgaggttgttacctattttagaggtagaattttctgtcatcatccttttattgtgttctacatactacccattgttctcaatatttgccacacatttactcattgaaacttgctgataacacagaggacttcccttaatgatttctgttcttgacactgtgaaatatcaattcaaagaggctaataagttcaatcaaagtcacctatctgaagactcatagccagagaagattgccttcatatttcccctttgcttctggaccttgtaaacatgtcagggcaggaaagcatacagaagtcagcagcttgccctcttagtattggctctctagcccctttcacgtaagaggagagtgtgtctaggtactaaggatccaacctaaactggacagaaaagggcatgtgcattatcccctgtggctcctcctgccctggaatcagaactagcttacagcatgcttttatatgatgattcagtttgccccacaccatacatatgggcatatgggagaactatacacacactcataaatggatttgtaagtaggaaaggggacttgagatttcccctccttcctgcaacttcccaaaggcctggccttatgcaaggcaaacagtggtgcttaagatgtattttgggaagaaaatactatatatatatattcctatttttccttagaagccttttcttccaggatctcatcttaacagtaagaaatcctctcttaagagaggcctagtattttgagtaaacatatgttcatatgtgtacaagtacttgtatgtaaatataagttaccactgtgggttattaattaggtaaattttatgtggtatcatatttgattttcttctattggaaaaatctgccttaacagctaactctgtaagaacttccataggagccataagctagggtcttcccaggtatccatcctcttttgggaaactgagctgagcatcttcaaccaaaggagttagggtgatcattgggaataggagaaagggatggccagggtagctccatcgttatttagaaacagacctggcatacagaacgaccagaggaaccaaccttctttgaaccaagggaaaaagacttggatgtaatatatagaagctttttctaatagtcagaaacagactttaattgtatggcctggttcaaggaaagttaagaatgtccattatcgttaaaaacaaaagtccattattgatagcttatttggtgctaagccctacggtgcattttgtccggctatcacttagcagagccatgctcagtacacagtctttctctattcaataaatagaaacgctgaagctcaaaggcactgaggagctgaggctcagaagcatgtttagtccactctatcagggggggaagagatcttgacggaacctaaatgactactattggaaactcatatttgaaagctttcagaagtcccacccccaccccatccccagatgcttagaaactaaagaagcaatgaggatgagctagccttcagtgaagaggttcactactgcaccaagagtgaatgtcttaggtgtacttagtcattggacagggagacctgagtgagtttgtgaacctgcagcttactaaaccttacaatgagcatttggagagccaagactgcttctcggcgctttactgacatggcttgcttaatcttctcagtgagcccaagagtcagggcgttaccactgcccattttagggctgagaaagcaaaatctccaggagttaagtgatttgctcaagtttttaaccaaccgaggcactgcagataaactccgaagcccagtgtcatgtaacatgcccatgccatctctccggacacgcagcccattttcctgttcctaaaccaaaggctcagagtcaccagaaccaactcacaggacagtgcagaaattctaatgtcgagggtgattagagactgatcaaagaaagtaatttcaaatgatatgattgtttgtaagcaccctagttaattctggactacatatgcatagagattgtgaagaacattacagcctgtgactataacgttgacttctgtcatttctttttaaagacttgtttttttttttttactcaaaggacccacagtgacagccctgaatggttgagaagcattgattagctgtgagtcctgcatatgtatgtatgtgtgtgtgtgtgtgtgtatttgtatgtacttatctattttcaaactgtgattgtgtatttaaatattcctcctgccattttgtaagtgattacgcataaagaaacacctttgaatgtcctaataaaggagagctagcccttgggcggcctgtcacattttgccactttcctcatttttctcatgatctgtgtagcagggaatgtgtttgttcaaccatgatgagttttcattgttcaaattctttgtttacagcttttctccttaaagcaataaatcatcagcaacagtaa

SEQ ID NO: 8 - RNA, 83 nt, Homo sapiens
  source 1..83: mol_type = genomic RNA
ccttggagtaaagtagcagcacataatggtttgtggattttgaaaaggtgcaggccatattgtgctgcctcaaaaatacaagg

SEQ ID NO: 9 - RNA, 91 nt, Homo sapiens
  source 1..91: mol_type = genomic RNA
ctggggtacggggatggatggtcgaccagttggaaagtaattgtttctaatgtacttcacctggtccactagccgtccgtatccgctgcag

SEQ ID NO: 10 - RNA, 84 nt, Mus musculus
  source 1..84: mol_type = genomic RNA
cccttggagtaaagtagcagcacataatggtttgtggatgttgaaaaggtgcaggccatactgtgctgcctcaaaatacaagga

SEQ ID NO: 11 - RNA, 80 nt, Mus musculus
  source 1..80: mol_type = genomic RNA
gggtatgggacggatggtcgaccagctggaaagtaattgtttctaatgtacttcacctggtccactagccgtcggtgccc

SEQ ID NO: 12 - protein, 216 aa, Rattus rattus
EVKLLESGGGLVQPGGSMRLSCAGSGFTFTDFYMNWIRQPAGKAPEWLGFIRDKAKGYTTEYNPSVKGRFTISRDNTQNMLYLQMNTLRAEDTATYYCAREGHTAAPFDYWGQGVMVTVSSAQTTAPSVYPLAPGCGDTTSSTVTLGCLVKGYFPEPVTVTWNSGALSSDVHTFPAVLQSGLYTLTSSVTSSTWPSQTVTCNVAHPASSTKVDKKV

SEQ ID NO: 13 - protein, 216 aa, Rattus rattus; identical to SEQ ID NO: 12

SEQ ID NO: 14 - protein, 216 aa, Rattus rattus; identical to SEQ ID NO: 12

SEQ ID NO: 15 - protein, 216 aa, Rattus rattus; identical to SEQ ID NO: 12

SEQ ID NO: 16 - protein, 215 aa, Rattus norvegicus
EVKLLESGPGLVQPSQTLSLTCTVSGFPLTTNGVSWVRQPPGKGLEWIAAISSGGSPYYNSALKSRLSINRDTSKSQVFLKMNSLQTEDTAIYFCTREDGWNYFDYWGPGTMVTVSSAQTTAPSVYPLAPGCGDTTSSTVTLGCLVKGYFPEPVTVTWNSGALSSDVHTFPAVLQSGLYTLTSSVTSSTWPSQTVTCNVAHPASSTKVDKKLERR

SEQ ID NO: 17 - protein, 215 aa, Rattus norvegicus; identical to SEQ ID NO: 16

SEQ ID NO: 18 - protein, 257 aa, Rattus norvegicus
QVQLLESGPGLVRPSETLSLTCTVSGFSLTSFSVSWVRHPSGKGPEWMGRMWYDGYTAYNSALKSRLSISRDTSKNQVFLKMNSLQTDDTGTYYCTRDLYGGYPLGFWYFDFWGPGTMVTVSSGGGGSGGGGSGGGGSDIKLTQSPSLLSASVGDRVTLSCKGSQNINNYLAWYQQKLGEAPKLLIYNTNSLQTGIPSRFSGSGSGTDYTLTISSLQPEDVATYFCYQYNNGYTFGAGTKLELKAAEQKLISEEDLN

SEQ ID NO: 19 - protein, 218 aa, Rattus norvegicus
QVQLLESGPGLVRPSETLSLTCTVSGFSLTSFSVSWVRHPSGKGPEWMGRMWYDGYTAYNSALKSRLSISRDTSKNQVFLKMNSLQTDDTGTYYCTRDLYGGYPLGFWYFDFWGPGTMVTVSSVFPLAPGSAAQTNSMVTLGCLVKGYFPEPVTVTWNSGALSSGVHTFPAVLQSGLYTLTSSVTVPSSTWSSQAVTCNVAHPASSTKVDKKIVPRDC

SEQ ID NO: 20 - protein, 218 aa, Rattus norvegicus; identical to SEQ ID NO: 19

SEQ ID NO: 21 - protein, 215 aa, Rattus norvegicus
QVQLKESGPGLVQPSQTLSLTCTVSGFSLTSNSVHWVRQPPGKGLEWMGGIWGDGDTDYNSALKSRLSISRDTSKNQVFLKMNSLQTDDTAIYFCTPLIGSWYFDFWGPGTMVTASSAQTTAPSVYPLAPGCGDTTSSTVTLGCLVKGYFPEPVTVTWNSGALSSDVHTFPAVLQSGLYTLTSSVTSSTWPSQTVTCNVAHPASSTKVDQKIVPR

SEQ ID NO: 22 - protein, 215 aa, Rattus norvegicus; identical to SEQ ID NO: 21

SEQ ID NO: 23 - protein, 115 aa, Rattus norvegicus
SGPGLVQPSQTLSLTCTVSGFSLTSYGVSWVRQPPGKGLEWIGTMWNDGDTDYHSALRSRLSISRDSSKSQVLLKMNSLQTEDTAMYFCARSQLPGYNLRGWFVYWGQGTLVIVS

SEQ ID NO: 24 - protein, 252 aa, Rattus norvegicus
EVKLQESGGGLVQPGRSLKLSCAASGFTFSDSYLAWVRQAPTKGLEWVASITNSGGRFYYRDSVKGRFTISRDNAKSTLYLQMDSLRSEDTATYYCTRMDYWGQGTTVTVSSAETTAPSVYPLAPGTALKSNSMVTLGCLVKGYFPEPVTVTWNSGALSSGVHTFPAVLQSGLYTLTSSVTVPSSTWPSQTVTCNVAHPASSTKVDKKIVPRNCGLEDDDDKAGWSHPQFEKGGGSGGGSGGGSWSHPQFEK

SEQ ID NO: 25 - protein, 252 aa, Rattus norvegicus; identical to SEQ ID NO: 24

SEQ ID NO: 26 - protein, 252 aa, Rattus norvegicus; identical to SEQ ID NO: 24

SEQ ID NO: 27 - protein, 238 aa, Rattus norvegicus
MDFRLSLAFLVLLIKGVQCEVQLVESGGGLVQPGRSLKLSCAASGFTFSNYGMAWVRQTPTKGLEWIASISAGGDKTYYGDSVKGRFSISRDNAKTTHYLQMDSLRSEDTATYYCAKTSRVYFDYWGQGVMVTVSSAETTAPSVYPLAPGTALKSNSMVTLGCLVKGYFPEPVTVTWNSGALSSGVHTFPAVLQSGLYTLTSSVTVPSSTWSSQAVTCNVAHPASSTKVDKKIVPREC

SEQ ID NO: 28 - protein, 238 aa, Rattus norvegicus; identical to SEQ ID NO: 27

SEQ ID NO: 29 - protein, 242 aa, Rattus sp.
MLVLQWVLVTALFQGVHCAVQLVESGGGLVQPKESLKISCAAFGVTFSNVAMYWVRQAPGKGLEWVARIRTKPNNYATYYADSVKGRFTISRDDSKSMVYLQMDNLKTEDTAMYYCTAEVATDWGQGVMVTVSSAETTAPSVYPLAPGTALKSNSMVTLGCLVKGYFPEPVTVTWNSGALSSGVHTFPAVLQSGLYTLTSSVTVPSSTWSSQAVTCNVAHPASSTKVDKKIIVPRECNPCGC

SEQ ID NO: 30 - protein, 242 aa, Rattus sp.; identical to SEQ ID NO: 29

SEQ ID NO: 31 - protein, 219 aa, Rattus norvegicus
EVQLQESGPGLVQPSETLSLTCTVSGFSLTSYSVSWLRQPSGKGPEWMGRMWDDGGTVYNSGLKSRLSISRDTSKNQVFLKMNSLQTDDTGTYYCTRDERIRAINWFAYWGQGTLVTVSSAETTAPSVYPLAPGTALKSNSMVTLGCLVKGYFPEPVTVTWNSGALSSGVHTFPAVLQSGLYTLTSSVTVPSSTWPSQTVTCNVAHPGQQHQRWTRKLC

SEQ ID NO: 32 - protein, 214 aa, Rattus rattus
DIKMTQSPSFLSASVGDRVTLNCKASQNIDKYLNWYQQKLGESPKLLIYNTNNLQTGIPSRFSGSGSGTDFTLTISSLQPEDVATYFCLQHISRPRTFGTGTKLELKRANAAPTVSIFPPSTEQLATGGASVVCLMNKFYPRDISVKWKIDGTERNGVLNSVTDQDSADSTYSMSSTLSLTKADYQSHNLYTCQVVHKTSSSPVVAKNFNRNEC

SEQ ID NO: 33 - protein, 214 aa, Rattus rattus; identical to SEQ ID NO: 32

SEQ ID NO: 34 - protein, 214 aa, Rattus rattus; identical to SEQ ID NO: 32

SEQ ID NO: 35 - protein, 214 aa, Rattus rattus; identical to SEQ ID NO: 32

SEQ ID NO: 36 - protein, 213 aa, Rattus norvegicus
DIQMTQSPPSLSASLGDKVTITCQASQDINKYIAWYQQKPGKAPRQLIRYTSILVLGTPSRFSGSGSGRDFSFSISNVASEDIASYYCLQYGNLYTFGAGTKLEIKRADAAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNEC

SEQ ID NO: 37 - protein, 213 aa, Rattus norvegicus; identical to SEQ ID NO: 36

SEQ ID NO: 38 - protein, 66 aa, Rattus norvegicus
SVSWVRHPSGKGPEWMGRMWYDGYTAYNSALKSRLSISRDTSKNQVFLKMNSLQTDDTGTYYCTRD

SEQ ID NO: 39 - protein, 211 aa, Rattus norvegicus
DIKLTQSPSLLSASVGDRVTLSCKGSQNINNYLAWYQQKLGEAPKLLIYNTNSLQTGIPSRFSGSGSGTDYTLTISSLQPEDVATYFCYQYNNGYTFGAGTKLELKRTAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNEC

SEQ ID NO: 40 - protein, 211 aa, Rattus norvegicus; identical to SEQ ID NO: 39

SEQ ID NO: 41 - protein, 211 aa, Rattus norvegicus
DIVMTQSPSSLAVSAGERVTLNCKASQNVRNNIAWYQQKPGQSPKLLIYYASYRYTGVPDRFTGDGFGTDFTLAINSVQADDAAFYYCQRIYNSPYTFGAGTKLELIRADAAPTVSIFPPSMEQLTSGGASVVCFVNNFYPRDISVKWKIDGSEQRDGVLDSVTDQDSKDSTYSMSSTLSLTKVEYERHNLYTCEVVHKTSSSPVVKSFNR

SEQ ID NO: 42 - protein, 211 aa, Rattus norvegicus; identical to SEQ ID NO: 41

SEQ ID NO: 43 - protein, 106 aa, Rattus norvegicus
LMTQIPSLLSASVGDRVTLNCKASHNINKNLEWYQQKLGEAPKLLIYYANNLQTGISSRFSGSGSGTDYTLTISSLQPEDVATYYCYQYNSGHTFGAGTKLELKRA

SEQ ID NO: 44 - protein, 220 aa, Rattus norvegicus
RSDIVLTQTTPTLSATIGQSVSISCRSSQSLLESDGNTYLNWLLQRPGQSPQLLIYSVSNLESGVPNRFSGSGSETDFTLKISGVEAEDLGVYYCMQTTHAPTFGAGTKLELKRADAAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNEC

SEQ ID NO: 45 - protein, 220 aa, Rattus norvegicus; identical to SEQ ID NO: 44

SEQ ID NO: 46 - protein, 220 aa, Rattus norvegicus; identical to SEQ ID NO: 44

SEQ ID NO: 47 - protein, 233 aa, Rattus norvegicus
  MOD_RES 20: note = Any amino acid
MTWTLLFLAFLHHLTGSCAXFVLTQPNSVSTNLGSTVKLSCKRSTGNIGSNYVNWYQQHEGRSPTTMIYRDDKRPDGVPDRFSGSIDRSSNSALLTINNVQTEDEADYFCHSYSSGIVFGGGTKLTVLGQPKSTPTLTVFPPSTEELQGNKATLVCLISDFYPSDVEVAWKANGAPISQGVDTANPTKQGNKYIASSFLRLTAEQWRSRNSFTCQVTHEGNTVEKSLSPAECV

SEQ ID NO: 48 - protein, 233 aa, Rattus norvegicus; identical to SEQ ID NO: 47
  MOD_RES 20: note = Any amino acid

SEQ ID NO: 49 - protein, 239 aa, Rattus sp.
MMSPVQSLFLLLLWILGTNGDVVLTQAPPTLSATIGQSVSISCRSSQSLLHRNGNTYLNWLLQRPGQPPQLLIYLVSRLESGVPNRFSGSGSGTAFTLKISGLEAEDLGVYYCVQGTHAPLTFGSGTKLEIKRADAAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNEC

SEQ ID NO: 50 - protein, 239 aa, Rattus sp.; identical to SEQ ID NO: 49

SEQ ID NO: 51 - protein, 213 aa, Rattus norvegicus
DIVITQSPSLLSASVGDRVTLTCKGSQNIDNYLAWYQQKLGEAPKLLIYKTNSLQTGIPSRFSGSGSGTDYTLTISSLHSEDLATYYCYQYINGYTFGTGTKLELKRADAAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNEC

SEQ ID NO: 52 - protein, 124 aa, Ovis aries
QVQLQESGPSLMKPSQTLSLTCSVSGFSLTSYSVVWVRQAPGKALEWVGAIYSDGSASYNPALKSRLSVTRDTSKSQVSLSLSSVTTEDTAVYYCARRTVLVSMVMLICPSTTGAQGLLVTVSS

SEQ ID NO: 53 - protein, 115 aa, Ovis aries
QVQLQESGPSLVKPSQTLSLTCTVSGFSLTNYGVGVDWVRQAPGKALEWLGTIYSGGSTYYNPALKPRLSITRDTTKSQVSLSLSSVTTEDTAVYYCKRGNGDYWGPGLLVTVSS

SEQ ID NO: 54 - protein, 114 aa, Ovis aries
QVQLQESGPSLVKPSQTLSLTCTVSGFSLASYAVNWVRQAPGKALEWVGGITSGGYKYYNPALKSRLSITRDTSKSQVSLALSSVTTEDTAAYYCVRGYGGIYWGPGLLVTVSS

SEQ ID NO: 55 - protein, 120 aa, Ovis aries
QVQLQESGPSLVKPSQTLSLTCTASGFTLTSINVGWVRQAPGKVPEWLGTINSAGNTYYNPALKSRLEIVRDTSKNQFSLSLSSVTTEDTAVYYCARLYALAITMPIDYWGPGLLVTVSS

SEQ ID NO: 56 - protein, 120 aa, Ovis aries
QVQLQESGPSLVNPSRTLSLTCTVSGFSLWNHAVGWVRQAPGKEPKWLAGISSSGNTDYNPALKSRLSITRDTSKSQASLSLSSVTIEDTAVYYCVRSLVTNIGWTVDYWGPGLLVTVSS

SEQ ID NO: 57 - protein, 119 aa, Ovis aries
QVQLQESGPSLVKPSQTLSLTCTVSGFSLSTYGVGWVRQAPGRALEWVGISYNDGDTNYNPALRSRLSITRDTSKSQVSLSLSSVTAEDTAMYYCARERLYAMYDIDYWGPGLLVTVSS

SEQ ID NO: 58 - protein, 122 aa, Ovis aries
QVQLQESGPSLVKPSQTLSLTCTVSGFSLTSNAVHWVRQAPGAALEWLGSVDHDGDTDYNPALKSRLDITRDTSDKQISLSLSSVTTEDTAMYYCARDTCWGDHGVDCIDYWGPGLLVTVSS

SEQ ID NO: 59 - protein, 121 aa, Ovis aries
QVQLQESGPTLVKPSTTLSLTCTVSGFSFVNYGVTWVRQAPGKPPEWLGNIFTYGSTTYNPALKSRLSITRDTSKSQVSLSLSNMTTEETAVYHCVRNAYGGRFDLESDYWGPGLLVTVSS

SEQ ID NO: 60 - protein, 114 aa, Ovis aries
QGQLQESGPSLVKPSQTLSLTCTVSGFSLTSFAVVWVRRAPGKALEWLGDVRNNGDIDYNPALKSRLSITRDTSKSQVSLSLNSVTSEDTAVYYCARSVVAGDYWGPGLLVRLS

SEQ ID NO: 61 - protein, 122 aa, Ovis aries
QVQLQESGPSLVKPSQTLSLTCTVSGLSLTNYAVGWVRQAPGKALEWVGAIWSSGNTDYNPALKSRLGITRDTSKNQVSLSLSSVTTEDTAVYYCVRHWYDNTYGPAYIDYWGPGLLVTVSS

SEQ ID NO: 62 - protein, 120 aa, Ovis aries
QVQLQESGPSLVKPSQTLSLTCTVSGFSLTTVGVNWVRQAPGKALEWLAFIYSDGSRGYNPALKSRLSIARDTSNNSVSLSLSSVTTEDTAMYYCGRMLSIYGDAHGSHWGPGLLVTVSS

SEQ ID NO: 63 - protein, 114 aa, Ovis aries
QVQLQESGPNLVKPSQTLSLTCTVSGFPLTTCGVTWVRQAPGKALEWLGGIDSDGDVGCNQALKSRLSISRDTSKSQVSLSVSSVTIEDTAVYYCARNTYAEHWGPGLLVTVSS

SEQ ID NO: 64 - protein, 116 aa, Ovis aries
QVQLQESGPSLVKPSQTLSLTCTVSGFSLYSYDVQWVRQAPGKALEWLAWITTTGSTAYNPALKSRLSITRDTSKSQVSLLLSSVTTEDTAVYYCGRDVGSDAVMWGRRLLVTVSS

SEQ ID NO: 65 - protein, 122 aa, Ovis aries
QVQLQESGPSLVKPSQTLSLTCTVSGFSLTYNAVHWVRQAPGKVPEWLGSISSGGSTYYNAALKSRLSITRDTSKSQVSLSLSRVTTEDSAVYYCASDDGDGVVDISYIHYWGPGLLVTVSS

SEQ ID NO: 66 - protein, 118 aa, Ovis aries
QVQLQESGPSLVKPSQTLSLTCTVSGFSLTTNDVDWVRQAPGKVPEWLGEIESGGDTVYNPSLRSRLSITRDASKSQVSLSLSSVTTEDTAVYYCARWVNSYHTLDIWGRDLMVTVSS

SEQ ID NO: 67 - protein, 117 aa, Ovis aries
QVQLQESGPSLVKPSQTLSLTCTVSGFSLDSYAIGWVRQAPGRVPEWLGGISTSGSMYYNPTLKSRLSVTRDPSKSQVSLSLSSVTTDDTAMYYCAREVTAALIDYWGPGLLVTVSS

SEQ ID NO: 68 - protein, 124 aa, Ovis aries
QVQLQESGPSLVKPSQTLSLTCTVFGFSLTSNAVGWVRQAPGKVPEWLAAIGSTGSIYYAPALESRLSITRDTSKSQVSLSLSNVTSEDTAVYYCARESDWSYMFDSRSGVDGWGRRLLVTVSS

SEQ ID NO: 69 - protein, 114 aa, Ovis aries
QVQLQESGPSLVKPSQTLSLTCTVSGFSLASNAVGWVRQAPGKVPEWLCDVSTGEITYYNPALKSRLSITRDTSKSQVSLSLNSVTSDDSAVYYCARELSGKYWGPGLLVTVSS

SEQ ID NO: 70 - protein, 123 aa, Ovis aries
QVQLQESGPSLMKPSQTLSLTCSVSGFSLTSYSVVWVRQAPGKALEWVGAIYSDGSASYNPALKSRLSVTRDTSKSQVSLSLSSVTTEDTAVYYCARDRFSVYGHAYLSIDYWGPGLLVTVSS

SEQ ID NO: 71 - protein, 143 aa, Ovis aries
MNPLWTLLFVLSAPRGVLSQVRLQESGPSLVKPSQTLSLTCTVSGFSLTVNAVGWVRQAPGKVPEWLGSISTDGSTSYNPALKSRLSITRDTSKSQVSLSLSSVTTEDTAVYYCARRTFSGGGFAVGDNIDYWGPGLLVTVSS

SEQ ID NO: 72 - protein, 111 aa, Ovis aries
QAVLTQPSSVSRSLGQSVSITCFGSSSNVGYGNYVSWYQQVPGSAPKLLIYGATSRASGVPDRFSGSRFGNTAALTISSLQAEDEADYYCAFYDSSNYGVFGSGTRLTVLG

SEQ ID NO: 73 - protein, 113 aa, Ovis aries
QAVLTQPSSVSKSLGQSVSIACSGSSSNVGHGNYVSWYQQVPGSAPRILIYDATLRASGVPARFSGSRFGNAATLTITSLQAEDEADYYCASYDNSYYNGLLFGSGTRLTVLG

SEQ ID NO: 74 - protein, 110 aa, Ovis aries
QAVLTQPSSVSRSLGQSVSITCSGSSSNVGYGNFVSWYQQVPGSAPKLLIFEATSRASGVPDRFSGSRSGNTATLTIGSLQAEDEADYYCSSYDSSSFVFGSGTRLTVLG

SEQ ID NO: 75 - protein, 110 aa, Ovis aries; identical to SEQ ID NO: 74

SEQ ID NO: 76 - protein, 112 aa, Ovis aries
QAVLTQPSSVYRSMGQSVSITCSGSSSNVGYGNYVTWYQQVPGSAPKFLIYGATNRASGVPDRFSGSRPGHTATLTISSLQAEDEADYYCASYDSSTLNGVFGSGTRLTVLG

SEQ ID NO: 77 - protein, 111 aa, Ovis aries
QAVLTQPSSVSRSLGQSVSITCSGSSSNIGYGNYVSWYQQVPGSAPKILIYGATSRASGIPDRFSGSRFGNTATLTISSLQAGDESDYYCASYQTDDSEIFGSGTRLTVLG

SEQ ID NO: 78 - protein, 111 aa, Ovis aries
QAVLTQPSSVSKSLGQGVSITCSGSSSNVGYGNYVSWFQLIPGSAPKVLIYGAASRASGVPDRFSGSRSGNTATLTISSLQAEDEADYYCASYDSSSSGAFGSGTRLTVLG

SEQ ID NO: 79 - protein, 111 aa, Ovis aries
QAVLTQPSSVSKSLGQSVSITCSGSSSDVGYGNYVGWFQQVPGSAPKLLIYGATNRASGVPDRFSGSSFGNTATLTISSLQADDEADYYCASYDTSSDGVFGSGTRLTVLG

SEQ ID NO: 80 - protein, 110 aa, Ovis aries
QAVLTQPSSESRSLGQSVSITCSGSSNNVGYGNYVAWFQQIPGSAPKLLIYSGTRRAAGVPDRFSGSRSGNTATLTISSLQAEDEADYYCGSYDFSGFLFGSGTRLTVLG

SEQ ID NO: 81 - protein, 113 aa, Ovis aries
QAVLTQPSSESRSLDQSVSITCSGSSSNIGFGDHVSWFQQVPGSAPKLLIHGATNRASGVPDRFSGSRSGNTATLTISSLRTEDEADYYCASWNTGGGGGNVFGGGTRLTVLG

SEQ ID NO: 82 - protein, 111 aa, Ovis aries
QAVLTQPSSVSKSLGQSVSITCSGSSKDIGYGGSLWWLQQVPGSAPKLLIHRATNRFAGVPDRFSGSRSGSTATLTINSLQAEDEADYYCASYGSSGSGVFGSGTRLTVLG

SEQ ID NO: 83 - protein, 110 aa, Ovis aries
QAVLTQPSSESGSLGQRVSITCSGSSSNIGRGYGNWYQQVPGSAPKLLIYIATIRASGVPDRFSGSRSGNTATLTISSLQAEDEADYYCASYDGNSYGIFGSGTRLTVLG

SEQ ID NO: 84 - protein, 111 aa, Ovis aries
QAVLTQPSSVSRSLGQSVSITCSGSSSNVGIGDYVGWYQQVPGSAPKLLIYGATSRASGVPDRFSASRFGNTATLFINSLQAEDEADYYCASVDSSSGTIFGSGTRLTVLG

SEQ ID NO: 85 - protein, 112 aa, Ovis aries
QAVLTQPSTVSKSLGQSVSITCSGSSSNVGYGDYVSWYQQVPGSAPRILIYGATNRASGVPDRFTGSRFGDTATLTISSLQAEDEADYYCTTGDRSTDNGVFGSGTRLTVLG

SEQ ID NO: 86 - protein, 112 aa, Ovis aries
QAVLTQPPSVCGSLGQRVSITCSGSSSNIGGGNYVGWYQQLPGSGLKTIIYGNSSRPSGVPDRFSGSRFGNTATLTITSVQAEDEADYYCATEYSSSGNGVFGSGTRLTVLG

SEQ ID NO: 87 - protein, 108 aa, Ovis aries
QAVLTQPSSVSGSLGQRVSITCSGSNIGSNAVGWFQQVPGSGLRIIIWGSSNRPSGIPDRFSGSRSGNTATLTISSLQAEDEADYYCASYDSSSVGVFGSGTRLTVLG

SEQ ID NO: 88 - protein, 111 aa, Ovis aries
QDVLTQPSAVSRSLGQSVSTTCFGSSSNVGEDYYVNWYQQVPGSGLRTVIYYNSSRPSGVPDRFSGSKSGNTATLTISSLQAEDEADYFCGSYSGSTYGVFGSGTRLTVLG

SEQ ID NO: 89 - protein, 108 aa, Ovis aries
QAVLTQPPSVYGSLGHRVSITCFGSNIGSSTVGWFQQVPGSGLRTVIYFNDKRPSGVPDRFFGSKSGNTATLFISFIQAEDEADYFCGNWAGVGYGEFGSGTRLTVLG

SEQ ID NO: 90 - protein, 110 aa, Ovis aries
QAVLTQPFPVSRSLGQRVSITCSGSNIGSSDVGWFQQVPGSGLKTVIYFNSRRPSGVPDRFFGSKSGNTATLTISSLQAEDEADYFCGSYAGNTWNDGVFGSGTRLTVLG

SEQ ID NO: 91 - protein, 137 aa, Capra hircus
MNPLWTLLFVLSAPRGVLSQVQLQESGPSLVKPSQTLSLTCTVSGVSLTSYAVGWVRQAPGKALEWLGFIYSGGGTYYNSALKSRLSITRDTSKSQVSLSLSSVTTEDTAMYYCAGDNDDSGSVDVWGRGLLVTVSS

SEQ ID NO: 92 - protein, 146 aa, Capra hircus
MNPLWTLLFVLSAPRGVLSQVQLQESGPSLVKPSQTLSLTCTVSGFSLTSYGVNWVRQAPGKALEWLGIIRSEGSTDYNPALKSRLSITRDTSKSQVSLSLSSVTTEDTAVYYCAREYDSGNGYSSGYGPFGVDVWGRGLLVTVSS

SEQ ID NO: 93 - protein, 149 aa, Capra hircus
MNPLWTLLFVLSAPRGVLSQVQLQESGPSLVKPSQTLSLTCTVSGFSLTNYGVGWVRQAPGKALEWLGGISNSGGSTGYNPALKSRLSITRDTSKSQVSLSLSSVTTEDTAVYYCARSYGGDRGYAYAYGDLYYGVHVWGRGLLVTVSS

SEQ ID NO: 94 - protein, 130 aa, Capra hircus
MNPLWTLLFVLSAPRGVLSQVQLQESGPSLVKPSQTLSLTCTVSGVSLTSYAINWVRQAPGKALEWLGKIYSGGSTYYNSALKSRLSITGDTSKSQVSLSLSSVTTEDTAVYYCTINNVWGRGLLVTVSS

SEQ ID NO: 95 - protein, 141 aa, Capra hircus
MNPLWTLLFVLSAPRGVLSQVQLQESGPSLVKPSQTLSLTCTVSGVSLTSYAVGWVRQAPGKALEWLGGIYSSGSTYYNSALKSRLSITRDTSKSQVSLSLSSVTTEDTAVYYCARDVSYWINYNYGVDVWGRGLLVTVSS

SEQ ID NO: 96 - protein, 137 aa, Capra hircus
MNPLWTLLFVLSAPRGVLSQVQLQESGPSLVKPSQTLSLTCTVSGFSLTSNGIGWVRQAPGKAPEWVGGIDYREHTRYNPAPKSRLSITRDTSKSQVSLSLSSVTTEDTAVYYCARDLLYSVHIDAWGPGLLVTVSS

SEQ ID NO: 97 - protein, 137 aa, Capra hircus
MNPLWTLLFVLSAPRGVLSQVQLQESGPSLVKPSQTLSLTCTVSGFSLTSYDVSWVRQAPGKALEWLSSIISGGSTRYNPALKSRLSITRDTSKSQVSLSLSSVSTEDTAVYYCARGSYAIEIGDYWGPGLLVTVSS

SEQ ID NO: 98 - protein, 137 aa, Capra hircus
MNPLWTLLFVLSAPRGVLSQVQLQESGPSLVKPSQTLSLTCTVSGFSLTSYGVGWVRQAPGKAPEWVGGIDYNGYTRYNPALKSRLSFTRDTSKNQVSLSLTSVTTEDTAVYYCARDIRYLVCIDNWGPGLLVTVSS

SEQ ID NO: 99 - protein, 143 aa, Capra hircus
MNPLWTLLFVLSAPRGVLSQVQLQESGPSLVKPSQTLSLTCTVSGFSLNSYGVDWVRQTPGKALEWLGGIDGRGNTGYNPALKSRLSITRDTSKSQVSLSLSSATIEDTAVYLCTRNYYGIVSLSGGGDIDYWGPGLLVTVSS

SEQ ID NO: 100 - protein, 140 aa, Capra hircus
MNPLWTLLFVLSAPRGVLSQVRLQESGPSLVKPSQTLSLTCTVSGFSLRNYGVGWVRQAPGKAPEWVGGIDYKGYTRYNPALKSRLSFTRDASKSQVSLSLTSVTTEDTAVYYCVRYYNDHDNAYGMNDWGPGLLVTVSS

SEQ ID NO: 101 - protein, 136 aa, Capra hircus
MNPLWTLLFVLSAPRGVLSQVQLQESGPSLVKPSQTLSLTCTVSGFSLTSHTVNWVRQAPGKALEWLGARGISGSTYYNSALKSRLSFTRDTSKSQVSLSLSSVTTEDTAVYYCAGDISTIVFDYWGPGLLVTVSS

SEQ ID NO: 102 - protein, 130 aa, Capra hircus
MNPLWTLLFVLSAPRGVLSQVQLQESGPSLVKPSQTLSLTCTVSGASLTTYHVEWVRQAPGKALEWLGIIWSDGGTNYNSALKSRLSIIRDTSKSQVSLSLSSVTTEDTAVYYCGINLIWGRGLLVTVSS

SEQ ID NO: 103 - protein, 140 aa, Capra hircus
MNPLWTLLFVLSAPRGVLSQVQLQESGPSLVKPSQTLSLTCTVSGVSLTNYAVGWVRQAPGGALEWLGIIWTSAGTHYNSALKSRLSITGDTSKRQVSLSLSSVTTEDTAVYYCVRAGSPNSYAGGLDVWGRGLLVTVSS

SEQ ID NO: 104 - protein, 142 aa, Capra hircus
MNPLWTLLFVLSAPRGVLSQVQLQESGPSLVKPSQTLSLTCTVSGFSLTTYAVGWVRQAPGKALEWLGGIDRDGNRGYNPALKSRLSITRGTSKSQVSLSLSSVTSEDTSVYYCTKSSSGYGHGHGEGYEVWGRGLLVTVSS

SEQ ID NO: 105 - protein, 136 aa, Capra hircus
MNPLWTLLFVLSAPRGALSQVQLQESGPSLVKPSQTLSLTCTVSGFSLISYRVGWVRQAPGKALEWLGGIDRDGSTGYNSALKSRLSITRDTSKSQLSLSLSSVTTEDTAVYYCARMIYATDVTVWGRGLLVTVSS

SEQ ID NO: 106 - protein, 145 aa, Capra hircus
MNPLWTLLFVLSAPRGVLSQVQLQESGPSLVKPSQTLSLTCTVSGFSLTSYGVGWVRQAPGKALEWLGGISSGGSTGYNPALKSRLSITRDTSKSQVSLSLSSVTTEDTAVYYCARGGSGYVYDYGYGWSGIDAWGRGLLVTVSS

SEQ ID NO: 107 - protein, 131 aa, Capra hircus
MNPLWTLLFVLSAPRGVLSQVQLQESGPSLVKPSQTLSLTCTVSGFSLTSYGVVWVRQAPGKALEWLGGITSGGATYYNSALKSRLSITRDTSKSQVSLSLSSVTTEDTAVYYCARYIDVWGRGLLVTVSS

SEQ ID NO: 108 - protein, 145 aa, Capra hircus
MNPLWTLLFVLSAPRGVLSQVQLQESGPSLVKPSQTLSLTCTVSGFSLTSYGVGWVRQAPGKALEWLGGINSGGSTYYNPALKSRLSITGDTSKSQVSLSLSSVTTEDTAVYYCAIYYSTYGYDYGYDFDGVYVWGRGLLVTVSS

SEQ ID NO: 109 - protein, 143 aa, Capra hircus
MNPLWTLLFVLSAPRGVLSQVQLQESGPSLVKPSQTLSLTCTVSGVSLTSYAVGWVRQAPGKALEWLGDIAGGGSTYYNSALKSRLSITRDTSKSQVSLSLKSVTTEDTAVYYCGTYGYGYGYVYGYYSVDVWGRGLLVTVSS

SEQ ID NO: 110 - protein, 132 aa, Capra hircus
MNPLWTLLFVLSAPRGVLSQVQLQESGPSLVKPSQTLSLTCTVSGFSLTSYGVGWVRQAPGKALEWLGGISSGGSTYYNSALKSRLSITRDTSKSQVSLSLSSVTTEDTAVYYCAGSGYGVWGPGLLVTVSS

SEQ ID NO: 111 - skipped

SEQ ID NO: 112 - protein, 117 aa, unidentified
  REGION 1..117: note = Description of Unknown: Leporidae sequence
ESVKESEGGLFKPTDTLTLTCTVSGFSLSGYDMSWVRQAPGKGLEWIGVIYASGSTYYATWAKSRSTITRTSBTVBLMDSLTAQDTATYFCARGHTGLSYLKSSVDVWGPGTLVTVS

SEQ ID NO: 113 - protein, 114 aa, unidentified
  REGION 1..114: note = Description of Unknown: Leporidae sequence
ESESGGRLVTPTPGLTLTCTVSGFSLSSYDMGWVRQAPGKGLEWIGIIYASGSTYYASWAKGRFTISKTSTTVDLKTSLPTEDTATYFCARQGTGLVHLAFVDVWGPGTLVTVS

SEQ ID NO: 114-121 - skipped (8 entries)

SEQ ID NO: 122 - protein, 110 aa, unidentified
  REGION 1..110: note = Description of Unknown: Leporidae sequence
IVMTQTPSSKSVPVGDTVTINCQASQSVYSNNRLAWFQQKPGQPPKLLIYKASTLASGVPSRFKGSGSGTQFTLTISDVQCADAATYYCRVASTNNIVFGGGTEVVVKGD

SEQ ID NO: 123 - protein, 115 aa, unidentified
  REGION 1..115: note = Description of Unknown: Leporidae sequence
IVMTQTPSSKSVPVGDTVTINCQAAQSVYSNNRLSWFQQKPGQPPKGLIYYASTLASGVQQDPSRFKGSGSGTQFTLTISDVQCBBAATVYYCQGYKSSDTRAFGGGTEVVVKGD

SEQ ID NO: 124 - protein, 110 aa, unidentified
  REGION 1..110: note = Description of Unknown: Leporidae sequence
ADIVMTQTPASVEAAVGGTVTIKCQASQSIGNFLSWYQQKPGQPPKLLIYKASTLASGVPSRFKGSGSGAEFTLPISDVQCADSSTYYCQQGNLGNIVFGGGTEVVVKGD

SEQ ID NO: 125 - protein, 109 aa, unidentified
  REGION 1..109: note = Description of Unknown: Leporidae sequence
DVVMTQTPASVSEPVGGTVTIKCQASQSIYSGLAWYQQKPGQPPKLLIYKASTLASGVSSRFKGSGSGTEFTLTISDLECADAATYFCQGSTYGGGYFGGGTEVVVKGD

SEQ ID NO: 126 - protein, 109 aa, unidentified
  REGION 1..109: note = Description of Unknown: Leporidae sequence
DVVMTQTPASVSEPVGGTVTIKCQASQSIYSNLAWYQQKPGQPPKLLIYKASTLESGVPSRFKGSGSGTDFTLTISDLECADAATYFCQGSBYTGTVFGGGTEVVVKGD

SEQ ID NO: 127 - protein, 109 aa, unidentified
  REGION 1..109: note = Description of Unknown: Leporidae sequence
AYDMTQTPSSVSAAVGGTVTINCQASEDISANLAWYQQKPGQPPKLLIYAASDLASGVPSRFKGSGSGTEYTLTISGVQCADAATYYCQSADYSGSAVTFGGGTEVVVK

SEQ ID NO: 128 - protein, 110 aa, unidentified
  REGION 1..110: note = Description of Unknown: Leporidae sequence
ADIVMTQTPASVSAAVGGTVTINCQASQNIDSWLAWYQQKPGQPPKVLIYRTSTLASGVPSRFKGSRSGTEFTLTISDLECADAATYYCQSYYSISSAFGGGTEVVVKGD

SEQ ID NO: 129 - skipped

SEQ ID NO: 130 - protein, 111 aa, unidentified
  REGION 1..111: note = Description of Unknown: Leporidae sequence
VEVLTQTPSPVSAAVGGTVTISCQSTKSIYBBBYLAWYQZKPGQPPKALIYTASSLASGVPSRFTGSGSGTZFTLTLSDVZCDDAATYYCGGADYTGTSFGGGTEVVVKGB

SEQ ID NO: 131 - protein, 109 aa, unidentified
  REGION 1..109: note = Description of Unknown: Leporidae sequence
AFELTQTPSSVEAAVGGTVTIKCQSSQSIGTYLAWYZZKPGQPPKLLIYRASTLASGVSSRFKGSGSGTEFTLTISGVECADAATYYCQGTYYFSASFGGGTEVVVKGD

SEQ ID NO: 132 - protein, 98 aa, unidentified
  REGION 1..98: note = Description of Unknown: Leporidae sequence
ADIVMTQTPASVSEPVGGTVTIKQASQSIFBBLAWYQQKPGZPPKGLLYTBYLAGVSSRFSGGGSGTBFTLTISDLEABAATYYETGVSZBBKGFGGG

SEQ ID NO: 133 - protein, 111 aa, unidentified
  REGION 1..111: note = Description of Unknown: Leporidae sequence
DVVMTQTPSSKSAAVGDTVTIKCQASQSVYSNNYLSWYQQKPGQPPKLLIYDASKLASGVPSRFKGSGSGTQFTLTISGVQCDDAATYYCQGTYYSSGWYTFGGGTKVVVE

SEQ ID NO: 134 - protein, 112 aa, unidentified
  REGION 1..112: note = Description of Unknown: Leporidae sequence
AQVLTQTASPVSAAVGGTVTINCQSSQSVYNNNKLSWFQQKPGQPPKQLIYSASSLASGVPSRFSGSGSGTQFTLTISCVQCNDAATYSCQGAYSGATYGNTFGGGTKVVVE

SEQ ID NO: 135 - protein, 110 aa, unidentified
  REGION 1..110: note = Description of Unknown: Leporidae sequence
AAVLTQTPSPVSVAVGGTVTISCQSSQSVYNNNDLAWYQQKPGQPPKLLIYRASKLASGVPSRFSGSGSGTQFTLTISGVQCDDAATYYCLGGYDDDADTFGGGTKVVVE

SEQ ID NO: 136 - protein, 110 aa, unidentified
  REGION 1..110: note = Description of Unknown: Leporidae sequence
DVVMTQTPSSKSAAVGDTVTIKCQASQSISSYLSWYQQKPGQPPKLLIYYASDLASGVPSRFKGSGSGTEFTLTISGVQCDDAATYYCQGGYYTSSADNTFGGGTKVVVE

SEQ ID NO: 137 - protein, 112 aa, unidentified
  REGION 1..112: note = Description of Unknown: Leporidae sequence
AQVLTQTPSSVSAAVGGTVTINCQSSQSVYSNNYLSWYQQKPGQPPKLLIYDASKLASGVPSRFKGSGSGTQFTLTISGVQCDDAATYYCQGTYYSSGWYNTFGGGTKVVVE

SEQ ID NO: 138 - protein, 113 aa, unidentified
  REGION 1..113: note = Description of Unknown: Leporidae sequence
AAVLTQTPSPVSAAVGGTVTISCQSSQSVYNNNNLAWYQQKPGQPPKLLIYSASSLASGVPSRFKGSGSGTQFTLTISDLECDDAATYYCAGGYYCSSADCNTFGGGTKVVVE

SEQ ID NO: 139 - protein, 109 aa, unidentified
  REGION 1..109: note = Description of Unknown: Leporidae sequence
AQVLTQTPSSVSEPVGGTVTINCQASENIYSSLAWYQQKPGQPPKLLIYDASDLASGVPSRFSGSGSGTEFTLTISGVQCDDAATYYCQSYYYSSVTNTFGGGTKVVVE

SEQ ID NO: 140 - protein, 111 aa, unidentified
  REGION 1..111: note = Description of Unknown: Leporidae sequence
AQVLTQTPSSVSAAVGGTVTINCQSSQSVYSNNYLSWYQQKPGQPPKLLIYDASKLASGVPSRFKGSGSGTQFTLTISGVQCDDAATYYCLGGYDDDADNTFGGGTKVVVE

SEQ ID NO: 141 - protein, 111 aa, unidentified
  REGION 1..111: note = Description of Unknown: Leporidae sequence
AAVLTQTPSPVSAAVGGTVTISCQSSQSVYNNNDLAWYQQKPGQPPKLLIYRASKLASGVPSRFSGSGSGTQFTLTISGVQCDDAATYYCLGGYDDDADNTFGGGTKVVVE

SEQ ID NO: 142 - RNA, 22 nt, Mus musculus
  source 1..22: mol_type = genomic RNA
tagcagcacataatggtttgtg

SEQ ID NO: 143 - RNA, 23 nt, Mus musculus
  source 1..23: mol_type = genomic RNA
aatggcccaggagagtgctgctc

SEQ ID NO: 144 - RNA, 24 nt, Mus musculus
  source 1..24: mol_type = genomic RNA
ggcaggtcactgaatctgctgctg

SEQ ID NO: 145 - DNA, 82 bp, Mus musculus
  source 1..82: mol_type = genomic DNA
ccttggagtaaagtagcagcacataatggtttgtggatgttgaaaaggtgcaggccatactgtgctgcctcaaaatacaagg

SEQ ID NO: 146 - DNA, 83 bp, Homo sapiens
  source 1..83: mol_type = genomic DNA
ccttggagtaaagtagcagcacataatggtttgtggattttgaaaaggtgcaggccatattgtgctgcctcaaaaatacaagg

SEQ ID NO: 147 - DNA, 73 bp, Mus musculus
  source 1..73: mol_type = genomic DNA
gggtatgggacggatggtcgaccagctggaaagtaattgtttctaatgtacttcacctggtccactagccgtc

SEQ ID NO: 148 - DNA, 74 bp, Homo sapiens
  source 1..74: mol_type = genomic DNA
gggtacggggatggatggtcgaccagttggaaagtaattgtttctaatgtacttcacctggtccactagccgtc